SEQUENCE LISTING (40 sequences)

SEQ ID NO: 1 - DNA, 20 bp, Artificial Sequence
  beta actin PCR primer sequence 1
cgaccactttgtcaagctca

SEQ ID NO: 2 - DNA, 23 bp, Artificial Sequence
  beta actin PCR primer sequence 2
ggttgagcacagggtactttatt

SEQ ID NO: 3 - DNA, 23 bp, Artificial Sequence
  RQCD1 PCR primer sequence 1
agaccctaaagatcgtccttctg

SEQ ID NO: 4 - DNA, 23 bp, Artificial Sequence
  RQCD1 PCR primer sequence 2
gtgttttaagtcagcatgagcag

SEQ ID NO: 5 - DNA, 21 bp, Artificial Sequence
  beta actin PCR primer sequence 3
atggaaatcccatcaccatct

SEQ ID NO: 6 - DNA, 20 bp, Artificial Sequence
  RQCD1 PCR primer sequence 3
gccttcatcatccaaacatt

SEQ ID NO: 7 - DNA, 20 bp, Artificial Sequence
  RQCD1 PCR primer sequence 4
ggcaaatatgtctgccttgt

SEQ ID NO: 8 - DNA, 21 bp, Artificial Sequence
  si-#1 target sequence
aagatctatcagtggatcaat

SEQ ID NO: 9 - DNA, 21 bp, Artificial Sequence
  si-#2 target sequence
aagatcttgttagatgacact

SEQ ID NO: 10 - DNA, 900 bp, Homo sapiens
  CDS (1)..(900)
atgcacagcctggcgacggctgcgcctgtgcctactacactggcacaagtggatagagaaaagatctatcagtggatcaatgagctgtccagtcctgagactagggaaaatgctttgctggagctaagtaagaagcgagaatctgttcctgaccttgcacccatgctgtggcattcatttggtactattgcagcacttttacaggaaattgtaaatatttatccatctatcaacccacccaccttgacagcacaccagtctaacagagtttgcaatgctctggcattactgcaatgtgtagcatcacatccagaaaccaggtcagcgtttctcgcagcacacatcccactttttttgtacccctttttgcacactgtcagcaaaacacgtccctttgagtatctccggctcaccagccttggagttattggggccctggtgaaaacagatgaacaagaagtaatcaactttttattaacaacagaaattatccctttatgtttgcgaattatggaatctggaagtgaactttctaaaacagttgccacattcatcctccagaagatcttgttagatgacactggtttggcttatatatgtcagacgtatgagcgtttctcccatgttgccatgatcttgggtaagatggtcctgcagctatccaaagagccttctgcccgtctgctgaagcatgtagtgagatgttaccttcgactttcagataaccccagggcacgtgaagcactcagacagtgcctccctgaccagctgaaagacacaaccttcgcccaggtgctaaaagatgacaccaccacgaaacgctggcttgcacaactggtgaagaacctgcaagagggccaggtcaccgatccccggggtatccccctgccccctcagtga

SEQ ID NO: 11 - protein, 299 aa, Homo sapiens
MHSLATAAPVPTTLAQVDREKIYQWINELSSPETRENALLELSKKRESVPDLAPMLWHSFGTIAALLQEIVNIYPSINPPTLTAHQSNRVCNALALLQCVASHPETRSAFLAAHIPLFLYPFLHTVSKTRPFEYLRLTSLGVIGALVKTDEQEVINFLLTTEIIPLCLRIMESGSELSKTVATFILQKILLDDTGLAYICQTYERFSHVAMILGKMVLQLSKEPSARLLKHVVRCYLRLSDNPRAREALRQCLPDQLKDTTFAQVLKDDTTTKRWLAQLVKNLQEGQVTDPRGIPLPPQ

SEQ ID NO: 12 - DNA, 20 bp, Artificial Sequence
  ACTB PCR primer sequence
ggaacggtgaaggtgacagc

SEQ ID NO: 13 - DNA, 20 bp, Artificial Sequence
  ACTB PCR primer sequence
acctcccctgtgtggacttg

SEQ ID NO: 14 - DNA, 23 bp, Artificial Sequence
  RQCD1 primer sequence
ggacttgttagttggcttctgtc

SEQ ID NO: 15 - DNA, 22 bp, Artificial Sequence
  RQCD1 primer sequence
gatcacttctcttcaggcttgc

SEQ ID NO: 16 - DNA, 22 bp, Artificial Sequence
  RQCD1 primer sequence
ctggcacaagtggatagagaaa

SEQ ID NO: 17 - DNA, 22 bp, Artificial Sequence
  RQCD1 primer sequence
cagaaggctctttggatagctg

SEQ ID NO: 18 - DNA, 22 bp, Artificial Sequence
  GIGYF1 primer sequence
cagcagagacactcaactttgg

SEQ ID NO: 19 - DNA, 23 bp, Artificial Sequence
  GIGYF1 primer sequence
cttcttcgatgcttctttggtaa

SEQ ID NO: 20 - DNA, 21 bp, Artificial Sequence
  GIGYF2 primer sequence
cggcagagaagaaatgttagc

SEQ ID NO: 21 - DNA, 22 bp, Artificial Sequence
  GIGYF2 primer sequence
gctttctccctactgatgttgg

SEQ ID NO: 22 - DNA, 26 bp, Artificial Sequence
  primer sequence for RACE
gcggcaaccctgtaattcccatagac

SEQ ID NO: 23 - DNA, 26 bp, Artificial Sequence
  primer sequence for RACE
ggaggtgcttgggattaaggtgacag

SEQ ID NO: 24 - DNA, 27 bp, Artificial Sequence
  RQCD1 primer sequence
ggaattcaatgcacagcctggcgacgg

SEQ ID NO: 25 - DNA, 28 bp, Artificial Sequence
  RQCD1 primer sequence
ggactcgagctgagggggcagggggata

SEQ ID NO: 26 - DNA, 39 bp, Artificial Sequence
  GIGYF1 primer sequence
gttaagtagcggccgctcatggcagcagagacactcaac

SEQ ID NO: 27 - DNA, 27 bp, Artificial Sequence
  GIGYF1 primer sequence
ccgctcgaggtagtcatccacgctctc

SEQ ID NO: 28 - DNA, 38 bp, Artificial Sequence
  GIGYF2 primer sequence
gttaagtagcggccgctcatggcagcggaaacgcagac

SEQ ID NO: 29 - DNA, 27 bp, Artificial Sequence
  GIGYF2 primer sequence
ccgctcgaggtagtcatccaacgtctc

SEQ ID NO: 30 - DNA, 19 bp, Artificial Sequence
  siRNA target sequence for RQCD1
gatctatcagtggatcaat

SEQ ID NO: 31 - DNA, 19 bp, Artificial Sequence
  siRNA target sequence for RQCD1
gatcttgttagatgacact

SEQ ID NO: 32 - DNA, 19 bp, Artificial Sequence
  siRNA target sequence for GIGYF1
ccttccgaagggctagagg

SEQ ID NO: 33 - DNA, 19 bp, Artificial Sequence
  siRNA target sequence for GIGYF2
caagataccttcagacctt

SEQ ID NO: 34 - DNA, 19 bp, Artificial Sequence
  siRNA target sequence for EGFP
gcagcacgacttcttcaag

SEQ ID NO: 35 - DNA, 6329 bp, Homo sapiens
  CDS (1010)..(4117)
gattgggggcggtggtctgggtgtgattcctgtgggggaaaccagcccgaggtgccttggctcaggcttgcctgcctggagtccttgtcgtgtacccgtctgtggccctagagtcacctcctgctccttttcactgggtttctccccagaccaagcccctggatttgagcttctgagcagccaactgatttttttcatgagggaaagaattttgctcctagatttatttctaatccttttctccgtaactgaccttttctctgtaagctatttgtggctctagtgggtttttgtttgcttgcttgtttttgttttatctttgccctgaacgtccatcctggcctcggctgcctccctccccgagttcttcactacacctcctgtctcagcctcccagagagggtggcgagggtgctgatgcccagagacaccatccggcgaatggacctctcctgtcatgcggggcagggcagggctgccggcagagccatgtcctgaaccctccctcctcccccctgctctccttaagggtctttcccagatgtgaaggtccctcccaggctctactgaggtcagaccaattcccagtggcttgggtaccctcgttcactccagcaccagcccctgccctccttcttcccgggccaggctgccaggtgagccccgggccaagggtggttgccctgctgaggccgaaaggactcgagcttcccatctcctcgcctcccccaggtctttcaccgtctggactcatctctgggtaactcatggtgtgagtggccacgtggctggctcaggtgagtccttttagaccccaggtgacccaccagctagtccggggcccattgttgtgcccactaggatgtcccccatggtgtcctccctgcgctttaaagttctccttcgtgtgctgttcttcatggcccctgttcccccacaggtgtttggacagtgaacgccaggtcccctcctccccacccccggcctctcaaacacgcccagccccacgatggcagcagagacactcaactttgggcctgagtggctcagggccctgtccgggggcggcagcgtggcctccccacccccgtcccctgccatgcccaaatacaagctggctgactaccgttatgggcgagaggaaatgctggctctctacgtcaaggagaacaaggtcccggaagagctgcaggacaaggagttcgccgcggtgctgcaggacgagccactgcagcccctggctctggagccgctgactgaggaggaacagagaaacttctccctgtcagtgaacagcgtggctgtgctgaggctgatggggaaaggggctggcccccccctggctggcacctcccgaggcaggggcagcacgcggagccgaggccgcggccgtggtgacagctgcttttaccaaagaagcatcgaagaaggcgatggggcctttggacgaagcccccgggaaatccagcgcagccagagctgggatgacagaggcgagaggcggtttgagaagtcagcaaggcgggatggagcacgatgtggctttgaggagggaggggctggcccaaggaaggagcacgcccgctcagacagcgagaactggcgctccctacgggaggaacaggaggaggaggaggagggcagctggaggctcggagcagggccccggcgagacggcgaccgctggcgctccgccagccctgatggtggtccccgctctgctggctggcgggaacatggggaacggcggcgcaagtttgaatttgatttgcgaggggatcgaggagggtgtggtgaagaggaggggcggggagggggaggcagctctcacctgcggcggtgccgagcgcctgaaggctttgaggaggacaaggatgggctcccagagtggtgcctggacgatgaggatgaagaaatgggcacctttgatgcctctggggccttcttgcctctcaagaagggccccaaggagcccattcctgaggagcaggagctggacttccaagggttggaggaggaggaggaaccttccgaagggctagaggaggaagggcctgaggcaggtgggaaagagctgaccccactgcctcctcaggaggagaagtccagctccccatccccactgcccaccctgggcccactctgggggacaaacggggatggggacgaaactgcagagaaagagcccccagcggccgaagatgatattcgggggatccagctgagtcccggggtgggctcctctgctggcccacccggagatctggaggatgatgaaggcttgaagcacctgcagcaggaggcggagaagctggtggcctccctgcaggacagctccttggaggaggagcagttcacggctgccatgcagacccagggcctgcgccactctgcagccgccactgccctcccgctcagccatggggctgcccggaagtggttctacaaggacccacagggcgagatccaaggccccttcacgacacaggagatggcagagtggttccaggccggctacttttccatgtcactgctggtgaagcggggctgcgatgagggcttccagccgctgggcgaggtgatcaagatgtggggccgcgtgccctttgccccagggccctcacctcccccactgctgggaaacatggaccaggagcggctgaagaagcaacaggagctggccgcggcggccttgtaccagcagctgcagcaccagcagtttctccagctggtcagcagccgccagctcccacagtgcgcgctccgagaaaaggcagctctgggggacctgacaccgccaccaccgccgccgccacagcagcagcagcagcagctcacggcattcctgcagcagctccaggcgctcaaaccccccagaggcggggaccagaacctgctcccgacgatgagccggtccttgtcggtgccagattcgggccgcctctgggacgtacatacctcagcctcatcacagtcaggtggtgaggccagtctttgggacataccaattaactcttcgactcagggtccaattctagaacaactccagctgcaacataaattccaggagcgcagagaagtggagctcagggcgaagcgggaggaagaggaacgcaagcgtcgagaggagaagcgccgccagcagcagcaggaggagcagaagcggcggcaggaggaggaagagctgtttcggcgcaagcacgtgcggcagcaggagctattgctgaagttgctacagcagcagcaggcggtccctgtgccccccgcacccagctccccgcccccactctgggctggcctggccaagcaggggctgtccatgaagacgctcctggagttgcagctggagggcgagcggcagctgcacaaacagcccccacctcgggagccagctcgggcccaggcccccaaccaccgagtgcagcttgggggcctgggcactgcccccctgaaccagtgggtgtctgaggctgggccactgtggggcgggccagacaagagtgggggcggcagcagcggcctggggctctgggaggacacccccaagagcggcgggagcctggtccgtggcctcggcctgaagaacagccggagcagcccatctctcagtgactcatacagccacctatcgggtcggcccattcgcaaaaagacggaggaagaagagaagctgctgaagctgctgcagggcattcccaggccccaggacggcttcacccagtggtgcgagcagatgctgcacacgctgagcgccacgggcagcctggacgtgcccatggctgtagcgatcctcaaggaggtggaatccccctatgatgtccacgattatatccgttcctgcctgggggacacgctggaagccaaagaatttgccaaacaattcctggagcggagggccaagcagaaagccagccagcagcggcagcagcagcaggaggcatggctgagcagcgcctcgctgcagacggccttccaggccaaccacagcaccaaactcggccccggggagggcagcaaggccaagaggcgggcactgatgctgcactcagaccccagcatcctggggtactccctgcacggatcttctggtgagatcgagagcgtggatgactactgaccagcccggacccccagcccctgggctgtaggccagggcagccacagcggcgtggaccgagggtcccagcctgcaggctccccgcagagagcacaggaagaggcaggggcggggtccccagcacttgttacaaacacacgatgcaccttaactcacccaccacgaggcactttacagactgggggagggggtttttctttttatttttttttttaattttaaacgactgaagaaaacattaggagaggcaaaaatattgttaaaaactagactctaaacaccccttcctgctgtgaggatagtgggtgtgacaatggaaggtccacagaggtttttgttttttggttttttttttttttttaagaaaaaaagatgaaaaatgaaaaaaaaaatggttaggaggctgaaagaaaaaacacactgttattttggggcagtggggacacaggccccgtggacctgtcctgcctggcccccaaggccatacttaccccccagaaggcgggccatggggtaactggaagctgggggccagcagtttgcacaggaggcctgtctgagccccacccgccagacctgttgtgagcagctcctgtcactgaggctggctgaggtgtccggggtggggccaaagtagccccttggcttcgctgctttgggggacagttgcacaaattggacgagtggccccagctctctggctgccatcttgtgctggccgagtagacgggaggggccaagccgtgccaacctctctggctggcagggtggggcagcaggactctggttctggtgaggggcgtctcccactgctgccattttgggggacaccctgggtttgaacctgaaagccccagctctctgccttgccacgtgaatgtattctttgggccacaagcccccgcctcacccctgcctgagctgcctcacccctgtgagcggcgggggtggatgattgctccagaggctgcagagagaaggctgagctgtttctccagtgaagggggcaggaggaggggcctcaaaggcaaggagtgggtggctttgggcttagggttgcagtagaggggctgccgcccggggccccaacctgtagccagctcgtaagatgtggaccacccagctctgcacctgacccttccgctgaccaaatgggagaggagcaggtggccttccgggtctgatatgatgcgctttttaccgttgggtaaggttggggtgaagagaagtgtgcggctcctgggtcagaggaggctgccccttctattgctcacccacttcttattcccggtcccctacttgggttcgtctccgcccattttgggttttgtaacagttttgtcttttgggtttctcatccagctcctcccattgacctcattgctcagagtgcagtattagggcaaggcttcgccactgcctccctccatgaatgtatttctccctcctgccctggggacatggggagtggcccgtttctttccccatctagtcccagaaagatggtgtttggttttctgttgttggatttttttttttttttttttttttttgcaccaaagtggcaactaggtcagtgttgggggatcaagctggcctcggggtggggggcccccacctgcctctccctggttcccacagtgttagcgtccctgaaaagacaatattctctctaaagcaataaggggtgacgggccggggggagtgtttgctgctgctggcccccagctccccttccctcttgccaggtgtgggggagactcctgttgtgactgaatgtaacccccccacccctgccgcagccaatgcaggggaagggggacactcttcctgtctcttctccccagctaaagagactttggacttagggggcccatgagcctggagaggccttaaccctgtgaggaagtatagggggagccctctcccacccccatccccttctgagagtggtcaatgtttacaagcccctgagcccccctgcccagggactcagaccctgttgctgtccttccccggccccggtcttcctgggccctcgctgctcccctgcccttcctggggttggggtgggtgcaggggtcaccgtgttccctgtctgccttgtacccacagtctccccgccccctctccaccctgtgtgacttccctctcttttacctgctcctgtaaatactcccttctcccaataaaacttggtgtgtgttctccc

SEQ ID NO: 36 - protein, 1035 aa, Homo sapiens
MAAETLNFGPEWLRALSGGGSVASPPPSPAMPKYKLADYRYGREEMLALYVKENKVPEELQDKEFAAVLQDEPLQPLALEPLTEEEQRNFSLSVNSVAVLRLMGKGAGPPLAGTSRGRGSTRSRGRGRGDSCFYQRSIEEGDGAFGRSPREIQRSQSWDDRGERRFEKSARRDGARCGFEEGGAGPRKEHARSDSENWRSLREEQEEEEEGSWRLGAGPRRDGDRWRSASPDGGPRSAGWREHGERRRKFEFDLRGDRGGCGEEEGRGGGGSSHLRRCRAPEGFEEDKDGLPEWCLDDEDEEMGTFDASGAFLPLKKGPKEPIPEEQELDFQGLEEEEEPSEGLEEEGPEAGGKELTPLPPQEEKSSSPSPLPTLGPLWGTNGDGDETAEKEPPAAEDDIRGIQLSPGVGSSAGPPGDLEDDEGLKHLQQEAEKLVASLQDSSLEEEQFTAAMQTQGLRHSAAATALPLSHGAARKWFYKDPQGEIQGPFTTQEMAEWFQAGYFSMSLLVKRGCDEGFQPLGEVIKMWGRVPFAPGPSPPPLLGNMDQERLKKQQELAAAALYQQLQHQQFLQLVSSRQLPQCALREKAALGDLTPPPPPPPQQQQQQLTAFLQQLQALKPPRGGDQNLLPTMSRSLSVPDSGRLWDVHTSASSQSGGEASLWDIPINSSTQGPILEQLQLQHKFQERREVELRAKREEEERKRREEKRRQQQQEEQKRRQEEEELFRRKHVRQQELLLKLLQQQQAVPVPPAPSSPPPLWAGLAKQGLSMKTLLELQLEGERQLHKQPPPREPARAQAPNHRVQLGGLGTAPLNQWVSEAGPLWGGPDKSGGGSSGLGLWEDTPKSGGSLVRGLGLKNSRSSPSLSDSYSHLSGRPIRKKTEEEEKLLKLLQGIPRPQDGFTQWCEQMLHTLSATGSLDVPMAVAILKEVESPYDVHDYIRSCLGDTLEAKEFAKQFLERRAKQKASQQRQQQQEAWLSSASLQTAFQANHSTKLGPGEGSKAKRRALMLHSDPSILGYSLHGSSGEIESVDDY

SEQ ID NO: 37 - DNA, 7959 bp, Homo sapiens
  CDS (338)..(4237)
gtgacgtgcgtggcgacgtgtcggccatcttgtgttgttgaggctgaggactgactggggttctgagactccctgtcccggaccgcagattatagtgggaccagtctcattaggttgaatctacagcctatgttggtgttaacccaggtctcttagagcgttaaaaggatctgaacaaagtctgctcaaatctcctgctgtgaaccagcagaatttttgaacagagaccacgtctccacctcctgggctccaacgattctcccatcttggcctcccaaagcgctggatttacaggtttcttcacatataaaaatctattgtaaaaatacggaaaagaatggcagcggaaacgcagacactgaactttgggcctgaatggctccgagctctgtccagtggtgggagtattacatcccctcctctttctccagcattgccgaagtataaattagcagattatcgttacggcagagaagaaatgttagcacttttccttaaagacaacaagataccttcagaccttctggataaagaatttctgcctatcctccaggaggaaccccttccaccattggctctggtaccctttacagaagaagaacagagaaacttttccatgtctgtaaatagtgctgctgtcctgcgattgacaggacgaggaggaggaggaacagtggtgggggctcctagaggtcgaagttcttcaagagggcgaggcagaggcagaggtgaatgtggtttctaccaaagaagttttgatgaagtagagggtgtttttggtcgaggaggtggcagagaaatgcatagatcgcagagctgggaggaaaggggtgacagacgttttgaaaaaccaggacgaaaagatgtagggagaccaaattttgaggaaggtggaccaacatcagtagggagaaagcatgaatttatacgctcagaaagtgaaaattggcgcatctttagagaggaacaaaatggagaagatgaagatggaggttggcgactagctggatcaaggagggatggagagaggtggcgacctcacagtcctgatggccctcgttctgcaggctggcgggaacacatggaacgacgtcggaggtttgagtttgattttcgagatagagatgatgaacggggttaccgaagggttcgctctggcagtgggagcatagatgatgacagggatagcttgcccgaatggtgcttagaggatgctgaagaagaaatgggtacatttgactcatctggagcattcctttctctaaaaaaagtacagaaagagcctattccagaagagcaggagatggacttccggcctgtggacgaaggggaggagtgctctgactctgagggtagccataatgaagaggccaaagaacccgataagacaaataagaaagaaggagagaaaacagatagagtaggagttgaagctagtgaggaaactccccagacctcatcatcatctgctagaccaggtactccttcagaccatcagtctcaggaagcatcacagtttgagaggaaagatgaaccaaaaactgagcaaacggaaaaagctgaagaggagactcggatggaaaatagtctaccagccaaagtgcccagcagaggggatgaaatggttgctgatgtccagcagcccctgtcgcagattccttcagatacagcctctcctcttctcatacttccacctcctgttcccaatcctagtcctactctccggccagttgaaacaccagttgtaggtgctcctggtatgggcagtgtttccacagaacctgatgatgaagaaggtctcaaacatttggagcagcaagctgagaaaatggtggcttatctccaagacagtgcactagatgatgaaagattggcatcaaaactgcaagagcacagagctaaaggagtgtcgattccattgatgcatgaagcaatgcagaagtggtattacaaagatcctcagggagaaattcaaggtcccttcaataatcaggagatggcagaatggtttcaggcgggctattttactatgtctttattggtgaagagagcgtgtgatgaaagcttccaacctcttggcgatatcatgaaaatgtggggaagggttcccttttctccaggtccagctccccctcctcatatgggagagctggaccaggaacgactgaccaggcagcaagaactcacagccttataccagatgcagcacctgcagtaccagcagtttttaatacaacaacaatatgcacaggttttggcccaacagcagaaagcagcactgtcttcccagcagcagcagcagttggcacttcttcttcaacagtttcagaccttgaagatgagaatatctgatcagaacatcattccctcagtaactaggtctgtgtccgtgccagatactggctctatctgggagcttcagccaacagcttcacagcctacagtttgggaaggtggtagtgtatgggatcttcctctggacaccacgacaccaggccctgccctggaacagcttcagcagctagagaaggccaaagctgcaaagctagagcaagagagaagagaggcagaaatgagggcaaaacgggaagaggaagagcgaaagaggcaggaagaactccgaagacaacaggaggaaattcttcggcgacagcaggaagaagaaaggaaaaggcgagaggaagaagaacttgcccgaaggaaacaggaagaggctctgcgtcgccagcgggagcaagaaattgcattaaggcgacagcgagaagaggaagaaagacagcagcaagaagaagctcttagaagactggaagagaggagaagagaagaggaagaaaggcggaagcaggaagaattgttacgcaaacaggaagaggaggctgcaaaatgggcccgggaagaagaagaagcccagcgtcgattagaggagaaccggctgcggatggaagaggaggcagccagactccggcatgaggaagaagaacggaagagaaaggagctggaggtccagcggcagaaggagttaatgcgccagaggcagcagcagcaagaggctctccggaggttgcagcagcagcagcagcaacaacagctggcgcagatgaagcttccttcttcttcaacgtggggccagcagtccaatacaacagcatgtcagtcccaggccacgctgtcgttggctgaaatccaaaaactagaggaagaacgagaacggcagcttcgagaagagcaaaggcgccagcagagggagttgatgaaagctcttcagcagcagcagcaacagcaacagcagaaactctcaggttgggggaatgtcagcaaaccttcaggtaccacgaaatctcttctggagatccagcaggaagaggccaggcaaatgcaaaagcagcagcagcagcagcagcaacaccagcaaccaaacagagctcgtaacaatacgcattccaacctgcacaccagcattgggaattctgtttggggctctataaatactggtcctcctaaccagtgggcatctgacctagtcagtagtatttggagtaatgctgacactaaaaactccaacatgggattctgggatgatgcagtgaaagaggtgggacctaggaattcaacaaataaaaataaaaacaacgccagtctcagtaaatctgtaggtgtgtctaaccggcagaataagaaagtagaagaagaagaaaagttgctgaagctctttcagggagtaaataaagcccaagatggatttacgcagtggtgtgaacagatgcttcatgcccttaatacggcaaataacttggatgttcccacatttgtttctttcctgaaagaagtagaatctccttatgaggtccatgattatatcagggcctatttaggagatacttctgaggccaaggagtttgccaagcagttccttgagcgccgtgccaaacagaaagccaaccagcagcgtcagcagcagcagctgccacagcagcagcagcagcagccgccacagcagccgccacagcagccacaacagcaggactctgtgtgggggatgaaccacagtacactccattcagtatttcagaccaatcaaagcaacaaccaacaatccaattttgaggctgtgcagagtggcaagaagaagaaaaagcagaagatggtccgagcagatcccagtttattaggattttcagtcaatgcatcatcggagcgactcaacatgggtgaaatcgagacgttggatgactactgagcacctgccagtggactggccatccctctcctgtctgccgactatggagtctccacctttggacacaacacttactcaccatttactctttatcactctgcaacaaatcacagaaccgatcatctcaggctttttcttctggccctttgtgtccaagattctttaatccatttttgttggtgaacatctcagactatagataagtggactggaccctgtgtcttgggggtggcagttgggattactccccaacaaggctgattttaggcagcatgtgttcactgtgctgtgatttcatctactgtctcccagaaagtgtgttgggatcggccattagcagcttgctttctcttgtcactttttttcttctattttgttttttcttcttctttttccccccatcagggcaaatggtctaactggtgcaatcatgaagagagttaatggttaacagacattggccaataacaaaacaccccatggactgtgactcgagtatccaacaggcagtcagagctctcccggtctgaaagttgcattgccactgctaactttgggattgcatcagagaggccctgagtggggttgagatgaggttggtttggtttgatgttacacactcctcacctgttctttctgagtgtcctttctctgaaaggatttatgtttttcttcgttagatagtgacttctgagcaagctgatctcccctggcatgctccaacctgattggacaaaggaagctctatggcctgggagagagactattcttaatttttctttcttacaaaaactgatttttcccataaatatttttacttcagaggactaggaccattttgttttgggcccttctgctgaaaatttgtctcgtttaagaggcagctagaatctttaccatatgtatgaatttgtataatttcatttttggatagggataaacttttgcttctgataaaagcctggaatttcatctggtcctcagagcattgcgtgtgtgtcttgctgtagcccggaaaaggttttgtgtaaagattctgggatggcaagttgtttgccttttctgaaaagagaacatacagaacctgtccatctttaagaccttcatccatggaatctactatacaggaggatgcagtgggctggaggggatgggcgaaaatgggagcaggaagcctggcctggcttctggtcatggcctcctaaaaccttaaacttcaagtagaaatgtactcaagccctatttataaacaaatacttttcctgcctccaccaaacccctacagaacatcacctggaattgccactcacactgggttggagtcattgggcagctgtgcctgtgcgagaggtgctgtggtctgggcagcccctggaaaagcacctttgctgcctgtcattgttgcctgaagaaggctggagttgctctgagagcagtttgggtttggagtattatatttggcttctatttttattattttggatcaccattctccctatcccttcttgcctccctcccttctaaacatgtgtaataactatacagagactgctacaaaattgtatatagtttttggatcaaatagcatgaggggagaggaaaccattaaaagttggggctcctactctcctttgctttgtaaattcaaaagttgggggtgggtaagagggatagttaaaatgtttacaaaactttaggctccctcggaacttttgccagtgtggaggaaaataaaaaagaacttaaataaaatctgattgtattctatctgagtgcacctcttgtactcacctttatggaggctgagttctgcactaaactgttcctcttggtaccatggaaaagctccaagcacccaagacatggaggcagccatggcttctttctctgccagaccacgtagcactggctggttctgtatttgagaatgtagaggtcaaggcagatgtcggtaagtttgaccaaatatcttttctctttttgactctccctttctgctaacatgtggatcagctcctgccctcataccgcagacacacccactctgaacacacccctccagagcttgcccacaccttcccaactagactctgtgtgtgtattcaggaaaaatgaaaataaatttttaaaaattgggagtggaatgggcagattagggggaacaaagtcattttggtcatggaacatccatttgcttaaccaaggagacttgcttctatctgaggaatttcccacttagtagaaagaaacactcttccctgggaatgtttgtttctgtctctctcaatgctactctctttcatattctcatttgctcaccctttcacttagagccaccgttttctcatcccattctatgaacttatttccaaattctcctaactattcctgagttttgaagtatttgttttgtaccctctctgcttccggttttttgtttggttggcttttttggagatagggtcttgtgatgttgcccacactggtctcgagcccctgggctcaagtgatcctcccgcctcagcctcctaaatatctaggactacaggtgtgtgccaccatgcccagctccagttttgttggttgatttgtttaaattcagagatttaaaaacatcccagacaagacagtttgattatggagactgtccccaagcagattggtgtgctggtggctgctgtgcataacagattccctccagattggcttacaacagctacttcattctgtgggtcaggaatttggatagggctcagctgggtggtggttctcctggggtattctttggttgtggtcagatgtcttccagggcatcattcctatgaaggcttaatgaattaggcaagctgtccaagatggctcactcgtgtaatgacagttgacgttggctgtaggctgggaactcagctgggttgatgactggaggagcttatgtgtgtcgtctccggaatggcagcttcagaggagttgaacttaagtggtgattggcttcccccagagaaccaggcagaagctgtgttacctttttgagctcttagctcaggagtcacatagagtcacttctgtgacactgtgttgcatcaaagtagtcacaaacccactcagattcaagagaagggagcatggccctctccctgcaaccccctccccccccgccttgatgaaaggagtgacaaggtcacattggagaagagcttgtggggtgggagatattatagccatttttgggaaatatctgcataaaatgagtttatgcatacatgaaggactttattttcattggctttaagttgagggtttttgtttttgtttttgtattggagatgggttcttactatgttgcccaggctggtctcaaacccctgggctcaagtgatcctgttgcctcagcctcccaagtagctgggactacagctatgtgccagtgcacccagcttgttttttttaaattttaggatagataatggtacttctggctttatttaaataatttttttagggaaaccattgtatttatacatgcatatatttttatatatacaaatatgtatataaaagtatatgttttacattttcaatatatatataatatatacatatatgttatatacgtatatatattgcaccctactttaaaattgactgataacaaaattggtaattttgtataaaatgcccaactgtgcattattaataaaagtatgtgaatgactctaaaaaaa

SEQ ID NO: 38 - protein, 1299 aa, Homo sapiens
MAAETQTLNFGPEWLRALSSGGSITSPPLSPALPKYKLADYRYGREEMLALFLKDNKIPSDLLDKEFLPILQEEPLPPLALVPFTEEEQRNFSMSVNSAAVLRLTGRGGGGTVVGAPRGRSSSRGRGRGRGECGFYQRSFDEVEGVFGRGGGREMHRSQSWEERGDRRFEKPGRKDVGRPNFEEGGPTSVGRKHEFIRSESENWRIFREEQNGEDEDGGWRLAGSRRDGERWRPHSPDGPRSAGWREHMERRRRFEFDFRDRDDERGYRRVRSGSGSIDDDRDSLPEWCLEDAEEEMGTFDSSGAFLSLKKVQKEPIPEEQEMDFRPVDEGEECSDSEGSHNEEAKEPDKTNKKEGEKTDRVGVEASEETPQTSSSSARPGTPSDHQSQEASQFERKDEPKTEQTEKAEEETRMENSLPAKVPSRGDEMVADVQQPLSQIPSDTASPLLILPPPVPNPSPTLRPVETPVVGAPGMGSVSTEPDDEEGLKHLEQQAEKMVAYLQDSALDDERLASKLQEHRAKGVSIPLMHEAMQKWYYKDPQGEIQGPFNNQEMAEWFQAGYFTMSLLVKRACDESFQPLGDIMKMWGRVPFSPGPAPPPHMGELDQERLTRQQELTALYQMQHLQYQQFLIQQQYAQVLAQQQKAALSSQQQQQLALLLQQFQTLKMRISDQNIIPSVTRSVSVPDTGSIWELQPTASQPTVWEGGSVWDLPLDTTTPGPALEQLQQLEKAKAAKLEQERREAEMRAKREEEERKRQEELRRQQEEILRRQQEEERKRREEEELARRKQEEALRRQREQEIALRRQREEEERQQQEEALRRLEERRREEEERRKQEELLRKQEEEAAKWAREEEEAQRRLEENRLRMEEEAARLRHEEEERKRKELEVQRQKELMRQRQQQQEALRRLQQQQQQQQLAQMKLPSSSTWGQQSNTTACQSQATLSLAEIQKLEEERERQLREEQRRQQRELMKALQQQQQQQQQKLSGWGNVSKPSGTTKSLLEIQQEEARQMQKQQQQQQQHQQPNRARNNTHSNLHTSIGNSVWGSINTGPPNQWASDLVSSIWSNADTKNSNMGFWDDAVKEVGPRNSTNKNKNNASLSKSVGVSNRQNKKVEEEEKLLKLFQGVNKAQDGFTQWCEQMLHALNTANNLDVPTFVSFLKEVESPYEVHDYIRAYLGDTSEAKEFAKQFLERRAKQKANQQRQQQQLPQQQQQQPPQQPPQQPQQQDSVWGMNHSTLHSVFQTNQSNNQQSNFEAVQSGKKKKKQKMVRADPSLLGFSVNASSERLNMGEIETLDDY

SEQ ID NO: 39 - DNA, 2794 bp, Homo sapiens
  CDS (341)..(1783)
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 40 - protein, 480 aa, Homo sapiens [sequence omitted]

(19) United States
(12) Patent Application Publication
(10) Pub. No.: US 2012/0022131 A1
(43) Pub. Date: Jan. 26, 2012

(54) BREAST CANCER RELATED GENE RQCD1

(75) Inventors: Yusuke Nakamura, Tokyo (JP); Toyomasa Katagiri, Tokyo (JP); Akira Togashi, Kanagawa (JP)

(73) Assignee: Oncotherapy Science Inc., Kanagawa (JP)

(21) Appl. No.: 13/060,671

(22) PCT Filed: Aug. 13, 2009
(86) PCT No.: PCT/JP2009/003887

§ 371 Date: Feb. 24, 2011

Related U.S. Application Data
(60) Provisional application No. 61/190,389, filed on Aug. 27, 2008.

(51) Int. Cl. A61K 31/713; A61P 35/00; C12Q 1/68; G01N 33/574; C12N 15/113; C12N 15/63

(52) U.S. Cl. 514/44 A; 536/24.1; 435/320.1; 435/6.14; 436/501; 435/6.13; 435/7.1; 435/6.12; 435/6.11

(57) ABSTRACT

The present invention provides methods for detecting and diagnosing cancer, such methods involving the determination of the expression level of the RQCD1, GIGYF1 or GIGYF2 genes. These genes were discovered to discriminate cancer cells from normal cells. Furthermore, the present invention provides methods of screening for therapeutic agents useful in the treatment of cancer and methods for treating cancer. Moreover, the present invention provides siRNAs targeting the RQCD1, GIGYF1 and/or GIGYF2 genes, all of which are suggested to be useful in the treatment of cancer.

### PRIORITY

The present application claims the benefit of U.S. Provisional Application No. 61/190,389, filed on Aug. 27, 2008, the entire content of which is incorporated by reference herein.

The present invention relates to methods for detecting and diagnosing cancer as well as methods for treating and preventing cancer.

### TECHNICAL FIELD

### Background Art

Breast cancer is the most common cancer in women, with estimated new cases of 1.15 million worldwide in 2002 (NPL 1; Parkin D M, et al. (2005). CA Cancer J Clin 55:74-108.). Incidence rates of breast cancer are increasing in most countries, and the increasing rate is much higher in countries where its incidence was previously low (NPL 1; Parkin D M, et al. (2005). CA Cancer J Clin 55:74-108.). While early detection with mammography as well as development of molecular targeted drugs, such as tamoxifen and trastuzumab, have reduced the mortality rate and made the quality of life of the patients better (NPL 2; Navolanic P M and McCubrey J A. (2005). Int J Oncol 27:1341-1344), there remain very limited treatment options for patients with advanced stage disease, particularly those with a hormone-independent tumor. Hence, development of novel drugs to provide better management to such patients is still eagerly expected.

Gene-expression profiles obtained by cDNA microarray analysis have yielded detailed characterization of individual cancers and such information may prove useful in the selection of more appropriate clinical strategies for individual patients, both through development of novel drugs and by providing a basis for personalized treatment (NPL 3; Petricoin E F 3rd, et al. (2002) Nat Genet. 32 Suppl: 474-479.). Through genome-wide expression analysis, a number of genes have been isolated that function as oncogenes in the process of development and/or progression of breast cancers (NPL 4; Park J H, et al. (2006) Cancer Res 66:9186-9195.; NPL 5; Shimo A, et al. (2007) Cancer Sci 98:174-181.; NPL 6; Lin M L, et al. (2007) Breast Cancer Res 9: R17.), synovial sarcomas (NPL 7; Nagayama S, et al. (2004) Oncogene 23:5551-5557.; NPL 8; Nagayama S, et al. (2005) Oncogene 24:6201-6212.), and renal cell carcinomas (NPL 9; Togashi A, et al. (2005) Cancer Res 65:4817-4826., NPL 10; Hirota E, et al. (2006) Int J Oncol 29:799-827.). Such molecules are considered to be candidate targets in the development of new therapeutic modalities.

In an attempt to identify novel molecular targets for breast cancer therapy, detailed gene-expression profiles of breast cancer cells purified by laser microbeam microdissection by means of cDNA microarray were analyzed (NPL 11; Nishidate T, et al. (2004) Int J Oncol 25:797-819, PL 1; WO2005/029067, PL 2; WO2006/016525, PL 3; WO2007/013670). Although some breast cancer markers have been identified through these studies, new therapeutic agents targeting them are still under development. Therefore, the identification of novel genes to be targeted for anticancer therapy remains a goal in the art.

To that end, the RQCD1 (RCD1 required for cell differentiation 1 homolog (S. pombe)) gene previously isolated as a transcriptional cofactor that mediates retinoic acid-induced cell differentiation (NPL 12; Hiroi N, et al. (2002) EMBO J. 21:5235-44) has been identified through microarray analyses as a gene up-regulated in lung cancer and esophagus cancer (PL 4; WO2004/031413, PL 5; WO2007/013665, PL 6; WO/2007/013671). However, to date, no relationship has been established between RQCD1 and breast cancer. Further, RQCD1 has not been confirmed as a suitable target gene for other cancer therapy, only as one of many genes up-regulated therein.

### CITATION LIST

### Non Patent Literature

[NPL 1] Parkin D M, et al. (2005). CA Cancer J Clin 55:74-108
[NPL 2] Navolanic P M and McCubrey J A. (2005). Int J Oncol 27:1341-1344
[NPL 3] Petricoin E F 3rd, et al. (2002) Nat Genet. 32 Suppl:474-479
[NPL 4] Park J H, et al. (2006) Cancer Res 66:9186-9195
[NPL 5] Shimo A, et al. (2007) Cancer Sci 98:174-181
[NPL 6] Lin M L, et al. (2007) Breast Cancer Res 9: R17
[NPL 7] Nagayama S, et al. (2004) Oncogene 23:5551-5557
[NPL 8] Nagayama S, et al. (2005) Oncogene 24:6201-6212
[NPL 9] Togashi A, et al. (2005) Cancer Res 65:4817-4826
[NPL 10] Hirota E, et al. (2006) Int J Oncol 29:799-827 [NPL 11]
[NPL 11] Nishidate T, et al. (2004) Int J Oncol 25:797-819
[NPL 12] Hiroi N, et al. (2002) EMBO J. 21:5235-44

### Patent Literature

[PL 1] WO2005/029067
[PL 2] WO2006/016525
[PL 3] WO2007/013670
[PL 4] WO2004/031413
[PL 5] WO2007/013665
[PL 6] WO/2007/013671

### SUMMARY OF INVENTION

The present invention relates to the discovery of a specific expression pattern of the RQCD1 gene in cancerous cells.

Through the present invention, the RQCD1 gene was revealed to be frequently upregulated in human tumors, in particular, breast tumors. Moreover, since the suppression of the RQCD1 gene by small interfering RNA (siRNA) resulted in growth inhibition and/or cell death of breast cancer cells, this gene may serve as a novel therapeutic target for human breast cancers.

The RQCD1 gene identified herein, as well as its transcription and translation products, find diagnostic utility as a marker for breast cancer and as an oncogene target, the expression and/or activity of which may be altered to treat or alleviate a symptom of cancer. Similarly, by detecting changes in the expression of the RQCD1 gene that arise from exposure to a test compound, various agents for treating or preventing cancer can be identified.

Accordingly, it is an object of the present invention to provide a method for diagnosing or determining a predisposition to breast cancer in a subject by determining the expression level of the RQCD1 gene in a subject-derived biological sample, such as tissue sample. As increase in the level of expression of the gene as compared to a normal control level indicates that the subject suffers from or is at risk of developing breast cancer.

In the context of the present invention, the phrase “control level” refers to the expression level of the RQCD1 gene detected in a control sample and encompasses both a normal control level and a cancer control level. A control level can be a single expression pattern derived from a single reference population or the average calculated from a plurality of expression patterns. Alternatively, the control level can be a database of expression patterns from previously tested cells. The phrase “normal control level” refers to a level of the RQCD1 gene expression detected in a normal healthy individual or in a population of individuals known not to be suffering from cancer. A normal individual is one with no clinical symptom of breast cancer. A normal control level can be determined using a normal cell obtained from a non-cancerous tissue. A “normal control level” may also be the expression level of the RQCD1 gene detected in a normal healthy tissue or cell of an individual or population known not to be suffering from breast cancer. On the other hand, the phrase “cancer control level” refers to an expression level of the RQCD1 gene detected in the cancerous tissue or cell of an individual or population suffering from breast cancer.

An increase in the expression level of the RQCD1 gene detected in a sample as compared to a normal control level indicates that the subject (from which the sample has been obtained) suffers from or is at risk of developing breast cancer.

Alternatively, the expression level of the RQCD1 gene in a sample can be compared to cancer control level of the RQCD1 gene. A similarity between the expression level of a sample and the cancer control level indicates that the subject (from which the sample has been obtained) suffers from or is at risk of developing cancer.

Herein, gene expression levels are deemed to be “altered” when the gene expression increases by, for example, 10%, 25%, or 50% from, or at least 0.1 fold, at least 0.2 fold, at least 0.5 fold, at least 2 fold, at least 5 fold, or at least 10 fold or more compared to a control level. The expression level of the RQCD1 gene can be determined by the hybridization intensity of nucleic acid probes to gene transcripts in a sample.

In the context of the present invention, subject-derived tissue samples may be any tissues obtained from test subjects, e.g., patients known to have or suspected of having cancer. For example, tissues may include epithelial cells. More particularly, tissues may be cancerous epithelial cells.

It is yet another object of the present invention to provide methods for identifying compounds that inhibit the expression or activity of the RQCD1 protein, by contacting a test cell expressing the RQCD1 protein with test compounds and determining the expression level of the RQCD1 gene or the activity of the gene product, the RQCD1 protein. The test cell may be an epithelial cell, such as cancerous epithelial cell. A decrease in the expression level of the gene or the activity of its gene product as compared to a control level in the absence of the test compound indicates that the test compound may be used to reduce symptoms of breast cancer.

The present invention also provides a kit that includes at least one detection reagent that binds to a transcription or translation product of the RQCD1 gene.

Therapeutic methods of the present invention include methods for treating or preventing breast cancer in a subject including the step of administering an antisense composition to the subject. In the context of the present invention, the antisense composition reduces the expression of the RQCD1 gene. For example, the antisense compositions may contain a nucleotide that is complementary to the RQCD1 gene sequence. Alternatively, the present methods may include the step of administering siRNA composition to the subject. In the context of the present invention, the siRNA composition reduces the expression of the RQCD1 gene. In yet another method, the treatment or prevention of breast cancer in a subject may be carried out by administering a ribozyme composition to the subject. In the context of the present invention, the nucleic acid-specific ribozyme composition reduces the expression of the RQCD1 gene.

To that end, the present inventors confirmed inhibitory effects of siRNAs for the RQCD1 gene. In particular, the inhibition of cell proliferation of cancer cells by the siRNAs is demonstrated in the Examples section. The data herein support the utility of the RQCD1 gene as a preferred therapeutic target for breast cancer. Thus, the present invention also provides double-stranded molecules that serve as siRNAs against the RQCD1 gene as well as vectors expressing the double-stranded molecules.

It is a further object of the present invention to provide a method of screening for a candidate compound for treating or preventing breast cancer, said method including the steps of:

(a) contacting a GIGYF1 and/or GIGYF2 polypeptide or functional equivalent thereof with an RQCD1 polypeptide or functional equivalent thereof in the presence of a test compound;

(b) detecting the binding between the polypeptides of the step (a); and

(c) selecting the test compound that inhibits the binding between the GIGYF1 or GIGYF2 and RQCD1 polypeptides.

The present invention further provides a kit for screening for a compound for treating or preventing breast cancer, said kit including components of:

(a) a GIGYF1 and/or GIGYF2 polypeptide or functional equivalent thereof, and

(b) an RQCD1 polypeptide or functional equivalent thereof.

The present invention further provides a method of treating or preventing breast cancer in a subject that includes the step of administering to said subject an siRNA composition including an siRNA that reduces the expression of GIGYF1 and/or GIGYF2 gene, wherein the siRNA includes the nucleotide sequence of SEQ ID NO: 32 or 33, in the sense strand.

One advantage of the methods described herein is that the disease is identified prior to detection of overt clinical symptoms of breast cancer. Other features and advantages of the invention will be apparent from the following detailed description, and from the claims.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

Various aspects and applications of the present invention will become apparent to the skilled artisan upon consideration of the brief description of the figures and the detailed description of the present invention and its preferred embodiments that follows:

FIG. 1 depicts the expression pattern of RQCD1 in the clinical breast cancer cells and normal human organs assayed in Example 1. Part (A) depicts the results of semiquantitative RT-PCR, confirming the over-expression of RQCD1 in breast cancer cells. Amplified cDNA products from 12 clinical samples (#42, #102, #247, #252, #302, #473, #478, #502, #552, #646, #769 and #779) were presented in comparison with that from microdissected normal mammary ductal cells or that from normal organs including lung, heart, liver, kidney and mammary gland. Beta-actin (ACTB) served as an internal control. Part (B) depicts the results of Northern blot analysis of breast cancer cell lines. Radioisotope-labeled probe designed for RQCD1-specific sequence was detected at the level of 3.5 kb corresponding to full length mRNA of RQCD1. All of the 11 breast cancer cell lines showed up-regulated expression rather than normal tissues except for testis. Part (C) depicts the tissue-specific distribution of RQCD1 by multiple tissue Northern blot, showing positive signal from testis, but no or very weak signal was detected from other tissues.

FIG. 2 depicts the immunocytochemistry results of exogenously introduced RQCD1 assayed in Example 1. 24 h after transfection of pCAGGS-HA-RQCD1, HEK293, HBC4 and BT549 were applied to immunocytochemistry with anti-HA antibody (red) and DAPI (blue). In all of three cell lines, exogenous RQCD1 was expressed in cytoplasm and nuclear (scale bar=25 mm).

FIG. 3 depicts the effect of RQCD1 knockdown by siRNA on the growth of breast cancer cells in Example 1. Part (A) depicts the RQCD1 knockdown results arising when either of two siRNA expression vectors specific to RQCD1 transcript (#1 or #2) and a mock siRNA expression vector (without target sequence) as a control are transfected into HBC4 and BT549. Knockdown effect on RQCD1 transcript was examined by semiquantitative RT-PCR, with beta actin as a control. Transfection with siRNA #1 or #2 showed significant knockdown effect. Part (B) depicts the results of a colony formation assay wherein transfection with #1 or #2 vector resulted in a drastic reduction in the surviving cell number compared with mock vector transfected cells. Part (C) depicts the drastic decrease in cell number of RQCD1 knock downed cells also quantitatively confirmed by WST-1 proliferation assay. Part (D) depicts the effect of an siRNA vector with 3-base mismatch in #1 siRNA target sequence transfected to HBC4. 3-base mismatch siRNA on attenuation of RQCD1 expression. Part (E) depicts the results of a colony formation assay wherein transfection of 3-base mismatch siRNA vector showed no effect on the cell number. Part (F) depicts the quantitative evaluation of cell proliferation by WST-1 proliferation assay wherein a 3-base mismatch siRNA vector had no effect on cell proliferation.

FIG. 4 depicts the stable overexpression of RQCD1 promoted cell growth of HEK293. Part (A) depicts the results of Western blot analysis of 3 clones respectively from RQCD1 stable cell lines (stable-1, -2 and -3) or mock stable cell lines (mock-1, -2 and -3) assayed in Example 1. Expression level of introduced RQCD1 was validated with anti-HA tag antibody. Anti-beta actin antibody served as a loading control. Part (B) depicts the growth rate measured by MTT assay wherein three RQCD1 stable clones showed more rapid cell growth than three mock stable clones. X-axis, day points after seeding; Y-axis, relative absorbance score of WST-1 proliferation assay by comparison with the absorbance value of day 1 as a control. Points, average; bars, SE. This assay was examined in triplicate. Part (C) depicts the rapid growth multilayer-growth of these three HEK293-RQCD1 cells observed after they reached at the confluence phase, indicating loss of the contact inhibition mechanism by RQCD1 introduction into HEK293 cells.

FIG. 5 depicts expression levels of RQCD1 in clinical breast cancer samples, breast cancer cell lines and human normal tissues in Example 2. Part (A) depicts the results of semi-quantitative RT-PCR for 12 breast cancer clinical samples and human normal tissues including normal mammary ductal cells, whole mammary gland, lung, heart, liver, and kidney. Beta-actin (ACTB) was used as an internal control. Part (B) depicts the results of Northern blotting analysis for 11 breast cancer cell lines and human normal multiple tissues with a [alpha³²P]-dCTP-labeled RQCD1 cDNA fragment as a probe. For breast cancer cell lines and human normal mammary gland, 1 microgram each of mRNA was applied to each lane. For human multiple normal tissues, 2 microgram each of mRNA was applied to each lane. Part (C) depicts the results of Western blotting of RQCD1 for breast cancer cell lines and human normal tissues with purified anti-RQCD1 polyclonal antibody. Five-microgram each of total protein was applied to each lane. Equal amount of loading proteins were confirmed by staining nitrocellulose membrane with Ponceou S.

FIG. 6 depicts expression levels of RQCD1 in clinical breast cancer samples, breast cancer cell lines and human normal tissues in Example 2. Part (D) depicts the results of immunocytostaining of RQCD1 in BT-549 cells. RQCD1 was probed with anti-RQCD1 polyclonal antibody and Alexa-488 (green), and cell nuclear was counterstained with DAPI (blue). Scale bar indicates 20 micrometer.

FIG. 7 depicts the effect of RQCD1 on cell growth in Example 2. Part (A) depicts the effect of RNAi on growth of breast caner cell lines. shRNA expression vectors specific to RQCD1 transcript (#1 or #2) and a mock shRNA expression vector (mock) were transfected into BT-549 and HBC-4 cells, respectively. Knockdown effect of RQCD1 was examined by semi-quantitative RT-PCR and western-blot analyses. Cell proliferation assay and colony formation assay were performed for evaluation of knockdown effect on cell growth. Columns; average of three independent experiments, bars; +/−SE. *; P=0.002 and **; P=0.004 by Student's t-test compared to mock transfected cells.

FIG. 8 depicts the effect of RQCD1 on cell growth in Example 2. Part (B) depicts the growth rate of HEK293 cells in which RQCD1 was stably expressing. Western blotting was performed for three independent clones of HEK293 derivative cells expressing RQCD1 (stable-1, -2 and -3) and those transfected with mock vector (mock-1, -2 and -3). Each cell line was seeded at 0.4×10⁵ cells to 6-well plate, and 7 days after seeding, cell proliferation assay was performed. X-axis; day points after seeding, Y-axis; fold increase in cell number from the first day. Points; an average of three independent experiments, bars; +/−SE, *; P<0.0001 by Student's t-test.

FIG. 9 depicts the interaction and co-localization of RQCD1 with GIGYF1 or GIGYF2 in Example 2. Part (A) depicts the results of a co-immunoprecipitation assay for Flag-RQCD1, and HA-GIGYF1 or HA-GIGYF2 in HEK293T cells. Immunoprecipitation by anti-Flag or anti-HA agarose was performed at 36 hours after the co-transfection of Flag-RQCD1, and HA-GIGYF1 or HA-GIGYF2. Precipitated proteins were competitively eluted with 3xFlag peptide or HA peptide. Subsequently, western blotting was performed for detection of input controls and peptide-eluted samples. Part (B) depicts the expression levels of GIGYF1 and GIGYF2 in breast cancer cell lines and normal mammary gland examined by semi-quantitative RT-PCR. ACTB served as an internal control. Part (C) depicts the immunocytostaining of exogenously-expressed HA-GIGYF1 and HA-GIGYF2 in BT-549 cells. Thirty-six hours after transfection to BT-549, the cells were fixed, and HA-GIGYF1 (red), HA-GIGYF2 (red), and endogenous RQCD1 (green) were immunostained. Nuclei were counterstained with DAPI (blue). A scale bar indicates 10 micrometer.

FIG. 10 depicts effect on Akt activity by knockdown of RQCD1, GIGYF1 or GIGYF2 in breast cancer cells in Example 2. Part (A) depicts the effect on Akt activity in breast cancer cells under presence or absence of serum treatment. MCF-10A, BT-549, HBC-5, and HCC-1937 cells were cultured in each appropriate culture medium with or without FBS and growth factors for 24 h, and then Akt activity was analyzed by western blotting with anti-Akt and anti-phospho-Akt (Ser 473) antibodies. Part (B) depicts the total amount of Akt and its phosphorylation level examined by western blotting with anti-Akt and anti-phospho-Akt (Ser 473) antibodies at 72 h after transfection of siRNA against RQCD1 (left panels), GIGYF1 or GIGYF2 (right panels) in BT-549 cells. Cells were cultured in serum-depleted medium for 24 h before harvesting. Knockdown effects of GIGYF1 and GIGYF2 were confirmed by semi-quantitative RT-PCR. Part (C) depicts the total amount of Akt and its phosphorylation level examined by western blotting with anti-Akt and anti-phospho-Akt (Ser 473) antibodies at 72 h after knockdown of RQCD1 in HBC-5 (left panels) and HCC-1937 cells (right panels). Cells were cultured in serum-depleted medium for 24 h before harvesting.

FIG. 11 depicts effect on Akt activity by knockdown of RQCD1, GIGYF1 or GIGYF2 in breast cancer cells in Example 2. Part (D) depicts the Akt activity in each breast cancer cell line quantitatively evaluated by ratio of phospho-Akt (Ser 473)/total Akt signal intensity by densitometric analysis of ECL signals using Image J (Abramoff M D, Magelhaes P J and Ram S J, Image Processing with Image J. Biophotonics International 11: 36-42, 2004). Assays were carried out three times. Columns; average of three independent analysis, bars; +/−SE, *; P<0.05 by Student's t-test, compared to si-EGFP treated cells.

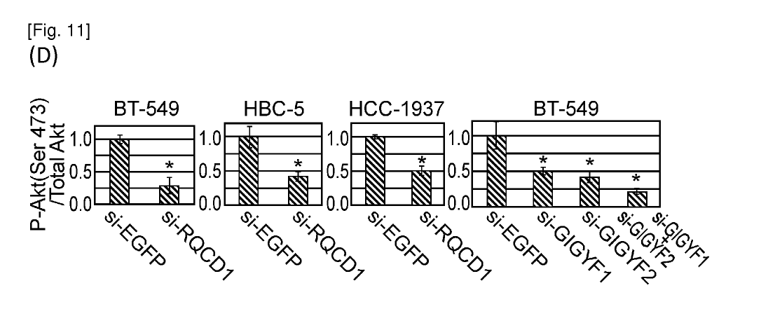

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

Unless otherwise defined, all technical and scientific terms used herein have the same meaning as commonly understood by one of ordinary skill in the art to which this invention belongs. In case of conflict, the present specification, including definitions, will control.

### DEFINITIONS

The words “a”, “an”, and “the” as used herein mean “at least one” unless otherwise specifically indicated. The terms “isolated” and “purified” used in relation with a substance (e.g., polypeptide, antibody, polynucleotide, etc.) indicates that the substance is substantially free from at least one substance that may else be included in the natural source. Thus, an isolated or purified antibody refers to antibodies that are substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies and polypeptides of the present invention are isolated or purified. An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotides”, “oligonucleotide”, “nucleotides”, “nucleic acids”, and “nucleic acid molecules” are used interchangeably unless otherwise specifically indicated and are similarly to the amino acids referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules may be composed of DNA, RNA or a combination thereof.

Unless otherwise defined, the terms “cancer” refers to cancers over-expressing the RQCD1 gene, in particular, breast cancer.

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes an RQCD1 sense nucleic acid sequence (also referred to as “sense strand”), an RQCD1 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules including complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, including a first and second regions complementary to one another, i.e., sense and antisense strands. The degree of complementarity and orientation of the regions is sufficient such that base pairing occurs between the regions, the first and second regions is joined by a loop region, the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotied composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules including complementary sequences to one another and that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, including a first and second regions complementary to one another, i.e., sense and antisense strands. The degree of complementarity and orientation of the regions is sufficient such that base pairing occurs between the regions, the first and second regions is joined by a loop region, the loop results from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the context of the present invention, examples of isolated nucleic acid include DNA, RNA, and derivatives thereof.

The present invention is based in part on the discovery of elevated expression of the RQCD1 gene in cells from patients of breast cancers. The nucleotide sequence of the human RQCD1 gene is shown in SEQ ID NO: 10 and is also available as GenBank Accession No. NM_{—}005444. Herein, the RQCD1 gene encompasses the human RQCD1 gene as well as those of other animals including but not limited to non-human primate, mouse, rat, dog, cat, horse, and cow, and further includes allelic mutants and genes found in other animals as corresponding to the RQCD1 gene.

The nucleotide sequence of human GIGYF1 gene and GIGYF2 gene are shown in SEQ ID NO: 35 and SEQ ID NO: 37 respectively and are also available as GenBank Accession No. NM_{—}022574.4 and No. NM_{—}015575.3 respectively. Herein, the GIGYF1 gene and GIGYF2 gene encompass the human GIGYF1 gene and GIGYF2 gene as well as those of other animals including but not limited to non-human primate, mouse, rat, dog, cat, horse, and cow, and further include allelic mutants and genes found in other animals as corresponding to the GIGYF1 gene and GIGYF2 gene.

The amino acid sequence encoded the human RQCD1 gene is shown in SEQ ID NO: 11 and is also available as GenBank Accession No. NP_{—}005435. In the context of the present invention, the polypeptide encoded by the RQCD1 gene is referred to as “RQCD1”, and sometimes as “RQCD1 polypeptide” or “RQCD1 protein”.

The amino acid sequence encoded in the human GIGYF1 gene and GIGYF2 gene is shown in SEQ ID NO: 36 and SEQ ID NO: 38 and is also available as GenBank Accession No. NP_{—}072096.2 and NP_{—}056390.2 respectively. In the context of present invention, the polypeptide encoded by the GIGYF1 gene and GIGYF2 gene is referred to as “GIGYF1” and “GIGYF2”, and sometimes as “GIGYF1 polypeptide” and “GIGYF2 polypeptide”, or “GIGYF1 protein” and “GIGYF2 protein”.

According to an aspect of the present invention, functional equivalents are also included in the RQCD1 protein, the GIGYF protein and the GIGYF protein, respectively. Herein, a “functional equivalent” of a protein is a polypeptide that has a biological activity equivalent to the protein. Namely, any polypeptides that retain the biological ability of the RQCD1 protein, the GIGYF1 protein or the GIGYF2 protein may be used as such functional equivalents of each protein in the present invention.

The biological activities of the RQCD1 protein include, for example, regulating activity for cell differentiation, cancer cell proliferation activity, GIGYF1- or GIGYF2-binding activity, and Akt phosphorylation activity.

The GIGYF1 (GRB10 interacting GYF protein 1) gene and the GIGYF2 (GRB10 interacting GYF protein 2) gene have been identified as genes transiently linked to IGF-I receptors by the Grb10 adapter protein following IGF-I stimulation (Gionannone B, et al. (2003) J BIol CHem 34:31564-31573). The GIGYF1 protein and GIGYF2 protein were demonstrated herein to bind to the RQCD1 protein to and involve Akt phosphorylation as well as the RQCD1 protein. Therefore, the biological activities of the GIGYF1 protein and the GIGYF2 protein include, for example, RQCD1-binding activity and Akt phosphorylation activity.

Such functional equivalents include those wherein one or more amino acids are substituted, deleted, added, or inserted to the natural occurring amino acid sequence of the RQCD1 protein, the GIGYF1 protein or the GIGYF2 protein. Alternatively, the polypeptide may be one that includes an amino acid sequence having at least about 80% homology (also referred to as sequence identity) to the sequence of the respective proteins, more preferably at least about 90% to 95% homology, even more preferably 96% to 99% homology. In other embodiments, the polypeptide can be encoded by a polynucleotide that hybridizes under stringent conditions to the naturally occurring nucleotide sequence of the RQCD1 gene, the GIGYF1 gene or GIGYF2 gene.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will vary in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10 degrees C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions can be as following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to any of the human RQCD1 protein, the GIGYF1 protein or GIGYF2 protein can be routinely selected by a person skilled in the art. For example, hybridization may be performed by conducting prehybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

In general, modifications of one, two, or more amino acids in a protein will not influence the function of the protein. In fact, mutated or modified proteins (i.e., peptides composed of an amino acid sequence in which one, two, or several amino acid residues have been modified through substitution, deletion, insertion and/or addition) have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence that alter a single amino acid or a small percentage of amino acids or those considered to be a “conservative modification” wherein the alteration of a protein results in a protein with similar functions, are acceptable in the context of the instant invention. Thus, in one embodiment, the peptides of the present invention may have an amino acid sequence wherein one, two or even more amino acids are added, inserted, deleted, and/or substituted in the human RQCD1, GIGYF1 and GIGYF2 sequences.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 5 or 6 amino acids or less, and even more preferably 3 or 4 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:
1) Alanine (A), Glycine (G);
2) Aspartic acid (d), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cystein (C), Methionine (M) (see, e.g., Creighton, Proteins 1984).

Such conservatively modified polypeptides are included in the present RQCD1 protein, the GIGYF1 protein and the GIGYF2 protein. However, the present invention is not restricted thereto and the RQCD1 protein, the GIGYF1 protein and the GIGYF2 protein includes non-conservative modifications so long as they retain at least one biological activity of the RQCD1 protein, the GIGYF1 protein and the GIGYF2 protein respectively. Furthermore, the modified proteins do not exclude polymorphic variants, interspecies homologues, and those encoded by alleles of these proteins.

Moreover, the RQCD1 gene, the GIGYF1 gene and the GIGYF2 gene of the present invention encompasses polynucleotides that encode such functional equivalents of the RQCD1 protein, the GIGYF1 protein and the GIGYF2 protein respectively.

I. Diagnosing Cancer:

I-1. Method for Diagnosing Cancer or a Predisposition for Developing Cancer

The expression of the RQCD1 gene was found to be specifically elevated in patients with cancer, more particularly, in breast cancer. Accordingly, the genes identified herein as well as their transcription and translation products find diagnostic utility as a marker for breast cancer and by measuring the expression of the RQCD1 gene in a cell sample, breast cancer can be diagnosed. More particularly, the present invention provides a method for detecting, diagnosing and/or determining the presence of or a predisposition for developing cancer in a subject by determining the expression level of the RQCD1 gene in the subject. Preferred cancers to be diagnosed by the present method include breast cancer.

Further, according to the present invention, the GIGYF1 and the GIGYF2 gene were identified as the genes which gene products were interacted with the RGCD1 protein. The GIGYF1 gene and the GIGYF2 gene were also found to be specifically elevated in cancer cells, particularly breast cancer cells. Accordingly, the present invention also provides method for detecting, diagnosing and/or determining the presence of or a predisposition for developing cancer in a subject by determining the expression level of the GIGYF1 gene and the GIGYF2 gene in the subject. Alternatively, the present invention provides a method for detecting the presence of a cancer cell in a subject-derived breast tissue sample, said method including the step of determining the expression level of the RQCD1, GIGYF1, or GIGYF2 gene in a subject-derived biological sample, wherein an increase in said expression level as compared to a normal control level of said gene indicates the presence or suspicion of cancer cell in the tissue.

Such result may be combined with additional information to assist a doctor, nurse, or other practitioner to diagnose that a subject suffers from the disease or is predisposed to developing the disease. Alternatively, the present invention may provide a doctor with useful information to diagnose that the subject suffers from the disease. For example, according to the present invention, when the suspicion or doubt of the presence of cancer cells in the tissue obtained from a subject is indicated, clinical decisions would be made by a doctor with consideration of this observation and another aspect including the pathological finding of the tissue, levels of known tumor marker(s) in blood, or clinical course of the subject, etc. Some blood tumor markers for diagnostic purpose of breast cancer are well known. For example, carbohydrate antigen 125 (CA125), carbohydrate antigen 15-3 (CA15-3), or carcinoembryonic antigen (CEA) is preferable blood tumor marker for breast cancer. Namely, in a particular embodiment, according to the present invention, an intermediate result for examining the condition of a subject may also be provided.

In another embodiment, the present invention provides a method for detecting a diagnostic marker of cancer, said method including the step of detecting the expression of the RQCD1, GIGYF1, or GIGYF2 gene in a subject-derived biological sample as a diagnostic marker of cancer. Preferable cancers to be diagnosed by the present method include breast cancer.

In the context of the present invention, the term “diagnosing” is intended to encompass predictions and likelihood analysis. The present method is intended to be used clinically in making decisions concerning treatment modalities, including therapeutic intervention, diagnostic criteria such as disease stages, and disease monitoring and surveillance for cancer. According to the present invention, an intermediate result for examining the condition of a subject may also be provided. Such intermediate result may be combined with additional information to assist a doctor, nurse, or other practitioner to determine that a subject suffers from the disease. Alternatively, the present invention may be used to detect cancerous cells in a subject-derived tissue, and provide a doctor with useful information to diagnose that the subject suffers from the disease.

A subject to be diagnosed by the present method is preferably a mammal. Exemplary mammals include, but are not limited to, human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of the RQCD1 gene, the GIGYF1 gene or the GIGYF2 gene. The biological samples include, but are not limited to, bodily tissues and fluids, such as blood, sputum, and urine. Preferably, the biological sample contains a cell population including an epithelial cell, more preferably a cancerous breast epithelial cell or a breast epithelial cell derived from tissue suspected to be cancerous. Further, if necessary, the cell may be purified from the obtained bodily tissues and fluids, and then used as the biological sample.

According to the present invention, the expression level of the RQCD1 gene, the GIGYF1 gene or the GIGYF2 gene is determined in the subject-derived biological sample. The expression level can be determined at the transcription (nucleic acid) product level, using methods known in the art. For example, the mRNA of the RQCD1 gene, the GIGYF1 gene or the GIGYF2 gene may be quantified using probes by hybridization methods (e.g., Northern hybridization). The detection may be carried out on a chip or an array. The use of an array is preferable for detecting the expression level of a plurality of genes (e.g., various cancer specific genes) including the present RQCD1 gene, the GIGYF1 gene or the GIGYF2 gene. Those skilled in the art can prepare such probes utilizing the sequence information of the RQCD1 gene, the GIGYF1 gene or the GIGYF2 gene. For example, the cDNA of the RQCD1 gene, the GIGYF1 gene or the GIGYF2 gene may be used as the probes. If necessary, the probe may be labeled with a suitable label, such as dyes and isotopes, and the expression level of the gene may be detected as the intensity of the hybridized labels.

Furthermore, the transcription product of the RQCD1 gene, the GIGYF1 gene or the GIGYF2 gene may be quantified using primers by amplification-based detection methods (e.g., RT-PCR). Such primers can also be prepared based on the available sequence information of the gene. For example, the primers (SEQ ID NOs: 3, 4, 6, 7, 10, 11, 12 and 13 for RQCD1; SEQ ID NOs: 14 and 15 for GIGYF1; SEQ ID NOs: 16 and 17 for GIGYF2) used in the Example may be employed for the detection by RT-PCR, but the present invention is not restricted thereto.

Specifically, a probe or primer used for the present method hybridizes under stringent, moderately stringent, or low stringent conditions to the mRNA of the RQCD1 gene, the GIGYF1 gene or the GIGYF2 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tₘ) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of the RQCD1 protein, the GIGYF1 protein or the GIGYF2 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment retains the binding ability to the RQCD1 protein, the GIGYF1 protein or the GIGYF2 protein. Methods to prepare these kinds of antibodies for the detection of proteins are well known in the art, and any method may be employed in the present invention to prepare such antibodies and equivalents thereof.

As another method to detect the expression level of the RQCD1 gene, the GIGYF1 gene or the GIGYF2 gene based on its translation product, the intensity of staining may be observed via immunohistochemical analysis using an antibody against the RQCD1 protein, the GIGYF1 protein or the GIGYF2 protein. Namely, the observation of strong staining indicates increased presence of the protein and at the same time high expression level of the RQCD1 gene, the GIGYF1 gene or the GIGYF2 gene.

Furthermore, the translation product may be detected based on its biological activity. Specifically, the RQCD1 protein was demonstrated herein to be involved in the growth of cancer cells. Thus, the cancer cell growth promoting ability of the RQCD1 protein may be used as an index of the RQCD1 protein existing in the biological sample.

Moreover, in addition to the expression level of the RQCD1 gene, the GIGYF1 gene or the GIGYF2 gene, the expression level of other cancer-associated genes, for example, genes known to be differentially expressed in breast cancer, may also be determined to improve the accuracy of the diagnosis. Alternatively, the combination of the expression level among the RQCD1 gene, the GIGYF1 gene and the GIGYF2 gene may be determined for more accurate diagnosis.

The expression level of cancer marker genes including the RQCD1 gene, the GIGYF1 gene or the GIGYF2 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of the RQCD1 gene, the GIGYF1 gene or the GIGYF2 gene in samples from subjects whose disease state are known. Furthermore, the control level can be a database of expression patterns from previously tested cells. Moreover, according to an aspect of the present invention, the expression level of the RQCD1 gene, the GIGYF1 gene or the GIGYF2 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of the RQCD1 gene, the GIGYF1 gene or the GIGYF2 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known to be non-cancerous is called “normal control level”. On the other hand, if the control level is determined from a cancerous biological sample, it will be called “cancerous control level”.

When the expression level of the RQCD1 gene, the GIGYF1 gene or the GIGYF2 gene are increased compared to the normal control level or is similar to the cancerous control level, the subject may be diagnosed to be suffering from or at a risk of developing cancer. Furthermore, in case where the expression levels of multiple cancer-related genes are compared, a similarity in the gene expression pattern between the sample and the reference that is cancerous indicates that the subject is suffering from or at a risk of developing cancer.

Difference between the expression levels of a test biological sample and the control level can be normalized to the expression level of control nucleic acids, e.g. housekeeping genes. Genes whose expression levels are known not to differ depending on the cancerous or non-cancerous state of the cell. Exemplary control genes include, but are not limited to, beta actin, glyceraldehyde 3 phosphate dehydrogenase, and ribosomal protein P1.

Furthermore, the present invention provides the use of the RQCD1 gene, the GIGYF1 gene or the GIGYF2 gene as cancerous markers. These genes are particularly useful for breast cancerous markers. For example, it can be determined whether a biological sample contains cancerous cells, especially breast cancerous cells, by detecting the expression level of the RQCD1 gene, the GIGYF1 gene or the GIGYF2 gene as cancerous markers. Specifically, increasing the expression level of the RQCD1 gene, the GIGYF1 gene or the GIGYF2 gene in a biological sample as compared to a normal control level indicates that the biological sample contains cancerous cells. The expression level can be determined by detecting the transcription or translation products of these marker genes as described above. The translation product may be determined as the biological activity.

I-2. Assessing Efficacy of Cancer Treatment

The RQCD1 gene, the GIGYF1 gene and the GIGYF2 gene differentially expressed between normal and cancerous cells also allow for the course of treatment of cancers to be monitored, and the above-described method for diagnosing cancer can be applied for assessing the efficacy of a treatment on cancer. Specifically, the efficacy of a treatment on cancer can be assessed by determining the expression level of the RQCD1 gene, the GIGYF1 gene or the GIGYF2 gene in a cell(s) derived from a subject undergoing the treatment. If desired, test cell populations are obtained from the subject at various time points, before, during, and/or after the treatment. The expression level of the RQCD1 gene, the GIGYF1 gene or the GIGYF2 gene can be, for example, determined following the method described above under the item of ‘I-1. Method for diagnosing cancer or a predisposition for developing cancer’. In the context of the present invention, it is preferable that the control level to which the detected expression level is compared be obtained from the RQCD1 gene, the GIGYF1 gene or the GIGYF2 gene expression in a cell(s) not exposed to the treatment of interest.

If the expression level of the RQCD1 gene, the GIGYF1 gene or the GIGYF2 gene is compared to a control level that is obtained from a normal cell or a cell population containing no cancer cell, a similarity in the expression level indicates that the treatment of interest is efficacious and a difference in the expression level indicates less favorable clinical outcome or prognosis of that treatment. On the other hand, if the comparison is conducted against a control level that is obtained from a cancer cell or a cell population containing a cancer cell(s), a difference in the expression level indicates efficacious treatment, while a similarity in the expression level indicates less favorable clinical outcome or prognosis.

Furthermore, the expression levels of the RQCD1 gene, the GIGYF1 gene or the GIGYF2 gene before and after a treatment can be compared according to the present method to assess the efficacy of the treatment. Specifically, the expression level detected in a subject-derived biological sample after a treatment (i.e., post-treatment level) is compared to the expression level detected in a biological sample obtained prior to treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the forming of tumor or retards, prevents, or alleviates at least one clinical symptom of cancer. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice.

To the extent that the methods and compositions of the present invention find utility in the context of “prevention” and “prophylaxis”, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g., reducing the proliferation and metastasis of tumors.

The treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

II. Kits:

The present invention also provides reagents for detecting cancer, i.e., reagents that can detect the transcription or translation product of the RQCD1 gene, the GIGYF1 gene or the GIGYF2 gene. Examples of such reagents include those capable of:

(a) detecting mRNA of the RQCD1 gene, the GIGYF1 gene or the GIGYF2 gene;

(b) detecting the RQCD1 protein, the GIGYF1 protein or the GIGYF2 protein; and/or

(c) detecting the biological activity of the RQCD1 protein, the GIGYF1 protein or the GIGYF2 protein in a subject-derived biological sample.

Suitable reagents include nucleic acids that specifically bind to or identify a transcription product of the RQCD1 gene, the GIGYF1 gene or the GIGYF2 gene. For example, a nucleic acid that specifically binds to or identifies a transcription product of the RQCD1 gene includes, for example, oligonucleotides (e.g., probes and primers) having a sequence that is complementary to a portion of the RQCD1 gene, the GIGYF1 gene or the GIGYF2 gene transcription product. Such oligonucleotides are exemplified by primers and probes that are specific to the mRNA of the gene of interest and may be prepared based on methods well known in the art. Alternatively, antibodies can be exemplified as reagents for detecting the translation product of the genes. The probes, primers, and antibodies described above under the item of ‘I-1. Method for diagnosing cancer or a predisposition for developing cancer’ can be mentioned as suitable examples of such reagents. These reagents may be used for the above-described diagnosis of cancer. The assay format for using the reagents may be Northern hybridization or sandwich ELISA, both of which are well-known in the art.

The detection reagents may be packaged together in the form of a kit. For example, the detection reagents may be packaged in separate containers. Furthermore, the detection reagents may be packaged with other reagents necessary for the detection. For example a kit may include a nucleic acid or antibody (either bound to a solid matrix or packaged separately with reagents for binding them to the matrix) as the detection reagent, a control reagent (positive and/or negative), and/or a detectable label. A kit of the present invention may further include other materials desirable from a commercial and user standpoint, including buffers, diluents, filters, needles, syringes. These reagents and such may be retained in a container with a label. Suitable containers include bottles, vials, and test tubes. The containers may be formed from a variety of materials, such as glass or plastic. Instructions (e.g., written, tape, VCR, CD-ROM, etc.) for carrying out the assay may also be included in the kit.

Although the present kit is suited for the detection and diagnosis of breast cancer, it may also be useful in assessing the prognosis of cancer and/or monitoring the efficacy of a cancer therapy.

As an aspect of the present invention, the reagents for detecting cancer may be immobilized on a solid matrix, such as a porous strip, to form at least one site for detecting cancer. The measurement or detection region of the porous strip may include a plurality of sites, each containing a detection reagent (e.g., nucleic acid). A test strip may also contain sites for negative and/or positive controls. Alternatively, control sites may be located on a separate strip from the test strip. Optionally, the different detection sites may contain different amounts of immobilized detection reagents (e.g., nucleic acid), i.e., a higher amount in the first detection site and lesser amounts in subsequent sites. Upon the addition of test biological sample, the number of sites displaying a detectable signal provides a quantitative indication of the expression level of the RQCD1 gene, the GIGYF1 gene or the GIGYF2 gene in the sample. The detection sites may be configured in any suitably detectable shape and are typically in the shape of a bar or dot spanning the width of a test strip.

III. Screening Methods:

Using the RQCD1 gene, the GIGYF1 gene or the GIGYF2 gene, polypeptides encoded by the genes or fragments thereof, or transcriptional regulatory region of the gene, it is possible to screen agents that alter the expression of the gene or the biological activity of a polypeptide encoded by the gene. Such agents may be used as pharmaceuticals for treating or preventing cancer, in particular, breast cancer. Thus, the present invention provides methods of screening for candidate agents for treating or preventing cancer using the RQCD1 gene, the GIGYF1 gene or the GIGYF2 gene, polypeptides encoded by the genes or fragments thereof, or transcriptional regulatory region of the gene.

An agent isolated by the screening method of the present invention is an agent that is expected to inhibit the expression of the RQCD1 gene, the GIGYF1 gene or the GIGYF2 gene, or the activity of the translation product of the gene, and thus, is a candidate for treating or preventing diseases attributed to, for example, cell proliferative diseases, such as cancer (in particular, breast cancer). Namely, the agents screened through the present methods are deemed to have a clinical benefit and can be further tested for its ability to prevent cancer cell growth in animal models or test subjects.

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, etc.) and natural compounds can be used in the screening methods of the present invention. Test agents useful in the screenings described herein can also be antibodies that specifically bind to a protein of interest or a partial peptide thereof that lacks the biological activity of the original proteins in vivo.

The test agent of the present invention can be also obtained using any of the numerous approaches in combinatorial library methods known in the art, including:

(1) biological libraries,

(2) spatially addressable parallel solid phase or solution phase libraries,

(3) synthetic library methods requiring deconvolution,

(4) the “one-bead one-compound” library method and

(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698, 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

Although the construction of test agent libraries is well known in the art, herein below, additional guidance in identifying test agents and construction libraries of such agents for the present screening methods are provided.

A. Molecular Modeling:

Construction of test agent libraries is facilitated by knowledge of the molecular structure of compounds known to have the properties sought, and/or the molecular structure of C12ORF48. One approach to preliminary screening of test agents suitable for further evaluation utilizes computer modeling of the interaction between the test agent and its target.

Computer modeling technology allows for the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles have been published on the subject of computer modeling of drugs interactive with specific proteins, examples of which include Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents suited to the treatment and/or prophylaxis of cancer and/or the prevention of post-operative recurrence of cancer, particularly breast cancer.

B. Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon EM. Curr Opin Biotechnol. 1995 Dec. 1; 6(6):624-31.; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

C. Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules (e.g., 10¹⁵ different molecules) can be used for screening.

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA finds use in preparing the test agent which is a candidate for treating or preventing cancer.

III-1. Protein Based Screening Methods

According to the present invention, the expression of the RQCD1 gene is crucial for the growth and/or survival of cancer cells, in particular breast cancer cells. Furthermore, the RQCD1 protein has been demonstrated to interact with the GIGYF1 protein and/or the GIGYF2 protein, and these three proteins were shown to be involved in Akt phosphorylation, which is well-known to closely linked to carcinogenesis. Accordingly, agents that suppress the function of the polypeptide encoded by the genes would be presumed to inhibit the growth and/or survival of cancer cells, and therefore find use in treating or preventing cancer. Thus, the present invention provides methods of screening a candidate agent for treating or preventing cancer, using the RQCD1 polypeptide, the GIGYF1 polypeptide or the GIGYF2 polypeptide. Further, the present invention also provides methods of screening a candidate agent for inhibiting the growth and/or survival of cancer cells, using the RQCD1 polypeptide, the GIGYF1 polypeptide or the GIGYF2 polypeptide. Furthermore, the present invention also provides methods of screening a candidate agent for inhibiting the Akt phosphorylation, specifically Ser 473 phosphorylation, using the using the RQCD1 polypeptide, the GIGYF1 polypeptide or the GIGYF2 polypeptide.

In addition to the RQCD1 polypeptide, the GIGYF1 polypeptide or the GIGYF2 polypeptide, fragments of the polypeptides may be used for the present screening, so long as it retains at least one biological activity of the natural occurring RQCD1 polypeptide, the GIGYF1 polypeptide or the GIGYF2 polypeptide.

The polypeptides or fragments thereof may be further linked to other substances, so long as the polypeptides and fragments retain at least one of their biological activity. Usable substances include: peptides, lipids, sugar and sugar chains, acetyl groups, natural and synthetic polymers, etc. These kinds of modifications may be performed to confer additional functions or to stabilize the polypeptide and fragments.

The polypeptides or fragments used for the present method may be obtained from nature as naturally occurring proteins via conventional purification methods or through chemical synthesis based on the selected amino acid sequence. For example, conventional peptide synthesis methods that can be adopted for the synthesis include:

1) Peptide Synthesis, Interscience, New York, 1966;

2) The Proteins, Vol. 2, Academic Press, New York, 1976;

3) Peptide Synthesis (in Japanese), Maruzen Co., 1975;

4) Basics and Experiment of Peptide Synthesis (in Japanese), Maruzen Co., 1985;

5) Development of Pharmaceuticals (second volume) (in Japanese), Vol. 14 (peptide synthesis), Hirokawa, 1991;

6) WO99/67288; and

7) Barany G. & Merrifield R. B., Peptides Vol. 2, “Solid Phase Peptide Synthesis”, Academic Press, New York, 1980, 100-118.

Alternatively, the proteins may be obtained through any known genetic engineering methods for producing polypeptides (e.g., Morrison J., J Bacteriology 1977, 132: 349-51; Clark-Curtiss & Curtiss, Methods in Enzymology (eds. Wu et al.) 1983, 101: 347-62). For example, first, a suitable vector including a polynucleotide encoding the objective protein in an expressible form (e.g., downstream of a regulatory sequence including a promoter) is prepared, transformed into a suitable host cell, and then the host cell is cultured to produce the protein. More specifically, a gene encoding the RQCD1 polypeptide, the GIGYF1 polypeptide or the GIGYF2 polypeptide are expressed in host (e.g., animal) cells and such by inserting the gene into a vector for expressing foreign genes, such as pSV2neo, pcDNA I, pcDNA3.1, pCAGGS, or pCD8. A promoter may be used for the expression. Any commonly used promoters may be employed including, for example, the SV40 early promoter (Rigby in Williamson (ed.), Genetic Engineering, vol. 3. Academic Press, London, 1982, 83-141), the EF-alpha promoter (Kim et al., Gene 1990, 91:217-23), the CAG promoter (Niwa et al., Gene 1991, 108:193), the RSV LTR promoter (Cullen, Methods in Enzymology 1987, 152:684-704), the SRalpha promoter (Takebe et al., Mol Cell Biol 1988, 8:466), the CMV immediate early promoter (Seed et al., Proc Natl Acad Sci USA 1987, 84:3365-9), the SV40 late promoter (Gheysen et al., J Mol Appl Genet. 1982, 1:385-94), the Adenovirus late promoter (Kaufman et al., Mol Cell Biol 1989, 9:946), the HSV TK promoter, and such. The introduction of the vector into host cells to express the RQCD1 gene, the GIGYF1 polypeptide or the GIGYF2 polypeptide can be performed according to any methods, for example, the electroporation method (Chu et al., Nucleic Acids Res 1987, 15:1311-26), the calcium phosphate method (Chen et al., Mol Cell Biol 1987, 7:2745-52), the DEAE dextran method (Lopata et al., Nucleic Acids Res 1984, 12:5707-17; Sussman et al., Mol Cell Biol 1985, 4:1641-3), the Lipofectin method (Derijard B, Cell 1994, 7:1025-37; Lamb et al., Nature Genetics 1993, 5:22-30; Rabindran et al., Science 1993, 259:230-4), and such.

The RQCD1 protein, the GIGYF1 protein or the GIGYF2 protein may also be produced in vitro adopting an in vitro translation system.

The RQCD1 polypeptide, the GIGYF1 polypeptide or the GIGYF2 polypeptide to be contacted with a test agent can be, for example, a purified polypeptide, a soluble protein, or a fusion protein fused with other polypeptides.

III-1-1. Identifying Agents that Bind to the Polypeptides

An agent that binds to a protein is likely to alter the expression of the gene coding for the protein or the biological activity of the protein. Thus, as an aspect, the present invention provides a method of screening a candidate agent for treating or preventing cancer, which includes steps of:

a) contacting a test agent with an RQCD1 polypeptide, a GIGYF1 polypeptide or a GIGYF2 polypeptide, or a fragment thereof;
b) detecting binding (or binding activity) between the polypeptide or fragment and the test agent; and
c) selecting the test agent that binds to the polypeptide as a candidate agent for treating or preventing cancer.

According to the present invention, the therapeutic effect of the test agent or compound on inhibiting the cell growth or a candidate agent or compound for treating or preventing RQCD1, GIGYF1 or GIGYF2 associating disease may be evaluated. Therefore, the present invention also provides a method of screening for a candidate agent or compound for inhibiting the cell growth or a candidate agent or compound for treating or preventing RQCD1, GIGYF1 or GIGYF2 associating disease, using the RQCD1, GIGYF1 or GIGYF2 polypeptide or fragments thereof including the steps as follows:

a) contacting a test agent with an RQCD1, a GIGYF1 or a GIGYF2 polypeptide or a fragment thereof;

b) detecting the binding (or binding activity) between the polypeptide or fragment and the test agent; and

c) correlating the binding of b) with the therapeutic effect of the test agent or compound.

In the context of the present invention, the therapeutic effect may be correlated with the binding level to RQCD1, GIGYF1 or GIGYF2 polypeptide or a functional fragment thereof. For example, when the test agent or compound binds to RQCD1, GIGYF1 or GIGYF2 polypeptide or a functional fragment thereof, the test agent or compound may identified or selected as the candidate agent or compound having the requisite therapeutic effect. Alternatively, when the test agent or compound does not bind to an RQCD1, GIGYF1 or GIGYF2 polypeptide or a functional fragment thereof, the test agent or compound may identified as the agent or compound having no significant therapeutic effect.

In the present invention, it is revealed that suppressing the expression of RQCD1, GIGYF1 or GIGYF2 reduces cancer cell growth. Thus, by screening for candidate compounds that binds to RQCD1, GIGYF1 or GIGYF2, candidate compounds that have the potential to treat or prevent cancers can be identified. Potential of these candidate compounds to treat or prevent cancers may be evaluated by second and/or further screening to identify therapeutic agent for cancers.

The binding of a test agent to the RQCD1 polypeptide, the GIGYF1 polypeptide or the GIGYF2 polypeptide may be, for example, detected by immunoprecipitation using an antibody against the polypeptide. Therefore, for the purpose for such detection, it is preferred that the RQCD1 polypeptide, the GIGYF1 polypeptide or the GIGYF2 polypeptide, or fragments thereof used for the screening contains an antibody recognition site. The antibody used for the screening may be one that recognizes an antigenic region (e.g., epitope) of the present RQCD1 polypeptide, the GIGYF1 polypeptide or the GIGYF2 polypeptide which preparation methods are well known in the art, and any method may be employed in the present invention to prepare such antibodies and equivalents thereof.

Alternatively, the RQCD1 polypeptide, the GIGYF1 polypeptide or the GIGYF2 polypeptide or a fragment thereof may be expressed as a fusion protein including at its N- or C-terminus a recognition site (epitope) of a monoclonal antibody, whose specificity has been revealed, to the N- or C-terminus of the polypeptide. A commercially available epitope-antibody system can be used (Experimental Medicine 1995, 13:85-90). Vectors which can express a fusion protein with, for example, beta-galactosidase, maltose binding protein, glutathione S-transferase, green florescence protein (GFP), and such by the use of its multiple cloning sites are commercially available and can be used for the present invention. Furthermore, fusion proteins containing much smaller epitopes to be detected by immunoprecipitation with an antibody against the epitopes are also known in the art (Experimental Medicine 1995, 13:85-90). Such epitopes, composed of several to a dozen amino acids so as not to change the property of the RQCD1 polypeptide, the GIGYF1 polypeptide or the GIGYF2 polypeptide, or fragments thereof, can also be used in the present invention. Examples include polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage), and such and monoclonal antibodies recognizing them can be used as the epitope-antibody system for screening proteins binding to the RQCD1 polypeptide (Experimental Medicine 13: 85-90 (1995)).

Glutathione S-transferase (GST) is also well-known as the counterpart of the fusion protein to be detected by immunoprecipitation. When GST is used as the protein to be fused with the RQCD1 polypeptide, the GIGYF1 polypeptide or the GIGYF2 polypeptide, or fragment thereof to form a fusion protein, the fusion protein can be detected either with an antibody against GST or a substance specifically binding to GST, i.e., such as glutathione (e.g., glutathione-Sepharose 4B).

In immunoprecipitation, an immune complex is formed by adding an antibody (recognizing the RQCD1 polypeptide, the GIGYF1 polypeptide or the GIGYF2 polypeptide, or a fragment thereof itself, or an epitope tagged to the polypeptide or fragment) to the reaction mixture of the RQCD1 polypeptide, the GIGYF1 polypeptide or the GIGYF2 polypeptide, and the test agent. If the test agent has the ability to bind the polypeptide, then the formed immune complex will consists of the RQCD1 polypeptide, the GIGYF1 polypeptide or the GIGYF2 polypeptide, the test agent, and the antibody. On the contrary, if the test agent is devoid of such ability, then the formed immune complex only consists of the RQCD1 polypeptide, the GIGYF1 polypeptide or the GIGYF2 polypeptide and the antibody. Therefore, the binding ability of a test agent to RQCD1 polypeptide, the GIGYF1 polypeptide or the GIGYF2 polypeptide can be examined by, for example, measuring the size of the formed immune complex. Any method for detecting the size of a substance can be used, including chromatography, electrophoresis, and such. For example, when mouse IgG antibody is used for the detection, Protein A or Protein G sepharose can be used for quantitating the formed immune complex.

For more details on immunoprecipitation see, for example, Harlow et al., Antibodies, Cold Spring Harbor Laboratory publications, New York, 1988, 511-52. SDS-PAGE is commonly used for analysis of immunoprecipitated proteins and the bound protein can be analyzed by the molecular weight of the protein using gels with an appropriate concentration. Detection may be achieved using conventional staining method, such as Coomassie staining or silver staining, or, for difficult to detect protections, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

Furthermore, the RQCD1 polypeptide, the GIGYF1 polypeptide or the GIGYF2 polypeptide, or a fragment thereof used for the screening of agents that bind to thereto may be bound to a carrier. Example of carriers that may be used for binding the polypeptides include insoluble polysaccharides, such as agarose, cellulose and dextran; and synthetic resins, such as polyacrylamide, polystyrene and silicon; preferably commercially available beads and plates (e.g., multi-well plates, biosensor chip, etc.) prepared from the above materials may be used. When using beads, they may be filled into a column. Alternatively, the use of magnetic beads is also known in the art, and enables to readily isolate polypeptides and agents bound on the beads via magnetism.

The binding of a polypeptide to a carrier may be conducted according to routine methods, such as chemical bonding and physical adsorption. Alternatively, a polypeptide may be bound to a carrier via antibodies specifically recognizing the protein. Moreover, binding of a polypeptide to a carrier can also be conducted by means of interacting molecules, such as the combination of avidin and biotin.

Screening using such carrier-bound RQCD1 polypeptide, the GIGYF1 polypeptide or the GIGYF2 polypeptide, or fragments thereof include, for example, contacting a test agent to the carrier-bound polypeptide, incubating the mixture, washing the carrier, and detecting and/or measuring the agent bound to the carrier. The binding may be carried out in buffer, for example, but are not limited to, phosphate buffer and Tris buffer, as long as the buffer does not inhibit the binding.

A screening method wherein such carrier-bound RQCD1 polypeptide, the GIGYF1 polypeptide or the GIGYF2 polypeptide, or fragments thereof and a composition (e.g., cell extracts, cell lysates, etc.) are used as the test agent, such method is generally called affinity chromatography. For example, the RQCD1 polypeptide, the GIGYF1 polypeptide or the GIGYF2 polypeptide may be immobilized on a carrier of an affinity column, and a test agent, containing a substance capable of binding to the polypeptides, is applied to the column. After loading the test agent, the column is washed, and then the substance bound to the polypeptide is eluted with an appropriate buffer.

A biosensor using the surface plasmon resonance phenomenon may be used as a mean for detecting or quantifying the bound agent in the present invention. When such a biosensor is used, the interaction between the RQCD1 polypeptide the GIGYF1 polypeptide or the GIGYF2 polypeptide, and a test agent can be observed real-time as a surface plasmon resonance signal, using only a minute amount of the polypeptide and without labeling (for example, BIAcore, Pharmacia). Therefore, it is possible to evaluate the binding between the polypeptide and test agent using a biosensor such as BIAcore.

Methods of screening for molecules that bind to a specific protein among synthetic chemical compounds, or molecules in natural substance banks or a random phage peptide display library by exposing the specific protein immobilized on a carrier to the molecules, and methods of high-throughput screening based on combinatorial chemistry techniques (Wrighton et al., Science 1996, 273:458-64; Verdine, Nature 1996, 384:11-3) to isolate not only proteins but chemical compounds are also well-known to those skilled in the art. These methods can also be used for screening agents (including agonist and antagonist) that bind to the RQCD1 protein, the GIGYF1 polypeptide or the GIGYF2 polypeptide, or fragments thereof.

When the test agent is a protein, for example, West-Western blotting analysis (Skolnik et al., Cell 1991, 65:83-90) can be used for the present method. Specifically, a protein binding to the RQCD1 polypeptide, the GIGYF1 polypeptide or the GIGYF2 polypeptide can be obtained by preparing first a cDNA library from cells, tissues, organs, or cultured cells (e.g., PC cell lines) expected to express at least one protein binding to the RQCD1 polypeptide, the GIGYF1 polypeptide or the GIGYF2 polypeptide using a phage vector (e.g., ZAP), expressing the proteins encoded by the vectors of the cDNA library on LB-agarose, fixing the expressed proteins on a filter, reacting the purified and labeled RQCD1 polypeptide, the GIGYF1 polypeptide or the GIGYF2 polypeptide with the above filter, and detecting the plaques expressing proteins to which the RQCD1 polypeptide, the GIGYF1 polypeptide or the GIGYF2 polypeptide has bound according to the label of the RQCD1 polypeptide, the GIGYF1 polypeptide or the GIGYF2 polypeptide.

Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, beta-glucosidase), fluorescent substances (e.g., fluorescein isothiocyanate (FITC), rhodamine) and biotin/avidin, may be used for the labeling of RQCD1 polypeptide, the GIGYF1 polypeptide or the GIGYF2 polypeptide in the present method. When the protein is labeled with radioisotope, the detection or measurement can be carried out by liquid scintillation. Alternatively, when the protein is labeled with an enzyme, it can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Moreover, the RQCD1 polypeptide, the GIGYF1 polypeptide or the GIGYF2 polypeptide bound to the protein can be detected or measured by utilizing an antibody that specifically binds to the RQCD1 polypeptide, the GIGYF1 polypeptide or the GIGYF2 polypeptide, or a peptide or polypeptide (for example, GST) that is fused to the RQCD1 polypeptide, the GIGYF1 polypeptide or the GIGYF2 polypeptide. In case of using an antibody in the present screening, the antibody is preferably labeled with one of the labeling substances mentioned above, and detected or measured based on the labeling substance. Alternatively, the antibody against the RQCD1 polypeptide, the GIGYF1 polypeptide or the GIGYF2 polypeptide may be used as a primary antibody to be detected with a secondary antibody that is labeled with a labeling substance. Furthermore, the antibody bound to the RQCD1 polypeptide, the GIGYF1 polypeptide or the GIGYF2 polypeptide in the present screening may be detected or measured using protein G or protein A column.

Alternatively, in another embodiment of the screening method of the present invention, two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton et al., Cell 1992, 68:597-612” and “Fields et al., Trends Genet. 1994, 10:286-92”). In two-hybrid system, RQCD1 polypeptide, the GIGYF1 polypeptide or the GIGYF2 polypeptide, or a fragment thereof is fused to the SRF-binding region or GAL4-binding region and expressed in yeast cells. A cDNA library is prepared from cells expected to express at least one protein binding to the RQCD1 polypeptide, the GIGYF1 polypeptide or the GIGYF2 polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the RQCD1 polypeptide, the GIGYF1 polypeptide or the GIGYF2 polypeptide is expressed in the yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used in addition to the HIS3 gene.

The agent isolated by this screening is a candidate for agonists or antagonists of the RQCD1 polypeptide, the GIGYF1 polypeptide or the GIGYF2 polypeptide. The term “agonist” refers to molecules that activate the function of the polypeptide by binding thereto. On the other hand, the term “antagonist” refers to molecules that inhibit the function of the polypeptide by binding thereto. Moreover, an agent isolated by this screening as an antagonist is a candidate that inhibits the in vivo interaction of the RQCD1 polypeptide, the GIGYF1 polypeptide or the GIGYF2 polypeptide with molecules (including nucleic acids (RNAs and DNAs) and proteins).

III-1-2. Identifying Agents by Detecting Biological Activity of the Polypeptides

The present invention also provides a method for screening a compound for treating or preventing cancer using the RQCD1 polypeptide, the GIGYF1 polypeptide or the GIGYF2 polypeptide, or fragments thereof including the steps as follows:

a) contacting a test agent or compound with an RQCD1 polypeptide, a GIGYF1 polypeptide or a GIGYF2 polypeptide, or a fragment thereof; and

b) detecting the biological activity of the polypeptide or fragment of the step (a).

c) selecting the test agent that reduces the biological activity of the polypeptide as compared to the biological activity in the absence of the test agent.

According to the present invention, the therapeutic effect of the test agent or compound on inhibiting the cell growth or a candidate agent or compound for treating or preventing RQCD1, GIGYF1 or GIGYF2 associating disease may be evaluated. Therefore, the present invention also provides a method of screening for a candidate agent or compound for inhibiting the cell growth or a candidate agent or compound for treating or preventing RQCD1, GIGYF1 or GIGYF2 associating disease, using the RQCD1, GIGYF1 or GIGYF2 polypeptide or fragments thereof including the steps as follows:

a) contacting a test agent or compound with an RQCD1, GIGYF1 or GIGYF2 polypeptide or a functional fragment thereof; and
b) detecting the biological activity of the polypeptide or fragment of step (a), and
c) correlating the biological activity of b) with the therapeutic effect of the test agent or compound.

In the present invention, the therapeutic effect may be correlated with the biological activity of an RQCD1, GIGYF1 or GIGYF2 polypeptide or a functional fragment thereof. For example, when the test agent or compound suppresses or inhibits the biological activity of an RQCD1, GIGYF1 or GIGYF2 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity RQCD1, GIGYF1 or GIGYF2 polypeptide or a functional fragment thereof as compared to a level detected in the absence of the test agent or compound, the test agent or compound may identified as the agent or compound having no significant therapeutic effect.

In the present invention, it is revealed that suppressing the expression of RQCD1, GIGYF1 or GIGYF2 reduces cancer cell growth. Thus, by screening for candidate compounds that suppresses the biological activity of the polypeptide, candidate compounds that have the potential to treat or prevent cancers can be identified. Potential of these candidate compounds to treat or prevent cancers may be evaluated by second and/or further screening to identify therapeutic agent for cancers. For example, when a compound binding to RQCD1, GIGYF1 or GIGYF2 protein inhibits described above activities of the cancer, it may be concluded that such compound has the RQCD1, GIGYF1 or GIGYF2 specific therapeutic effect.

Any polypeptide can be used for the screening so long it suppresses or reduces a biological activity of the RQCD1 polypeptide, the GIGYF1 polypeptide or the GIGYF2 polypeptide. In the context of the instant invention, the phrase “suppress or reduce a biological activity” encompasses at least 10% suppression of the biological activity of RQCD1, GIGYF1 and/or GIGYF2 in comparison with in absence of the compound, more preferably at least 25%, 50% or 75% suppression and most preferably at 90% suppression. Such suppression can serve an index in the present screening method.

According to the present invention, the RQCD1 polypeptide has been demonstrated to be required for the growth or viability of breast cancer cells. The biological α-tivities of the RQCD1 polypeptide that can be used as an index for the screening include such cell growth promoting activity of the human RQCD1 polypeptide.

When the biological activity to be detected in the present method is cell proliferation, it can be detected, for example, by preparing cells which express the RQCD1 polypeptide or a fragment thereof, culturing the cells in the presence of a test agent, and determining the speed of cell proliferation, measuring the cell cycle and such, as well as by detecting wound-healing activity, conducting Matrigel invasion assay and measuring the colony forming activity.

According to the present invention, the RQCD1 polypeptide interacts with the GIGYF1 protein and/or the GIGYF2 protein in vivo. Therefore, the RQCD1 polypeptide may be used together with GIGYF1 polypeptide and/or the GIGYF2 polypeptide. In this case, the method of the present invention may include the steps follows:

a) contacting a test agent or compound with an RQCD1 polypeptide or a fragment thereof and a GIGYF1 polypeptide and/or a GIGYF2 polypeptide or a fragment thereof; and

b) detecting the biological activity of the polypeptide or fragment of the step (a).

c) selecting the test agent that reduces the biological activity of the polypeptide as compared to the biological activity in the absence of the test agent.

According to the present invention, the RQCD1 polypeptide, the GIGYF1 polypeptide and the GIGYF2 polypeptide have Akt phosphorylation activity. Therefore, the Akt phosphorylation activity may be detected as a biological activity in the present screening method. When the biological activity to be detected is Akt phosphorylation, it can be detected, for example, by preparing cells which express the RQCD1 polypeptide, the GIGYF1 polypeptide or the GIGYF2 polypeptide, and determining the level of Akt phosphorylation, for example, using Western blotting with anti-phospho-Akt antibodies. The agents that reduce the level of Akt phosphorylation in cells expressed RQCD1 polypeptide, the GIGYF1 polypeptide or the GIGYF2 polypeptide as compared with that in untreated cells are selected as candidate agents. The RQCD1 gene may be co-transformed with the GIGYF1 gene and/or the GIGYF2 gene into a cell for preparing RQCD1-expressing cells.

Preferably, the phosphorylation level of Akt may be detected at the 473 serine residue. The example of the amino acid sequence of the Akt polypeptide is shown in SEQ ID NO: 40 (GeneBank Accession No. NP_{—}001014431), which is encoded by nucleotide sequence of SEQ ID NO: 39 (GeneBank Accession No. NM_{—}001014431.1). The phosphorylation at the 473 Ser can be detected, for example, by Western blotting using anti-phospho-Akt (Ser 473).

In addition to screening methods using cells expressing the RQCD1 polypeptide, the GIGYF1 polypeptide or the GIGYF2 polypeptide, isolated the RQCD1 polypeptide, the GIGYF1 polypeptide or the GIGYF2 polypeptide may be used for the present screening methods. In this case, isolated Akt may be provided in reaction systems with appropriate phosphate donor (e.g., ATP). Alternatively, Akt may be captured by contacting Akt with a carrier having an anti-Akt antibody. When the labeled phosphate donor is used, the phosphorylation level of the Akt can be detected by tracing the label. For example, when radio-labeled ATP (e.g., ³²P-ATP) is used as a phosphate donor, radio activity incorporated into Akt correlates with the phosphorylation level of the Akt.

According to the present invention, the RQCD1 polypeptide, the GIGYF1 polypeptide and the GIGYF2 polypeptide is interacted with each other in vivo. Therefore, a combination among the RQCD1 polypeptide, the GIGYF1 polypeptide and the GIGYF2 polypeptide may be used for the present screening methods.

The agent isolated by the present screening method is a candidate for an antagonist of the RQCD1 polypeptide, and thus, is a candidate that inhibits the in vivo interaction of the polypeptide with molecules (including nucleic acids (RNAs and DNAs) and proteins).

III-1-3. Identifying Agents by Detecting the Binding Activity Among the RQCD1 Polypeptide and the GIGYF1 Polypeptide and/or the GIGYF2 Polypeptide

According to the present invention, the RQCD1 polypeptide interacts with the GIGYF1 polypeptide and/or GIGYF2 polypeptide in cancer cells, particularly breast cancer cells, and the interaction among theses polypeptides is considered to be important for Akt phosphorylation and cancer cell growth. Therefore, agents that inhibit the interaction among the RQCD1 polypeptide, the GIGYF1 polypeptide and GIGYF2 polypeptide are expected to be useful for inhibiting Akt phosphorylation and cancer cell growth and/or survival, thus useful for treating or preventing cancer. Thus, the present invention provides methods of screening for candidate agents for treating or preventing cancer based on the binding activity among the RQCD1 polypeptide, the GIGYF1 polypeptide and GIGYF2 polypeptide. The present screening methods are also useful for screening for a candidate agent for inhibiting cancer cell growth and/or survival, and Akt phosphorylation. The present screening methods include the steps of:

(1) contacting a GIGYF1 polypeptide and/or a GIGYF2 polypeptide or functional equivalent thereof with an RQCD1 polypeptide or functional equivalent thereof in the presence of a test agent;

(2) detecting the binding between the polypeptides of the step (1); and

(3) selecting the test agent that inhibits the binding between the polypeptides.

According to the present invention, the therapeutic effect of the test agent or compound on inhibiting the cell growth or a candidate agent or compound for treating or preventing RQCD1, GIGYF1 or GIGYF2 associating disease may be evaluated. Therefore, the present invention also provides a method of screening for a candidate agent or compound for inhibiting the cell growth or a candidate agent or compound for treating or preventing RQCD1, GIGYF1 or GIGYF2 associating disease, using the RQCD1, GIGYF1 or GIGYF2 polypeptide or fragments thereof including the steps as follows:

a) contacting a GIGYF1 polypeptide and/or a GIGYF2 polypeptide or functional equivalent thereof with an RQCD1 polypeptide or functional equivalent thereof in the presence of a test agent;
b) detecting the binding between the polypeptides of the step (a); and
c) correlating the binding of b) with the therapeutic effect of the test agent or compound.

In the present invention, the therapeutic effect may be correlated with the binding activity among the RQCD1 polypeptide, the GIGYF1 polypeptide and GIGYF2 polypeptide or a functional fragment thereof. For example, when the test agent or compound suppresses or inhibits binding activity among the RQCD1 polypeptide, the GIGYF1 polypeptide and GIGYF2 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit binding activity among the RQCD1 polypeptide, the GIGYF1 polypeptide and GIGYF2 polypeptide or a functional fragment thereof as compared to a level detected in the absence of the test agent or compound, the test agent or compound may identified as the agent or compound having no significant therapeutic effect.

In the present invention, it is revealed that suppressing the binding activity among the RQCD1 polypeptide, the GIGYF1 polypeptide and GIGYF2 polypeptide or a functional fragment thereof reduces cancer cell growth. Thus, by screening for candidate compounds that suppresses the binding activity, candidate compounds that have the potential to treat or prevent cancers can be identified. The potential of these candidate compounds to treat or prevent cancers may be evaluated by second and/or further screening to identify therapeutic agent for cancers.

As a method of screening for agents that inhibit the binding between the RQCD1 polypeptide and the GIGYF1 polypeptide and/or the GIGYF2 polypeptide, many methods well known by one skilled in the art can be used. For example, screening can be carried out as an in vitro assay system, such as a cellular system. More specifically, first, either the RQCD1 polypeptide or the GIGYF1 polypeptide and/or the GIGYF2 polypeptide is bound to a support, and the other protein is added together with a test agent thereto. Next, the mixture is incubated, washed and the other protein bound to the support is detected and/or measured.

Examples of supports that may be used for binding proteins include, for example, insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads is also known in the art, and enables one to readily isolate proteins bound on the beads via magnetism.

The binding of a protein to a support may be conducted according to routine methods, such as chemical bonding and physical adsorption, for example. Alternatively, a protein may be bound to a support via antibodies that specifically recognize the protein. Moreover, binding of a protein to a support can be also conducted by means of avidin and biotin.

The binding between proteins is preferably carried out in buffer, examples of which include, but are not limited to, phosphate buffer and Tris buffer. However, the selected buffer must not inhibit binding between the proteins.

In the context of the present invention, a biosensor using the surface plasmon resonance phenomenon may be used as a mean for detecting or quantifying the bound protein. When such a biosensor is used, the interaction between the proteins can be observed in real-time as a surface plasmon resonance signal, using only a minute amount of polypeptide and without labeling (for example, BIAcore, Pharmacia). Therefore, it is possible to evaluate binding between the RQCD1 polypeptide and the GIGYF1 polypeptide and/or the GIGYF2 polypeptide using a biosensor such as BIAcore.

Alternatively, either the RQCD1 polypeptide or the GIGYF1 polypeptide and/or the GIGYF2 polypeptide may be labeled, and the label of the bound protein may be used to detect or measure the bound protein. Specifically, after pre-labeling one of the proteins, the labeled protein is contacted with the other protein in the presence of a test agent, and then bound proteins are detected or measured according to the label after washing.

Labeling substances including but not limited to radioisotope (e.g., ³H, ¹⁴C, ³²P, ³³P, ³⁵S, ¹²⁵I, ¹³¹I), enzymes (e.g., alkaline phosphatase, horseradish peroxidase, beta-galactosidase, beta-glucosidase), fluorescent substances (e.g., fluorescein isothiocyanate (FITC), rhodamine) and biotin/avidin may be used for the labeling of a protein in the present method. When the protein is labeled with a radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding of the RQCD1 polypeptide and the GIGYF1 polypeptide and/or the GIGYF2 polypeptide can be also detected or measured using antibodies to the RQCD1 polypeptide, the GIGYF1 polypeptide or the GIGYF2 polypeptide. For example, after contacting the RQCD1 polypeptide immobilized on a support with a test agent and the GIGYF1 polypeptide and/or the GIGYF2 polypeptide, the mixture is incubated and washed, and detection or measurement can be conducted using an antibody against the GIGYF1 polypeptide and/or the GIGYF2 polypeptide. Alternatively, the GIGYF1 polypeptide and/or the GIGYF2 polypeptide may be immobilized on a support, and an antibody against the RQCD1 polypeptide may be used as the antibody.

When using an antibody in the present screening, the antibody is preferably labeled with one of the labeling substances mentioned above, and detected or measured based on the labeling substance. Alternatively, an antibody against the RQCD1 polypeptide, the GIGYF1 polypeptide or the GIGYF2 polypeptide may be used as a primary antibody to be detected with a secondary antibody that is labeled with a labeling substance. Furthermore, an antibody bound to the protein in the screening of the present invention may be detected or measured using protein G or protein A column.

The polypeptides to be used in the present screening methods may be recombinantly produced using standard procedures. For example, a gene encoding a polypeptide of interest may be expressed in animal cells by inserting the gene into an expression vector for foreign genes, such as pSV2neo, pcDNA I, pcDNA3.1, pCAGGS and pCD8. The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466-72 (1988)), the CMV immediate early promoter (Seed and Aruffo, Proc Natl Acad Sci USA 84: 3365-9 (1987)), the SV40 late promoter (Gheysen and Fiers, J Mol Appl Genet. 1: 385-94 (1982)), the Adenovirus late promoter (Kaufman et al., Mol Cell Biol 9: 946-58 (1989)), the HSV TK promoter and so on. The introduction of the gene into animal cells to express a foreign gene can be performed according to any conventional method, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B, Cell 76: 1025-37 (1994); Lamb et al., Nature Genetics 5: 22-30 (1993): Rabindran et al., Science 259: 230-4 (1993)), and so on. The polypeptides may be expressed as a fusion protein including a recognition site (epitope) of a monoclonal antibody by introducing the epitope of the monoclonal antibody, whose specificity has been revealed, to the N- or C-terminus of the polypeptide. Alternatively, a commercially available epitope-antibody system may be used (Experimental Medicine 13: 85-90 (1995)). Vectors which are capable of expressing a fusion protein with, for example, beta-galactosidase, maltose binding protein, glutathione S-transferase, green florescence protein (GFP), and so on, by the use of its multiple cloning sites are commercially available.

A fusion protein, prepared by introducing only small epitopes composed of several to a dozen amino acids so as not to change the property of the original polypeptide by the fusion, is also provided herein. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and antibodies recognizing them may be used as the epitope-antibody system for detecting the binding activity between the polypeptides (Experimental Medicine 13: 85-90 (1995)).

Antibodies to be used in the present screening methods can be prepared using techniques well known in the art. Antigens to prepared antibodies may be derived from any animal species, but preferably is derived from a mammal such as a human, mouse, rabbit, or rat, more preferably from a human. The polypeptide used as the antigen can be recombinantly produced or isolated from natural sources. The polypeptides to be used as an immunization antigen may be a complete protein or a partial peptide derived from the complete protein.

Any mammalian animal may be immunized with the antigen; however, the compatibility with parental cells used for cell fusion is preferably taken into account. In general, animals of the order Rodentia, Lagomorpha or Primate are used. Animals of the Rodentia order include, for example, mice, rats and hamsters. Animals of Lagomorpha order include, for example, hares, pikas, and rabbits. Animals of Primate order include, for example, monkeys of Catarrhini (old world monkey) such as Macaca fascicularis, rhesus monkeys, sacred baboons and chimpanzees.

Methods for immunizing animals with antigens are well known in the art. Intraperitoneal injection or subcutaneous injection of antigens is a standard method for immunizing mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion, and then administered to mammalian animals. Preferably, it is followed by several administrations of the antigen mixed with an appropriately amount of Freund's incomplete adjuvant every 4 to 21 days. An appropriate carrier may also be used for immunization. After immunization as above, the serum is examined by a standard method for an increase in the amount of desired antibodies.

Polyclonal antibodies may be prepared by collecting blood from the immunized mammal examined for the increase of desired antibodies in the serum, and by separating serum from the blood by any conventional method. Polyclonal antibodies include serum containing the polyclonal antibodies, as well as the fraction containing the polyclonal antibodies isolated from the serum. Immunoglobulin G or M can be prepared from a fraction which recognizes only the objective polypeptide using, for example, an affinity column coupled with the polypeptide, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al., (Galfre and Milstein, Methods Enzymol 73: 3-46 (1981)).

Resulting hybridomas obtained by the cell fusion may be selected by cultivating them in a standard selection medium, such as HAT medium (hypoxanthine, aminopterin, and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes, such as those infected by the EB virus, may be immunized with an antigen, cells expressing such antigen, or their lysates in vitro. Then, the immunized lymphocytes are fused with human-derived myeloma cells that are capable of indefinitely dividing, such as U266, to yield a hybridoma producing a desired human antibody that is able to bind to the antigen (Unexamined Published Japanese Patent Application No. (JP-A) Sho 63-17688).

The obtained hybridomas may be subsequently transplanted into the abdominal cavity of a mouse and the ascites may be extracted. The obtained monoclonal antibodies can be purified by, for example, ammonium sulfate precipitation, a protein A or protein G column, DEAE ion exchange chromatography, or an affinity column carrying an objective antigen.

Antibodies against the RQCD1 polypeptide, the GIGYF1 polypeptide or the GIGYF2 polypeptide can be used not only in the present screening method, but also for the detection of the polypeptides as cancer markers in biological samples as described in “I. Diagnosing cancer”. They may further serve as candidates for agonists and antagonists of the polypeptides of interest. In addition, such antibodies, serving as candidates for antagonists, can be applied to the antibody treatment for diseases related to the RQCD1 polypeptide, the GIGYF1 polypeptide or the GIGYF2 polypeptide, including breast cancer as described infra.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. Such recombinant antibody can also be used in the context of the present screening.

Furthermore, antibodies used in the screening and so on may be fragments of antibodies or modified antibodies, so long as they retain the original binding activity. For instance, the antibody fragment may be an Fab, F(ab′)₂, Fv, or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding an antibody fragment may be constructed, inserted into an expression vector, and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). Modified antibodies can be obtained through chemically modification of an antibody. These modification methods are conventional in the field.

Antibodies obtained as above may be purified to homogeneity. For example, the separation and purification of the antibody can be performed according to separation and purification methods used for general proteins. For example, the antibody may be separated and isolated by appropriately selected and combined column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)); however, the present invention is not limited thereto. A protein A column and protein G column can be used as the affinity column. Exemplary protein A columns to be used include, for example, Hyper D, POROS, and Sepharose F. F. (Pharmacia).

Exemplary chromatography, with the exception of affinity, includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

Alternatively, a two-hybrid system utilizing cells may be used for detecting or measuring the binding activity among the polypeptides (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, for example, the RQCD1 polypeptide are fused to the SRF-binding region or GAL4-binding region and expressed in yeast cells. The GIGYF1 polypeptide or the GIGYF2 polypeptide are fused to the VP16 or GAL4 transcriptional activation region and also expressed in the yeast cells in the existence of a test agent. Alternatively, the GIGYF1 polypeptide or the GIGYF2 polypeptide may be fused to the SRF-binding region or GAL4-binding region, and the RQCD1 polypeptide may be fused to the VP16 or GAL4 transcriptional activation region. The binding of the two polypeptides activates a reporter gene, making positive clones detectable. As a reporter gene, for example, Ade2 gene, lacZ gene, CAT gene, luciferase gene and such can be used besides HIS3 gene.

III-2. Nucleotide Based Screening Methods

III-2-1. Screening Method Using RQCD1 Gene, GIGYF1 Gene or GIGYF2 Gene

As discussed in detail above, by controlling the expression level of the RQCD1 gene, the GIGYF1 gene or the GIGYF2 gene, one can control the onset and progression of cancer. Thus, agents that may be used in the treatment or prevention of cancers can be identified through screenings that use the expression levels of the RQCD1 gene, the GIGYF1 gene or the GIGYF2 gene as indices. In the context of the present invention, such screening may include, for example, the following steps:

a) contacting a test agent or compound with a cell expressing an RQCD1 gene, a GIGYF1 gene or a GIGYF2 gene;
b) detecting the expression level of the RQCD1 gene, the GIGYF1 gene or the GIGYF2 gene;
c) comparing the expression level with the expression level detected in the absence of the agent; and
d) selecting the agent that reduces the expression level as a candidate agent for treating or preventing cancer.

According to the present invention, the therapeutic effect of the test agent or compound on inhibiting the cell growth or a candidate agent or compound for treating or preventing RQCD1, GIGYF1 or GIGYF2 associating disease may be evaluated. Therefore, the present invention also provides a method for screening a candidate agent or compound that suppresses the proliferation of cancer cells, and a method for screening a candidate agent or compound for treating or preventing RQCD1, GIGYF1 or GIGYF2 associating disease.

In the context of the present invention, such screening may include, for example, the following steps:

a) contacting a test agent or compound with a cell expressing an RQCD1, GIGYF1 or GIGYF2 gene;

b) detecting the expression level of the RQCD1, GIGYF1 or GIGYF2 gene; and

c) correlating the expression level of b) with the therapeutic effect of the test agent or compound.

In the context of the present invention, the therapeutic effect may be correlated with the expression level of the RQCD1, GIGYF1 or GIGYF2 gene. For example, when the test agent or compound reduces the expression level of the RQCD1, GIGYF1 or GIGYF2 gene as compared to a level detected in the absence of the test agent or compound, the test agent or compound may identified or selected as the candidate agent or compound having the therapeutic effect. Alternatively, when the test agent or compound does not reduce the expression level of the RQCD1, GIGYF1 or GIGYF2 gene as compared to a level detected in the absence of the test agent or compound, the test agent or compound may identified as the agent or compound having no significant therapeutic effect.

Herein, it was revealed that suppressing the expression of RQCD1, GIGYF1 or GIGYF2 reduces cancer cell growth. Thus, by screening for candidate compounds that reduces the expression level of RQCD1, GIGYF1 or GIGYF2, candidate compounds that have the potential to treat or prevent cancers can be identified. Potential of these candidate compounds to treat or prevent cancers may be evaluated by second and/or further screening to identify therapeutic agent for cancers.

An agent that inhibits the expression of the RQCD1 gene, the GIGYF1 gene or the GIGYF2 gene or the activity of its gene product can be identified by contacting a cell expressing the RQCD1 gene, the GIGYF1 gene or the GIGYF2 gene with a test agent and then determining the expression level of the RQCD1 gene, the GIGYF1 gene or the GIGYF2 gene. Naturally, the identification may also be performed using a population of cells that express the gene in place of a single cell. A decreased expression level detected in the presence of an agent as compared to the expression level in the absence of the agent indicates the agent as being an inhibitor of the RQCD1 gene, the GIGYF1 gene or the GIGYF2 gene, suggesting the possibility that the agent is useful for inhibiting cancer, thus a candidate agent to be used for the treatment or prevention of cancer.

The expression level of a gene can be estimated by methods well known to one skilled in the art. The expression level of the RQCD1 gene, the GIGYF1 gene or the GIGYF2 gene can be, for example, determined following the method described above under the item of ‘I-1. Method for diagnosing cancer or a predisposition for developing cancer’.

The cell or the cell population used for such identification may be any cell or any population of cells so long as it expresses the RQCD1 gene, the GIGYF1 gene or the GIGYF2 gene. For example, the cell or population may be or contain a breast epithelial cell derived from a tissue. Alternatively, the cell or population may be or contain an immortalized cell derived from a carcinoma cell, including breast cancer cell. Cells expressing the RQCD1 gene, the GIGYF1 gene or the GIGYF2 gene include, for example, cell lines established from cancers (e.g., breast cancer cell lines such as HCC-1937, BT-549, MCF-7, BSY-1, MDA-MB-435S, SKBR-3, T-47D, MDA-MB-231, YMB-1 etc.). Furthermore, the cell or population may be or contain a cell which has been transfected with the RQCD1 gene, the GIGYF1 gene or the GIGYF2 gene.

The present method allows screening of various agents mentioned above and is particularly suited for screening functional nucleic acid molecules including antisense RNA, siRNA, and such.

III 2-2. Screening Method Using Transcriptional Regulatory Region of RQCD1 Gene, GIGYF1 Gene or GIGYF2 Gene

According to another aspect, the present invention provides a method which includes the following steps of:

a) contacting a test agent or compound with a cell into which a vector, including a transcriptional regulatory region of an RQCD1 gene, a GIGYF1 gene or a GIGYF2 gene and a reporter gene that is expressed under the control of the transcriptional regulatory region, has been introduced;

b) detecting the expression or activity of said reporter gene;

c) comparing the expression level or activity with the expression level or activity detected in the absence of the agent; and

d) selecting the agent that reduces the expression or activity of said reporter gene as a candidate agent for treating or preventing cancer.

According to the present invention, the therapeutic effect of the test agent or compound on inhibiting the cell growth or a candidate agent or compound for treating or preventing RQCD1, GIGYF1 or GIGYF2 associating disease may be evaluated. Therefore, the present invention also provides a method for screening a candidate agent or compound that suppresses the proliferation of cancer cells, and a method for screening a candidate agent or compound for treating or preventing RQCD1, GIGYF1 or GIGYF2 associating disease.

According to another aspect, the present invention provides a method which includes the following steps of:

a) contacting a test agent or compound with a cell into which a vector, composed of a transcriptional regulatory region of an RQCD1, GIGYF1 or GIGYF2 gene and a reporter gene that is expressed under the control of the transcriptional regulatory region, has been introduced;

b) detecting the expression or activity of said reporter gene; and

c) correlating the expression level of b) with the therapeutic effect of the test agent or compound.

In the present invention, the therapeutic effect may be correlated with the expression or activity of said reporter gene. For example, when the test agent or compound reduces the expression or activity of said reporter gene as compared to a level detected in the absence of the test agent or compound, the test agent or compound may identified or selected as the candidate agent or compound having the therapeutic effect. Alternatively, when the test agent or compound does not reduce the expression or activity of said reporter gene as compared to a level detected in the absence of the test agent or compound, the test agent or compound may identified as the agent or compound having no significant therapeutic effect.

Herein, it was revealed that suppressing the expression of RQCD1, GIGYF1 or GIGYF2 reduces cell growth. Thus, by screening for candidate compounds that reduces the expression or activity of said reporter gene, candidate compounds that have the potential to treat or prevent cancers can be identified. Potential of these candidate compounds to treat or prevent cancers may be evaluated by second and/or further screening to identify therapeutic agent for cancers.

Suitable reporter genes and host cells are well known in the art. The reporter construct required for the screening can be prepared using the transcriptional regulatory region of the RQCD1 gene, the GIGYF1 gene or the GIGYF2 gene, which can be obtained as a nucleotide segment containing the transcriptional regulatory region from a genome library based on the nucleotide sequence information of the gene.

The transcriptional regulatory region may be, for example, the promoter sequence of the RQCD1 gene, the GIGYF1 gene or the GIGYF2 gene. The reporter construct required for the screening can be prepared by connecting reporter gene sequence to the transcriptional regulatory region of RQCD1, GIGYF1 or GIGYF2 gene. The transcriptional regulatory region of RQCD1, GIGYF1 or GIGYF2 gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

When a cell(s) transfected with a reporter gene that is operably linked to the regulatory sequence (e.g., promoter sequence) of the RQCD1 gene, the GIGYF1 gene or the GIGYF2 gene is used, an agent can be identified as inhibiting or enhancing the expression of the RQCD1 gene, the GIGYF1 gene or the GIGYF2 gene through detecting the expression level of the reporter gene product.

Illustrative reporter genes include, but are not limited to, luciferase, green florescence protein (GFP), Discosoma sp. Red Fluorescent Protein (DsRed), Chrolamphenicol Acetyltransferase (CAT), lacZ and beta-glucuronidase (GUS), and host cell is COS 7, HEK293, HeLa, Ade2 gene, HIS3 gene, and others well-known in the art. Methods for detection of the expression of these genes are well known in the art.

A vector containing a reporter construct may be infected to host cells and the expression or activity of the reporter gene is detected by method well known in the art (e.g., using luminometer, absorption spectrometer, flow cytometer and so on). In the context of the instant invention, the phrase “reduces the expression or activity” encompasses at least 10% reduction of the expression or activity of the reporter gene in comparison with in absence of the compound, more preferably at least 25%, 50% or 75% reduction and most preferably at 95% reduction.

III-3. Selecting Therapeutic Agents that are Appropriate for a Particular Individual

Differences in the genetic makeup of individuals can result in differences in their relative abilities to metabolize various drugs. An agent that is metabolized in a subject to act as an anti-tumor agent can manifest itself by inducing a change in a gene expression pattern in the subject's cells from that characteristic of a cancerous state to a gene expression pattern characteristic of a non cancerous state. Accordingly, the RQCD1 gene, the GIGYF1 gene or the GIGYF2 gene differentially expressed between cancerous and non-cancerous cells disclosed herein allow for a putative therapeutic or prophylactic inhibitor of cancer to be tested in a test cell population from a selected subject in order to determine if the agent is a suitable inhibitor of cancer in the subject.

To identify an inhibitor of cancer that is appropriate for a specific subject, a test cell population from the subject is exposed to a candidate therapeutic agent, and the expression of RQCD1 gene, the GIGYF1 gene or the GIGYF2 gene is determined.

In the context of the method of the present invention, test cell populations contain cancer cells expressing the RQCD1 gene, the GIGYF1 gene or the GIGYF2 gene. Preferably, the test cell is a breast epithelial cell.

Specifically, a test cell population may be incubated in the presence of a candidate therapeutic agent and the expression of the RQCD1 gene, the GIGYF1 gene or the GIGYF2 gene in the test cell population may be measured and compared to one or more reference profiles, e.g., a cancerous reference expression profile or a non-cancerous reference expression profile.

A decrease in the expression of the RQCD1 gene, the GIGYF1 gene or the GIGYF2 gene in a test cell population relative to a reference cell population containing cancer indicates that the agent has therapeutic potential. Alternatively, a similarity in the expression of the RQCD1 gene, the GIGYF1 gene or the GIGYF2 gene in a test cell population relative to a reference cell population not containing cancer indicates that the agent has therapeutic potential.

IV. Pharmaceutical Compositions for Treating or Preventing Cancer:

The agents screened by any of the screening methods of the present invention, antisense nucleic acids and double-stranded molecules (e.g., siRNA) of the RQCD1 gene, the GIGYF1 gene or the GIGYF2 gene, and antibodies against the RQCD1 polypeptide, the GIGYF1 polypeptide or the GIGYF2 polypeptide inhibit or suppress the expression of the RQCD1 gene, the GIGYF1 gene or the GIGYF2 gene, or the biological activity of the RQCD1 polypeptide, the GIGYF1 polypeptide or the GIGYF2 polypeptide and inhibit or disrupts cancer cell cycle regulation and cancer cell proliferation. Thus, the present invention provides compositions for treating or preventing cancer, which compositions include agents screened by any of the screening methods of the present invention, antisense nucleic acids and double-stranded molecules of the RQCD1 gene, the GIGYF1 gene or the GIGYF2 gene, or antibodies against the RQCD1 polypeptide, the GIGYF1 polypeptide or the GIGYF2 polypeptide. The present compositions can be used for treating or preventing cancer, in particular, cancer such as breast cancer, lung cancer and esophageal cancer, in more particular breast cancer. Further, the present compositions may be used for inhibiting cancer cell proliferation or Akt phosphorylation.

The compositions may be used as pharmaceuticals for humans and other mammals, such as mice, rats, guinea-pigs, rabbits, cats, dogs, sheep, pigs, cattle, monkeys, baboons, and chimpanzees.

In the context of the present invention, suitable pharmaceutical formulations for the active ingredients of the present invention detailed below (including screened agents, antisense nucleic acids, double-stranded molecules, antibodies, etc.) include those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include capsules, microcapsules, cachets and tablets, each containing a predetermined amount of active ingredient. Suitable formulations also include powders, elixirs, granules, solutions, suspensions and emulsions. The active ingredient is optionally administered as a bolus electuary or paste. Alternatively, according to needs, the pharmaceutical composition may be administered non-orally, in the form of injections of sterile solutions or suspensions with water or any other pharmaceutically acceptable liquid. For example, the active ingredients of the present invention can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. A tablet may be made by compression or molding, optionally with one or more formulational ingredients. Compressed tablets may be prepared by compressing in a suitable machine the active ingredients in a free-flowing form such as powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art. The tablets may optionally be formulated so as to provide slow or controlled release of the active ingredient in vivo. A package of tablets may contain one tablet to be taken on each of the month.

Furthermore, when the unit-dosage form is a capsule, a liquid carrier, such as oil, can be further included in addition to the above ingredients.

Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Moreover, sterile composites for injection can be formulated following normal drug implementations using vehicles, such as distilled water, suitable for injection. Physiological saline, glucose, and other isotonic liquids, including adjuvants, such as D-sorbitol, D-mannose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80 ™) and HCO-50.

Sesame oil or soy-bean oil can be used as an oleaginous liquid, which may be used in conjunction with benzyl benzoate or benzyl alcohol as a solubilizer, and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles including the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also including one or more dispersing agents, solubilizing agents or suspending agents.

For administration by inhalation the compositions are conveniently delivered from an insufflator, nebulizer, pressurized packs or other convenient means of delivering an aerosol spray. Pressurized packs may include a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other formulations include implantable devices and adhesive patches that release a therapeutic agent.

When desired, the above-described formulations, adapted to give sustained release of the active ingredient, may be employed. The pharmaceutical compositions may also contain other active ingredients such as antimicrobial agents, immunosuppressants or preservatives.

It should be understood that, in addition to the ingredients particularly mentioned above, the formulations of this invention may include other agents conventional in the art having regard to the type of formulation in question; for example, those suitable for oral administration may include flavoring agents.

Preferred unit dosage formulations are those containing an effective dose, as recited under the item of ‘V. Method for treating or preventing cancer’ (infra), of each of the active ingredients of the present invention or an appropriate fraction thereof.

IV-1. Pharmaceutical Compositions Containing Screened Agents

The present invention provides compositions for treating or preventing cancers including any of the agents selected by the above-described screening methods of the present invention.

An agent screened by the method of the present invention can be directly administered or can be formulated into a dosage form according to any conventional pharmaceutical preparation method detailed above.

IV-2. Pharmaceutical Compositions Including Double-Stranded Molecules

Double-stranded molecules (e.g., siRNA) against the RQCD1 gene, the GIGYF1 gene or the GIGYF2 gene can be used to reduce the expression level of the genes. Herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)) as described in “Definitions”. In the context of the present invention, double-stranded molecules include a sense nucleic acid sequence and an anti-sense nucleic acid sequence against the RQCD1 gene, the GIGYF1 gene or the GIGYF2 gene. The double-stranded molecule is constructed so that it both includes a portion of the sense and complementary antisense sequences of the target gene (i.e., the RQCD1 gene, the GIGYF1 gene or the GIGYF2 gene), and may also be a single construct taking a hairpin structure, wherein the sense and antisense strands are linked via a single-strand.

A double-stranded molecule hybridizes to target mRNA, i.e., associates with the normally single-stranded mRNA transcript and thereby interfering with translation of the mRNA, which finally decreases or inhibits production (expression) of the polypeptide encoded by the gene. Thus, a double-stranded molecule of the invention can be defined by its ability to specifically hybridize to the mRNA of the RQCD1 gene, the GIGYF1 gene or the GIGYF2 gene under stringent conditions. Herein, the portion of the double-stranded molecule that hybridizes with the target mRNA is referred to as “target sequence” or “target nucleic acid” or “target nucleotide”.

In the context of the present invention, the target sequence of a double-stranded molecule is preferably less than 500, 200, 100, 50, or 25 base pairs in length. More preferably, the target sequence of a double stranded molecule is 19-25 base pairs in length. Exemplary target nucleic acid sequences of double-stranded molecules against the RQCD1 gene include the nucleotide sequences of SEQ ID NO: 8, 9 30 or 31. Exemplary target nucleic acid sequences of double-stranded molecules against the GIGYF1 gene include the nucleotide sequences of SEQ ID NO: 32. Exemplary target nucleic acid sequences of double-stranded molecules against the GIGYF2 gene include the nucleotide sequences of SEQ ID NO: 33. The nucleotide “t” in the sequence should be replaced with “u” in RNA or derivatives thereof. Accordingly, for example, the present pharmaceutical composition may include a double-stranded RNA molecule (siRNA) including the nucleotide sequence 5′-AAGAUCUAUCAGUGGAUCAAU-3′ (for SEQ ID NO: 8), 5′-AAGAUCUUGUUAGAUGACACU-3′ (for SEQ ID NO: 9), 5′-GAUCUAUCAGUGGAUCAAU-3′ (for SEQ ID NO: 30), 5′-GAUCUUGUUAGAUGACACU-3′ (for SEQ ID NO: 31), 5′-CCUUCCGAAGGGCUAGAGG-3′ (for SEQ ID NO:32) or 5′-CAAGAUACCUUCAGACCUU-3′ (for SEQ ID NO:33) as the sense strand.

In order to enhance the inhibition activity of the double-stranded molecule, 3′ overhangs can be added to the 3′ end of the target sequence in the sense and/or antisense strand. The number of nucleotides to be added is at least 2, generally 2 to 10, preferably 2 to 5. The added nucleotides form a single strand at the 3′ end of the sense and/or antisense strand of the double-stranded molecule. The nucleotides to be added is preferably “u” or “t”, but are not limited to.

A loop sequence consisting of an arbitrary nucleotide sequence can be located between the sense and antisense strands in order to form a hairpin loop structure. Thus, the double stranded molecule contained in the inventive composition may take the general formula 5′-[A]-[B]-[A′]-3′, wherein [A] is a polynucleotide strand which includes the sense strand sequence of a target sequence specifically hybridizing to an mRNA or a cDNA of the RQCD1 gene, the GIGYF1 gene or the GIGYF2 gene. Herein, the polynucleotide strand which includes the sense strand sequence of a target sequence specifically hybridizing to an mRNA or a cDNA of the RQCD1 gene, the GIGYF1 gene or the GIGYF2 gene, may be referred to as “sense strand”. In preferred embodiments, [A] is the sense strand; [B] is a single stranded polynucleotide consisting of 3 to 23 nucleotides; and [A′] is a polynucleotide strand which includes the antisense strand sequence of a target sequence specifically hybridizing to an mRNA or a cDNA of the RQCD1 gene, the GIGYF1 gene or the GIGYF2 gene (i.e., a sequence hybridizing to the target sequence of the sense strand [A]). Herein, the polynucleotide strand which includes the antisense strand sequence of a target sequence specifically hybridizing to an mRNA or a cDNA of the RQCD1 gene, the GIGYF1 gene or the GIGYF2 gene may be referred to as “antisense strand”. The region [A] hybridizes to [A′], and then a loop consisting of the region [B] is formed. The loop sequence may be preferably 3 to 23 nucleotides in length. The loop sequence, for example, can be selected from a group consisting of following sequences (www.ambion.com/techlib/tb/tb_{—}506.html):

CCC, CCACC, or CCACACC: Jacque J M et al., Nature 2002, 418: 435-8.

UUCG: Lee N S et al., Nature Biotechnology 2002, 20:500-5; Fruscoloni P et al., Proc Natl Acad Sci USA 2003, 100(4):1639-44.

UUCAAGAGA: Dykxhoorn D M et al., Nature Reviews Molecular Cell Biology 2003, 4:457-67.

‘UUCAAGAGA (“ttcaagaga” in DNA)’ is a particularly suitable loop sequence. Furthermore, loop sequence consisting of 23 nucleotides also provides an active siRNA (Jacque J M et al., Nature 2002, 418:435-8).

Exemplary hairpin siRNA suitable for the RQCD1 gene include:

5′-AAGAUCUAUCAGUGGAUCAAU-[b]-
AUUGAUCCACUGAUAGAUCUU-3′
(target sequence of SEQ ID NO: 8);
5′-AAGAUCUUGUUAGAUGACACU-[b]-
AGUGUCAUCUAACAAGAUCUU-3′
(target sequence of SEQ ID NO: 9);
5′-GAUCUAUCAGUGGAUCAAU-[b]-
AUUGAUCCACTGATAGAUC-3′
(target sequence of SEQ ID NO: 30)
and
5′-GAUCUUGUUAGAUGACACU-[b]-
TGUGUCAUCUAACAAGAUC-3′.
(target sequence of SEQ ID NO: 31)

Exemplary hairpin siRNA suitable for the GIGYF1 gene include:

5′-CCUUCCGAAGGGCUAGAGG-[b]-CCUCUAGCCCUUCGGAAGG-3′.
(target sequence of SEQ ID NO: 32)

Exemplary hairpin siRNA suitable for the GIGYF2 gene include:

5′-CAAGAUACCUUCAGACCUU-[b]-AAGGUCUGAAGGUTUCUUG-3′
(target sequence of SEQ ID NO: 33)

Other nucleotide sequences of suitable double-stranded molecules for the present invention can be designed using an siRNA design computer program available from the Ambion website (www.ambion.com/techlib/misc/siRNA_finder.html). The computer program selects nucleotide sequences for double-stranded molecule synthesis based on the following protocol.

Selection of Target Sites for Double-Stranded Molecules:

1. Beginning with the AUG start codon of the object transcript, scan downstream for AA dinucleotide sequences. Record the occurrence of each AA and the 3′ adjacent 19 nucleotides as potential target sites. Tuschl et al. Genes Cev 1999, 13(24):3191-7 don't recommend designing siRNA to the 5′ and 3′ untranslated regions (UTRs) and regions near the start codon (within 75 nucleotides) as these may be richer in regulatory protein binding sites. UTR-binding proteins and/or translation initiation complexes may interfere with binding of the siRNA endonuclease complex.

2. Compare the potential target sites to the human genome database and eliminate from consideration any target sequences with significant homology to other coding sequences. The homology search can be performed using BLAST (Altschul S F et al., Nucleic Acids Res 1997, 25:3389-402; J Mol Biol 1990, 215:403-10.), which can be found on the NCBI server at: www.ncbi.nlm.nih.gov/BLAST/.

3. Select qualifying target sequences for synthesis. At Ambion, preferably several target sequences can be selected along the length of the gene to evaluate.

Standard techniques are known in the art for introducing a double-stranded molecule into cells. For example, a double-stranded molecule can be directly introduced into the cells in a form that is capable of binding to the mRNA transcripts. In these embodiments, the double-stranded molecules are typically modified as described bellow for antisense molecules. Other modifications are also available, for example, cholesterol-conjugated double-stranded molecule have shown improved pharmacological properties (Song et al., Nature Med 2003, 9:347-51). These conventionally used techniques may also be applied for the double-stranded molecules contained in the present compositions.

Alternatively, a DNA encoding the double-stranded molecule may be carried in a vector (hereinafter, also referred to as ‘siRNA vector’) and the double-stranded molecule may be contained in the present composition in the form of vector which enables expression of the double-stranded molecule in vivo. Such vectors may be produced, for example, by cloning a portion of the target sequence sufficient to inhibit the in vivo expression of the target gene into an expression vector having operatively-linked regulatory sequences (e.g., a RNA polymerase III transcription unit from the small nuclear RNA (snRNA) U6 or the human H1 RNA promoter) flanking the sequence in a manner that allows for expression (by transcription of the DNA molecule) of both strands (Lee N S et al., Nature Biotechnology 2002, 20: 500-5). For example, an RNA molecule that is antisense to mRNA of the target gene is transcribed by a first promoter (e.g., a promoter sequence 3′ of the cloned DNA) and an RNA molecule that is the sense strand for the mRNA of the target gene is transcribed by a second promoter (e.g., a promoter sequence 5′ of the cloned DNA). The sense and antisense strands hybridize in vivo to generate the double-stranded molecule construct for silencing the expression of the target gene. Alternatively, the sense and antisense strands may be transcribed together with the help of one promoter. In this case, the sense and antisense strands may be linked via a polynucleotide sequence to form a single-stranded construct having secondary structure, e.g., hairpin.

Thus, the present pharmaceutical composition for treating or preventing cancer may include either the double-stranded molecule (e.g., siRNA) or a vector expressing the double-stranded molecule in vivo. In particular, the present invention provides pharmaceutical compositions for treating or preventing cancer that include a double-stranded molecule that inhibits the expression of the RQCD1 gene, the GIGYF1 gene or the GIGYF2 gene, or a vector expressing the double-stranded molecule in vivo.

Further, the present invention also provides pharmaceutical compositions for inhibiting cancer cell proliferation, such composition including a double-stranded molecule which inhibits the expression of the RQCD1 gene, the GIGYF1 gene or the GIGYF2 gene, or a vector expressing the double-stranded molecule in vivo.

Further, the present invention also provides pharmaceutical compositions for inhibiting Akt phosphorylation, such composition including a double-stranded molecule which inhibits the expression of the RQCD1 gene, the GIGYF1 gene or the GIGYF2 gene, or a vector expressing the double-stranded molecule in vivo.

For introducing the double-stranded molecule vector into the cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent. Therefore, the present pharmaceutical composition may further include such transfection-enhancing agents.

In another embodiment, the present invention also provides the use of the double-stranded nucleic acid molecules of the present invention or vector encoding thereof in manufacturing a pharmaceutical composition for treating a cancer expressing the RQCD1, GIGYF1 or GIGYF2 gene. For example, the present invention relates to a use of double-stranded nucleic acid molecule that inhibits the expression of RQCD1, GIGYF1 or GIGYF2 gene in a cell that over-expresses the gene, wherein the molecule includes a sense strand and an antisense strand complementary thereto, hybridized to each other to form the double-stranded nucleic acid molecule and targets to a sequence of SEQ ID NOs: 8, 9, 30, 31, 32, or 33 for manufacturing a pharmaceutical composition for treating a cancer expressing the RQCD1, GIGYF1 or GIGYF2 gene.

Alternatively, the present invention further provides the double-stranded nucleic acid molecules of the present invention for use in treating a cancer expressing the RQCD1, GIGYF1 or GIGYF2 gene.

Alternatively, the present invention further provides a method or process for manufacturing a pharmaceutical composition for treating a cancer expressing the RQCD1, GIGYF1 or GIGYF2 gene, wherein the method or process includes step formulating a pharmaceutically or physiologically acceptable carrier with a double-stranded nucleic acid molecule inhibiting the expression of RQCD1, GIGYF1 or GIGYF2 gene in a cell, which over-expresses the gene, wherein the molecule includes a sense strand and an antisense strand complementary thereto, hybridized to each other to form the double-stranded nucleic acid molecule and targets to a sequence of SEQ ID NOs: 8, 9, 30, 31, 32, or 33 as active ingredients.

In another embodiment, the present invention also provides a method or process for manufacturing a pharmaceutical composition for treating a cancer expressing the RQCD1, GIGYF1 or GIGYF2 gene, wherein the method or process includes step for admixing an active ingredient with a pharmaceutically or physiologically acceptable carrier, wherein the active ingredient is a double-stranded nucleic acid molecule inhibiting the expression of RQCD1, GIGYF1 or GIGYF2 gene in a cell, which overexpresses the gene, wherein the molecule includes a sense strand and an antisense strand complementary thereto, hybridized to each other to form the double-stranded nucleic acid molecule and targets to a sequence of SEQ ID NOs: 8, 9, 30, 31, 32, or 33.

IV-3. Pharmaceutical Compositions Including Antisense Nucleic Acids

Antisense nucleic acids targeting the RQCD1 gene, the GIGYF1 gene or the GIGYF2 gene can be used to reduce the expression level of the gene that is up-regulated in cancerous cells including breast cancer cells, lung cancer cells and esophageal cancer cells, particularly breast cancer cells. Such antisense nucleic acids are useful for the treatment of cancer, in particular breast cancer and thus are also encompassed by the present invention. An antisense nucleic acid acts by binding to the nucleotide sequence of the RQCD1 gene, the GIGYF1 gene or the GIGYF2 gene, or mRNAs corresponding thereto, thereby inhibiting the transcription or translation of the gene, promoting the degradation of the mRNAs, and/or inhibiting the expression of the protein encoded by the gene.

Thus, as a result, an antisense nucleic acid inhibits the RQCD1 protein, the GIGYF1 protein or the GIGYF2 protein to function in the cancerous cell. Herein, the phrase “antisense nucleic acids” refers to nucleotides that specifically hybridize to a target sequence and includes not only nucleotides that are entirely complementary to the target sequence but also that include mismatches of one or more nucleotides. For example, the antisense nucleic acids of the present invention include polynucleotides that have a homology of at least 70% or higher, preferably of at least 80% or higher, more preferably of at least 90% or higher, even more preferably of at least 95% or higher over a span of at least 15 continuous nucleotides of the RQCD1 gene, the GIGYF1 gene or the GIGYF2 gene or the complementary sequence thereof. Algorithms known in the art can be used to determine such homology.

Antisense nucleic acids of the present invention act on cells producing proteins encoded by the RQCD1 gene, the GIGYF1 gene or the GIGYF2 gene by binding to the DNA or mRNA of the gene, inhibiting their transcription or translation, promoting the degradation of the mRNA, and inhibiting the expression of the protein, finally inhibiting the protein to function.

Antisense nucleic acids of the present invention can be made into an external preparation, such as a liniment or a poultice, by admixing it with a suitable base material which is inactive against the nucleic acids.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. An antisense-mounting medium can also be used to increase durability and membrane-permeability. Examples include, but are not limited to, liposomes, poly-L-lysine, lipids, cholesterol, lipofectin, or derivatives of these. These can be prepared by following known methods.

The antisense nucleic acids of the present invention inhibit the expression of the RQCD1 gene, the GIGYF1 gene or the GIGYF2 gene and are useful for suppressing the biological activity of the protein. In addition, expression-inhibitors, including antisense nucleic acids of the present invention, are useful in that they can inhibit the biological activity of the RQCD1 protein, the GIGYF1 protein or the GIGYF2 protein. The antisense nucleic acids of present invention also include modified oligonucleotides. For example, thioated oligonucleotides may be used to confer nuclease resistance to an oligonucleotide.

IV-4. Pharmaceutical Compositions Including Antibodies

The function of a gene product of the RQCD1 gene, the GIGYF1 gene or the GIGYF2 gene which is over-expressed in cancers, in particular breast cancer, lung cancer and esophageal cancer, in more particular breast cancer can be inhibited by administering a compound that binds to or otherwise inhibits the function of the gene products. An antibody against the RQCD1 polypeptide, the GIGYF1 polypeptide or the GIGYF2 polypeptide can be mentioned as such a compound and can be used as the active ingredient of a pharmaceutical composition for treating or preventing cancer.

The present invention relates to the use of antibodies against a protein encoded by the RQCD1 gene, the GIGYF1 gene or the GIGYF2 gene, or fragments of the antibodies. As used herein, the term “antibody” refers to an immunoglobulin molecule having a specific structure, that interacts (i.e., binds) only with the antigen that was used for synthesizing the antibody (i.e., the gene product of an up-regulated marker) or with an antigen closely related thereto. Molecules including the antigen that was used for synthesizing the antibody and molecules including the epitope of the antigen recognized by the antibody can be mentioned as closely related antigens thereto.

Furthermore, an antibody used in the present pharmaceutical compositions may be a fragment of an antibody or a modified antibody, so long as it binds to the protein encoded by the RQCD1 gene, the GIGYF1 gene or the GIGYF2 gene (e.g., an immunologically active fragment of anti-RQCD1 antibody, anti-GIGYF1 antibody or anti-GIGYF2 antibody). For instance, the antibody fragment may be Fab, F(ab′)₂, Fv, or single chain Fv (scFv), in which Fv fragments from H and L chains are ligated by an appropriate linker (Huston J S et al., Proc Natl Acad Sci USA 1988, 85:5879-83). Such antibody fragments may be generated by treating an antibody with an enzyme, such as papain or pepsin. Alternatively, a gene encoding the antibody fragment may be constructed, inserted into an expression vector, and expressed in an appropriate host cell (see, for example, Co M S et al., J Immunol 1994, 152:2968-76; Better M et al., Methods Enzymol 1989, 178:476-96; Pluckthun A et al., Methods Enzymol 1989, 178:497-515; Lamoyi E, Methods Enzymol 1986, 121:652-63; Rousseaux J et al., Methods Enzymol 1986, 121:663-9; Bird R E et al., Trends Biotechnol 1991, 9:132-7).

An antibody may be modified by conjugation with a variety of molecules, such as polyethylene glycol (PEG). The present invention includes such modified antibodies. The modified antibody can be obtained by chemically modifying an antibody. Such modification methods are conventional in the field.

Alternatively, the antibody used for the present invention may be a chimeric antibody having a variable region derived from a non-human antibody against the RQCD1 polypeptide, the GIGYF1 polypeptide or the GIGYF2 polypeptide and a constant region derived from a human antibody, or a humanized antibody, including a complementarity determining region (CDR) derived from a non-human antibody, a frame work region (FR) and a constant region derived from a human antibody. Such antibodies can be prepared by using known technologies. Humanization can be performed by substituting rodent CDRs or CDR sequences for the corresponding sequences of a human antibody (see e.g., Verhoeyen et al., Science 1988, 239:1534-6). Accordingly, such humanized antibodies are chimeric antibodies, wherein substantially less than an intact human variable domain has been substituted by the corresponding sequence from a non-human species.

Complete human antibodies including human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom et al., J Mol Biol 1992, 227:381-8). Similarly, human antibodies can be made by introducing human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; and 5,661,016.

When the obtained antibody is to be administered to the human body (antibody treatment), a human antibody or a humanized antibody is preferable for reducing immunogenicity.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F. F. (Pharmacia).

Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

V. Methods for Treating or Preventing Cancer:

Cancer therapies directed at specific molecular alterations that occur in cancer cells have been validated through clinical development and regulatory approval of anti-tumor pharmaceuticals such as trastuzumab (Herceptin) for the treatment of advanced cancers, imatinib mesylate (Gleevec) for chronic myeloid leukemia, gefitinib (Iressa) for non-small cell lung cancer (NSCLC), and rituximab (anti-CD₂₀ mAb) for B-cell lymphoma and mantle cell lymphoma (Ciardiello F et al., Clin Cancer Res 2001, 7:2958-70, Review; Slamon D J et al., N Engl J Med 2001, 344:783-92; Rehwald U et al., Blood 2003, 101:420-4; Fang G et al., Blood 2000, 96:2246-53). These drugs are clinically effective and better tolerated than traditional anti-tumor agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L, Oncology 2002, 63 Suppl 1:47-56; Klejman A et al., Oncogene 2002, 21:5868-76). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid molecules as therapy to counteract aberrant expression of the differentially expressed genes or aberrant activity of their gene products.

Diseases and disorders that are characterized by increased (relative to a subject not suffering from the disease or disorder) expression levels or biological activities of genes and gene products, respectively, may be treated with therapeutics that antagonize (i.e., reduce or inhibit) activity of the over-expressed gene. Therapeutics that antagonize activity can be administered therapeutically or prophylactically.

Accordingly, therapeutics that may be utilized in the context of the present invention include, e.g., (i) a polypeptide of the over-expressed RQCD1 gene, GIGYF1 gene or GIGYF2 gene, or analogs, derivatives, fragments or homologs thereof; (ii) antibodies against the over-expressed gene or gene products; (iii) nucleic acids encoding the over-expressed gene; (iv) antisense nucleic acids or nucleic acids that are “dysfunctional” (i.e., due to a heterologous insertion within the nucleic acids of over-expressed gene); (v) double-stranded molecules (e.g., siRNA); or (vi) modulators (i.e., inhibitors, antagonists that alter the interaction between an over-expressed polypeptide and its binding partner). The dysfunctional antisense molecules are utilized to “knockout” endogenous function of a polypeptide by homologous recombination (see, e.g., Capecchi, Science 1989, 244: 1288 92).

Increased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods of the present invention may include the step of contacting a cell with an agent that modulates one or more of the activities of the RQCD1 gene, the GIGYF1 gene or the GIGYF2 gene products. Examples of agent that modulates protein activity include, but are not limited to, nucleic acids, proteins, naturally occurring cognate ligands of such proteins, peptides, peptidomimetics, and other small molecule.

Thus, the present invention provides methods for treating or alleviating a symptom of cancer, or preventing cancer in a subject by decreasing the expression of the RQCD1 gene, the GIGYF1 gene or the GIGYF2 gene or the activity of the gene product. The present method is particularly suited for treating or preventing breast cancer.

Suitable therapeutics can be administered prophylactically or therapeutically to a subject suffering from or at risk of (or susceptible to) developing cancers. Such subjects can be identified by using standard clinical methods or by detecting an aberrant expression level (“up-regulation” or “over-expression”) of the RQCD1 gene, the GIGYF1 gene or the GIGYF2 gene or aberrant activity of the gene product.

According to an aspect of the present invention, an agent screened through the present method may be used for treating or preventing cancer. Methods well known to those skilled in the art may be used to administer the agents to patients, for example, as an intraarterial, intravenous, or percutaneous injection or as an intranasal, transbronchial, intramuscular, or oral administration. If said agent is encodable by a DNA, the DNA can be inserted into a vector for gene therapy and the vector administered to a patient to perform the therapy.

The dosage and methods for administration vary according to the body-weight, age, sex, symptom, condition of the patient to be treated and the administration method; however, one skilled in the art can routinely select suitable dosage and administration method.

For example, although the dose of an agent that binds to an RQCD1 polypeptide, a GIGYF1 polypeptide or a GIGYF2 polypeptide and regulates the activity of the polypeptide depends on the aforementioned various factors, the dose is generally about 0.1 mg to about 100 mg per day, preferably about 1.0 mg to about 50 mg per day and more preferably about 1.0 mg to about 20 mg per day, when administered orally to a normal adult human (60 kg weight).

When administering the agent parenterally, in the form of an injection to a normal adult human (60 kg weight), although there are some differences according to the patient, target organ, symptoms and methods for administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

Similarly, a pharmaceutical composition of the present invention may be used for treating or preventing cancer. Methods well known to those skilled in the art may be used to administer the compositions to patients, for example, as an intraarterial, intravenous, or percutaneous injection or as an intranasal, transbronchial, intramuscular, or oral administration.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age, body weight and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

In particular, an antisense nucleic acid against the RQCD1 gene, the GIGYF1 gene or the GIGYF2 gene can be given to the patient by direct application onto the ailing site or by injection into a blood vessel so that it will reach the site of ailment. The dosage of the antisense nucleic acid derivatives of the present invention can be adjusted suitably according to the patient's condition and used in desired amounts. For example, a dose range of 0.1 to 100 mg/kg, preferably 0.1 to 50 mg/kg can be administered.

VI. Double-Stranded Molecules and Vectors Encoding Them

Herein, an siRNA including either of the sequences of SEQ ID NOs: 8, 9, 30 or 31 was demonstrated to suppress cell growth or viability of cells expressing the RQCD1 gene. Therefore, double-stranded molecules including any of these sequences and vectors expressing the molecules are considered to serve as preferable pharmaceutics for treating or preventing diseases which involve the proliferation of RQCD1 gene expressing cells, for example, breast cancer, lung cancer and esophageal cancer, particularly breast cancer. Thus, according to an aspect, the present invention provides double-stranded molecules including the target sequence selected from the group consisting of SEQ ID NOs: 8, 9, 30 and 31 and vectors expressing the molecules. More specifically, the present invention provides a double-stranded molecule, when introduced into a cell expressing the RQCD1 gene, inhibits expression of the gene, which molecule includes a sense strand and an antisense strand, wherein the sense strand includes a nucleotide sequence selected from the group consisting of SEQ ID NOs: 8, 9, 30 and 31 as a target sequence, and the antisense strand includes a nucleotide sequence complementary to the target sequence of the sense strand so that the sense and antisense strands hybridize to each other to form the double-stranded molecule.

Herein, siRNAs including the sequence of SEQ ID NOs: 32 or 33 were demonstrated to suppress the expression of the GIGYF1 gene and the GIGYF2 gene respectively, and lead to suppression of Akt phosphorylation. Therefore, double-stranded molecules including these sequences and the vectors expressing the molecules are considered to serve as preferable pharmaceutics for inhibiting Akt phosphorylation, and likely to be useful for inhibiting cancer cell proliferation, further treating or preventing cancers. Thus, the present invention also provides double-stranded molecules including the target sequence selected from the group consisting of SEQ ID NOs: 32 and 33 and vectors expressing the molecules. More specifically, the present invention provides a double-stranded molecule, when introduced into a cell expressing the GIGYF1 gene or the GIGYF2 gene, inhibits expression of the gene, which molecule includes a sense strand and an antisense strand, wherein the sense strand includes a nucleotide sequence selected from the group consisting of SEQ ID NOs: 32 and 33 as a target sequence, and the antisense strand includes a nucleotide sequence complementary to the target sequence of the sense strand so that the sense and antisense strands hybridize to each other to form the double-stranded molecule.

The target sequence for the RQCD1 gene included in the sense strand may consist of a sequence of a portion of SEQ ID NO: 10 that is less than about 500, 400, 300, 200, 100, 75, 50 or 25 contiguous nucleotides. For example, the target sequence may be from about 19 to about 25 contiguous nucleotides from the nucleotide sequence of SEQ ID NO: 10. The present invention is not limited thereto, but suitable target sequences include the nucleotide sequences selected from the group consisting of SEQ ID NOs: 8, 9, 30 and 31.

The target sequence for the GIGYF1 gene or the GIGYF2 gene included in the sense strand may consist of a sequence of a portion of SEQ ID NO: 35 or 37 that is less than about 500, 400, 300, 200, 100, 75, 50 or 25 contiguous nucleotides. For example, the target sequence may be from about 19 to about 25 contiguous nucleotides from the nucleotide sequence of SEQ ID NO: 35 or 37. The present invention is not limited thereto, but suitable target sequences include the nucleotide sequences selected from the group consisting of SEQ ID NOs: 32 and 33.

The double-stranded molecule of the present invention may be composed of two polynucleotide constructs, i.e., a polynucleotide including the sense strand and a polynucleotide including the antisense strand. Alternatively, the molecule may be composed of one polynucleotide construct; i.e., a polynucleotide including both the sense strand and the antisense strand, wherein the sense and antisense strands are linked via a single-stranded polynucleotide which enables hybridization of the target sequences within the sense and antisense strands by forming a hairpin structure. Herein, the single-stranded polynucleotide may also be referred to as “loop sequence” or “single-strand”. The single-stranded polynucleotide linking the sense and antisense strands may consist of 3 to 23 nucleotides. See under the item of “IV-2. Pharmaceutical compositions including double-stranded molecules” for more details on the double-stranded molecule of the present invention.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 21-O-methyl ribonucleotides (especially on the sense strand of a double-stranded molecule), 2′-deoxy-fluoro ribonucleotides, 2′-deoxy ribonucleotides, “universal base” nucleotides, 5′-C— methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule including both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention include following combinations.

sense strand:
5′-[---DNA---]-3′
3′-(RNA)-[DNA]-5′:
antisense strand,
sense strand:
5′-(RNA)-[DNA]-3′
3′-(RNA)-[DNA]-5′:
antisense strand,
and
sense strand:
5′-(RNA)-[DNA]-3′
3′-(---RNA---)-5′:
antisense strand.

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the context of the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

Alternatively, the present invention provides vectors including each of a combination of polynucleotide having a sense strand nucleic acid and an antisense strand nucleic acid, wherein said sense strand nucleic acid includes nucleotide sequence of SEQ ID NOs: 8, 9, 30, 31, 32, or 33, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the RQCD1, GIGYF1 or GIGYF2, inhibits expression of said gene. Preferably, the polynucleotide is an oligonucleotide of between about 19 and 25 nucleotides in length (e.g., contiguous nucleotides from the nucleotide sequence of SEQ ID NO: 10, 35, or 37). More preferably, the combination of polynucleotide includes a single nucleotide transcript having the sense strand and the antisense strand linked via a single-stranded nucleotide sequence. More preferably, the combination of polynucleotide has the general formula 5′-[A]-[B]-[A]-3′, wherein [A] is a nucleotide sequence including SEQ ID NO: 8, 9, 30, 31, 32, or 33; [B] is a nucleotide sequence consisting of about 3 to about 23 nucleotide; and [A′] is a nucleotide sequence complementary to [A].

Vectors of the present invention can be produced, for example, by cloning RQCD1, GIGYF1 or GIGYF2 sequence into an expression vector so that regulatory sequences are operatively-linked to RQCD1, GIGYF1 or GIGYF2 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas KR & Capecchi MR, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687). The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

In the present invention, the inhibitory nucleic acids can be administered to the subject either as a naked nucleic acid, in conjunction with a delivery reagent, or as a recombinant plasmid or viral vector that expresses the inhibitory nucleic acids. Suitable delivery reagents for administration in conjunction with the present inhibitory nucleic acids include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the inhibitory nucleic acids to a particular tissue, such as retinal or tumor tissue, and can also increase the blood half-life of the inhibitory nucleic acids. Liposomes suitable for use in the context of the present invention may be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present inhibitory nucleic acids include a ligand molecule that can deliver the liposome to the cancer site. Ligands which bind to receptors prevalent in tumor cells, such as monoclonal antibodies that bind to tumor antigens, are preferred.

Particularly preferably, the liposomes encapsulating the present inhibitory nucleic acids are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-in-hibition moieties can deliver the present inhibitory nucleic acids to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.l. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH3 and a solvent mixture such as tetrahydrofuran and water in a 30:12 ratio at 60 degree C.

Vectors expressing inhibitory nucleic acids of the present invention are discussed above. Such vectors expressing at least one inhibitory nucleic acids of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express inhibitory nucleic acids of the invention, to an area of cancer in a patient are within the skill of the art.

The inhibitory nucleic acids of the invention can be administered to the subject by any means suitable for delivering the inhibitory nucleic acids into cancer sites. For example, the inhibitory nucleic acids can be administered by gene gun, electroporation, or by other suitable parenteral or enteral administration routes.

Suitable enteral administration routes include oral, rectal, or intranasal delivery.

Suitable parenteral administration routes include intravesical and intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps); direct application to the area at or near the site of cancer, for example by a catheter or other placement device (e.g., a retinal pellet or a suppository or an implant including a porous, non-porous, or gelatinous material); and inhalation. It is preferred that injections or infusions of the inhibitory nucleic acids or vector be given at or near the site of the cancer.

The inhibitory nucleic acids of the invention can be administered in a single dose or in multiple doses. Where the administration of the inhibitory nucleic acids of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the inhibitory nucleic acids of the invention to a given subject. For example, the inhibitory nucleic acids can be administered to the subject once, for example, as a single injection or deposition at or near the cancer site. Alternatively, the inhibitory nucleic acids can be administered once or twice daily to a subject for a period of from about three to about twenty-eight days, more preferably from about seven to about ten days. In a preferred dosage regimen, the inhibitory nucleic acids are injected at or near the site of cancer once a day for seven days. Where a dosage regimen includes multiple administrations, it is understood that the effective amount of an inhibitory nucleic acids administered to the subject can include the total amount of an inhibitory nucleic acids administered over the entire dosage regimen.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

### Example 1

### I. Materials and Methods

1. Cell Lines and Clinical Samples

Human breast cancer cell lines BT-549, HCC1937, MCF-7, MDA-MB-435S, MDA-MB-231, SKBR3, T47D, YMB1, and BSY-1, as well as immortalized human mammary cell-line HBL100 are purchased from American Type Culture Collection (Rockville, Md.), and cultured under the recommendations of their respective depositors. Human mammalian epithelial cell (HMEC) is purchased from Cambrex Bio Science (Walkersville, Md.). HBC4 and HBC5 cell lines and are kindly gifted by Dr. Yamori (Division of Molecular Pharmacology, Cancer Chemotherapy Center, Japanese Foundation for Cancer Research, Tokyo, Japan). All cells were cultured in the appropriate mediums, i.e., RPMI 1640 (Sigma-Aldrich) for HBC4, HBC5, HCC1937, T47D, and YMB1 (with 2 mM L-glutamine); DMEM (Sigma-Aldrich) for HBL100; EMEM (Sigma-Aldrich) for BT-549 and MCF-7 (with 0.001% insulin); McCoy (Sigma-Aldrich) for SKBR3 (with 1.5 mM L-glutamine); L-15 (Roche) for MDA-MB-231 and MDA-MB-4355; MEGM (Cambrex Bio Science) for HMEC. Each medium was supplemented with 10% fetal bovine serum (Cansera International) and 1% antibiotic/antimycotic solution (Sigma-Aldrich). MDA-MB-231 and MDA-MB-435S cells were maintained at 37 degree C. in atmosphere of humidified air without CO2. Other cell lines were maintained at 37 degree C. in atmosphere of humidified air with 5% CO2. Tissue samples from surgically resected breast cancers and their corresponding clinical information were obtained from Department of Breast Surgery, Cancer Institute Hospital, Tokyo, with a written informed consent.

2. Semi-Quantitative RT-PCR

Total RNA was extracted from each of microdissected breast cancer clinical samples and normal ductal cells, and then performed T7-based amplification and reverse transcription as described previously (Nishidate at al., 2004). Appropriate dilutions of each single-stranded cDNA was prepared for subsequent PCR by monitoring the amplification of beta actin as a quantitative internal control. The PCR primer sequences were

(SEQ ID NO: 1)
5′-CGACCACTTTGTCAAGCTCA-3′
and
(SEQ ID NO: 2)
5′-GGTTGAGCACAGGGTACTTTATT-3′
for beta actin;
and
(SEQ ID NO: 3)
5′-AGACCCTAAAGATCGTCCTTCTG-3′
and
(SEQ ID NO: 4)
5′-GTGTTTTAAGTCAGCATGAGCAG-3′
for RQCD1.

3. Northern Blot Analysis

Total RNAs were extracted from all breast cancer cell lines using RNeasy kit (QIAGEN) according to the instructions from the manufacturer. After the treatment with DNase I, mRNA was isolated with mRNA purification kit (GE Healthcare) following the instructions of the manufacturer. One microgram each of mRNA isolated from normal adult human mammary gland (Biochain), lung, heart, liver, kidney, and bone marrow (BD Biosciences) was separated on 1% denaturing agarose gels and transferred to a nylon membrane by the capillary blotting. Human multiple-tissue northern blot membrane (BD Biosciences) were hybridized with [³²P]-dCTP labeled RQCD1 cDNA. Prehybridization, hybridization, and washing were performed similarly as described previously (Hirota E, et al. (2006) Int J Oncol 29:799-827). Following washing step, membranes were autoradiographed with intensity enhancing screens for 14 days at −80 degree C. specific probe for RQCD1 (241 bp) was prepared by RT-PCR using the primer set 5′-AGACCCTAAAGATCGTCCTTCTG-3′ (SEQ ID NO: 3) and 5′-GTGTTTTAAGTCAGCATGAGCAG-3′ (SEQ ID NO: 4), and was radioactively labeled with megaprime DNA labeling system (GE Healthcare).

4. Immunocytochemistry

RQCD1 cDNA was prepared by PCR amplification. The PCR product was inserted into pCAGGS mammalian expression vector designated for N-terminus hemagglutinin (HA)-tagged RQCD1. pCAGGS-HA-RQCD1 expression vector was transfected to HEK293, HBC4 or BT549 using FuGENE6 (Roche) according to the instructions from the manufacturer. Following 24 h incubation, cells were fixed with 4% formaldehyde for 15 min, and rendered permeable with 0.1% Triton X-100 at 4 degree C. for 3 min. Subsequently, the cells were incubated in 3% BSA for 1 hour for blocking, and incubated with 3F10 anti-HA mouse monoclonal antibody (Roche) diluted at 1:500. After washing with PBS(−), the cells were incubated with Alexa 594-conjugated anti-mouse antibody (Molecular Probes) diluted at 1:500. Nuclei were counterstained with 4′,6′-diamidine-2′-phenylindole dihydrochloride (DAPI). Fluorescent images were obtained under a TCS SP2 AOBS confocal microscope (Leica).

5. Knockdown of RQCD1 with U6-siRNA Vector System

A vector-based RNA interference (RNAi) expression system was established using psiU6BX3.0 small interfering RNA (siRNA) expression vector (Hirota E, et al. (2006) Int J Oncol 29:799-827). An siRNA expression vector against RQCD1 was prepared by cloning of double-stranded oligonucleotides into the BbsI site in the psiU6BX3.0 vector. The target sequences of the siRNA were as follows:

si-#1,
(SEQ ID NO.: 8)
5′-AAGATCTATCAGTGGATCAAT-3′;
si-#2,
(SEQ ID NO.: 9)
5′-AAGATCTTGTTAGATGACACT-3′.

All of the constructs were confirmed by DNA sequencing. Human breast cancer cell lines, HBC4 and BT549, were plated onto 6-well plates (0.4×10⁵) and transfected with psiU6BX3.0-mock (without insertion) or psiU6BX3.0-RQCD1 (si-#1, si-#2 and constructs including three-base substitutions in si-#1) using FuGENE6 reagent (Roche) according to the instructions from the manufacturer. At 24 hours after the transfection, cells are reseeded for colony formation assay, RT-PCR and MTT assay. The psiU6BX3.0-introduced HBC4 and BT549 cells were selected with a culture medium containing 0.5 mg/ml of Geneticin (Invitrogen). Total RNA was extracted after the selection for 5 days and then evaluated the knockdown effect by semiquantitative RT-PCR using specific primer sets; 5′-ATGGAAATCCCATCACCATCT-3′ (SEQ ID NO: 5) and 5′-GGTTGAGCACAGGGTACTTTATT-3′ (SEQ ID NO: 2) for beta actin as an internal control, and 5′-GCCTTCATCATCCAAACATT-3′ (SEQ ID NO: 6) and 5′-GGCAAATATGTCTGCCTTGT-3′ (SEQ ID NO: 7) for RQCD1.

6. Establishment of HEK293 Cells Stably Expressing RQCD1

HA-tagged RQCD1 expression vector (pCAGGSnHC-RQCD1) or mock vector (pCAGGSnHC) was transfected into HEK293 cells using FuGENE6 transfection reagent (Roche). Transfected cells were incubated in the culture medium containing 0.9 mg/ml of neomycin (geneticin; Invitrogen). Three weeks later, 20 individual colonies were selected by limiting dilution and screened for RQCD1-stably-expressing clones. The expression level of HA-tagged RQCD1 was detected in each clone by western blot and immunohistochemical staining analyses using anti-HA monoclonal antibody (Sigma). Three independent clones were established and designated them as follows; HEK293-RQCD1-1, -2 and -3, and HEK293-Mock-1, -2 and -3.

7. Colony Formation Assay and MTT Assay

Transfectants expressing siRNA were grown for 14 days in selective medium containing geneticin, then fixed with 4% paraformaldehyde for 15 minutes before staining with Giemsa solution (Merck, Whitehouse Station, N.J.) to assess colony number. To quantify cell viability, MTT assays were done with cell counting kit-8 according to recommendations from the manufacturer (Wako, Osaka, Japan). Absorbance at 570 nm wavelength was measured with a Microplate Reader 550 (Bio-Rad). These experiments were done in triplicate.

### II. Results

1. Up-Regulation of RQCD1 in the Clinical Breast Cancer Cells

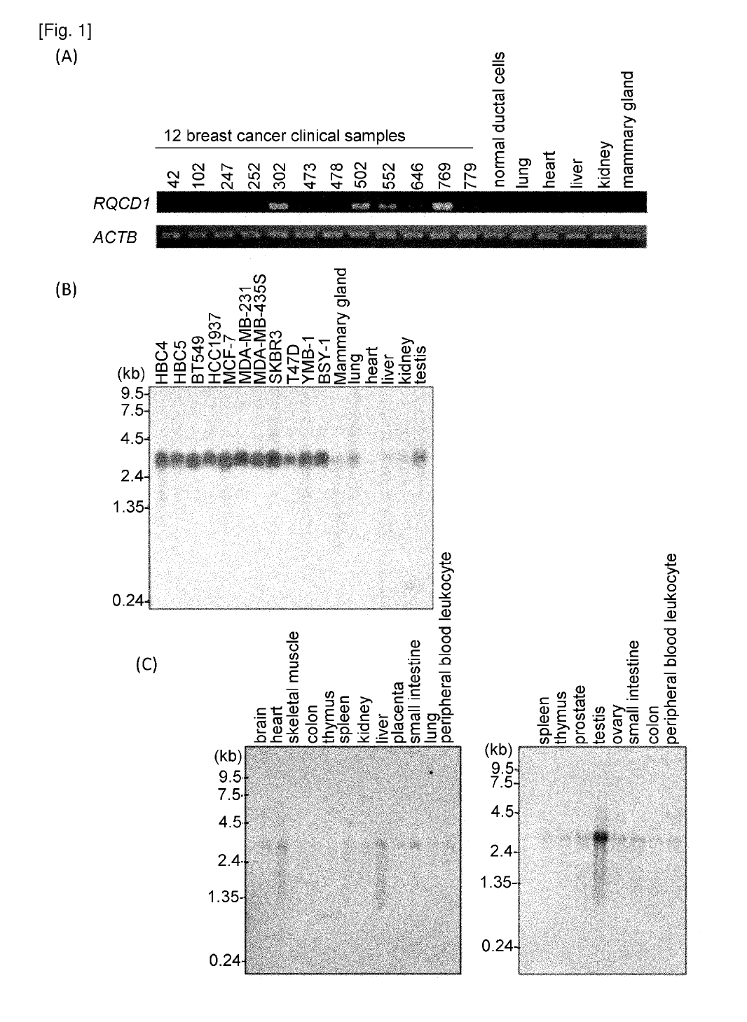

The up-regulation of RQCD1 (RCD1 required for cell differentiation 1 homolog (S. pombe)) gene (Genebank accession; NM_{—}005444) was validated in 4 of 12 clinical breast cancer cases by semiquantitative RT-PCR analysis as compared with normal mammary ductal cells or vital organs (FIG. 1A). Subsequent northern blot analysis using a RQCD1 cDNA fragment as a probe showed that an approximately 3.5-kb transcript of RQCD1 was significantly elevated in all of eleven breast cancer cell lines examined (FIG. 1B), compared with mammary gland. This transcript was most highly expressed in testis, but its expression was hardly detectable in any of the remaining normal organs (FIG. 1C), suggesting RQCD1 is a possible cancer-testis antigen.

2. Subcellular Localization of Exogenously Expressed RQCD1

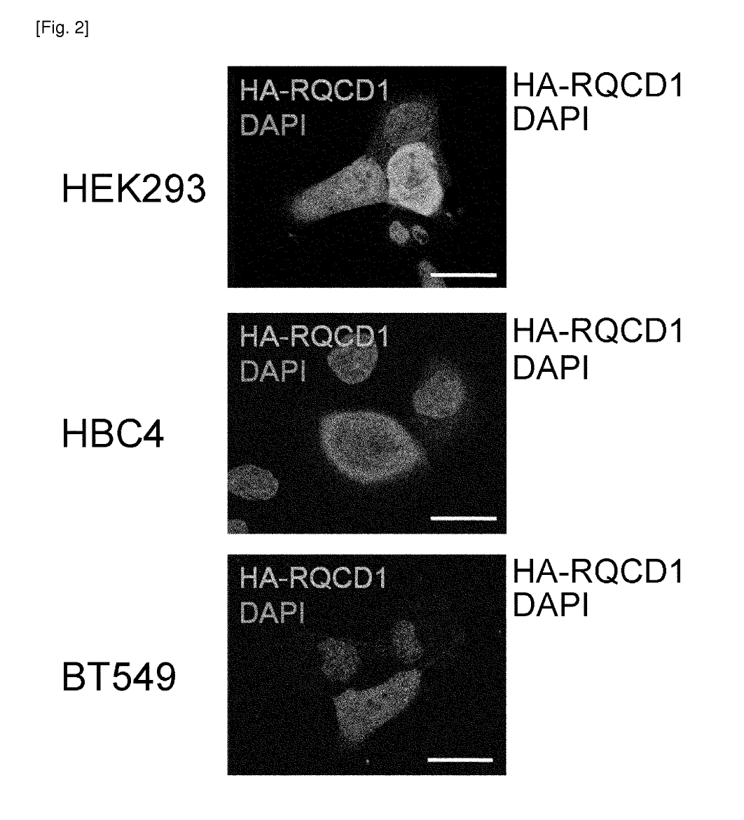

To characterize the biological role of RQCD1 protein, the subcellular localization of exogenously introduced RQCD1 was first examined in HEK293, HBC4 or BT549 by immunocytochemistry. Forty-eight hours after transfection with HA-tagged RQCD1 construct, exogenously expressed-RQCD1 protein was stained diffusely in both of cytoplasm and nucleus (FIG. 2)

3. Knockdown of RQCD1 Leads to Growth Inhibition for Breast Cancer Cell Lines

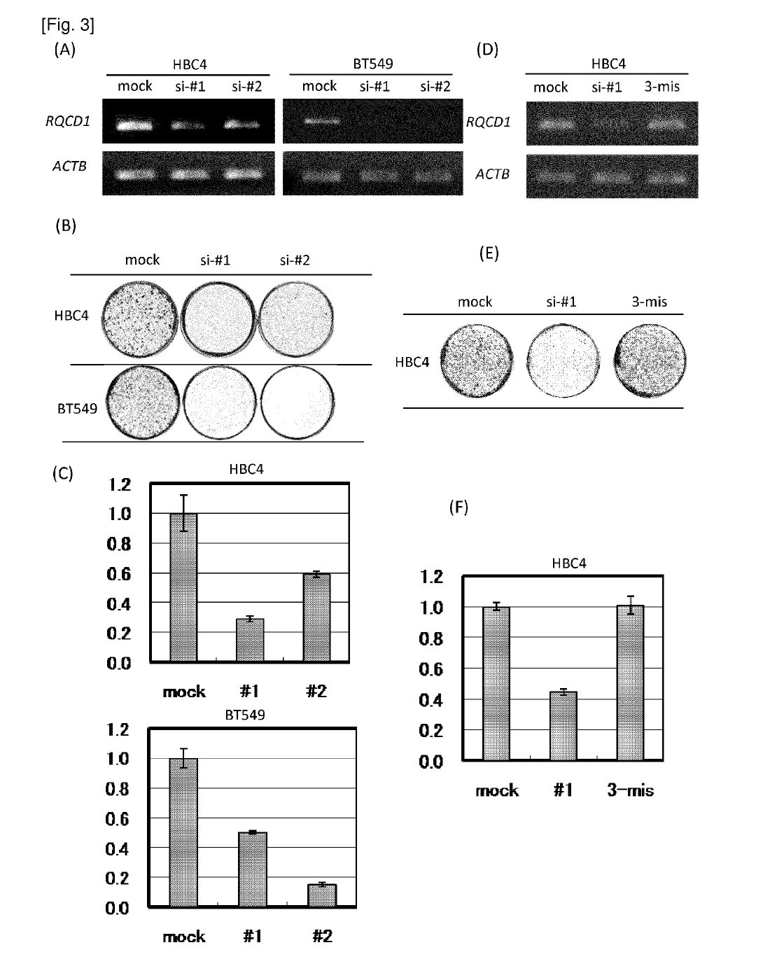

To investigate the biological significance of RQCD1 in the breast cancer cell, U6 promoter-based shRNA expression vectors targeting the sequences specific to RQCD1 was constructed, and transfected them into HBC4 and BT549, breast cancer cell lines, in which RQCD1 is highly expressed. Semiquantitative RT-PCR detected significant knockdown effect of RQCD1 expression in both cell lines transfected with psiU6BX-RQCD1-si#1 and si#2, compared with a control siRNA construct, psiU6BX-mock (FIG. 3A). In concordance with the knockdown effect on the transcript, MTT and colony formation assays revealed significantly growth suppression of breast cancer cells by the two siRNAs, si#1 and si#2 (FIG. 3B). Specificity of knockdown effect with 3-base mismatch siRNA was further evaluated, and no significant effect was observed in the case of 3-base mismatch siRNA (FIG. 3D), supporting the sequence specificity of the knockdown of RQCD1. This evidence strongly suggests that RQCD1 plays an important role in the breast cancer cell growth.

4. Constitutive Overexpression of RQCD1 Resulted in the Enhanced Cell Growth in HEK293

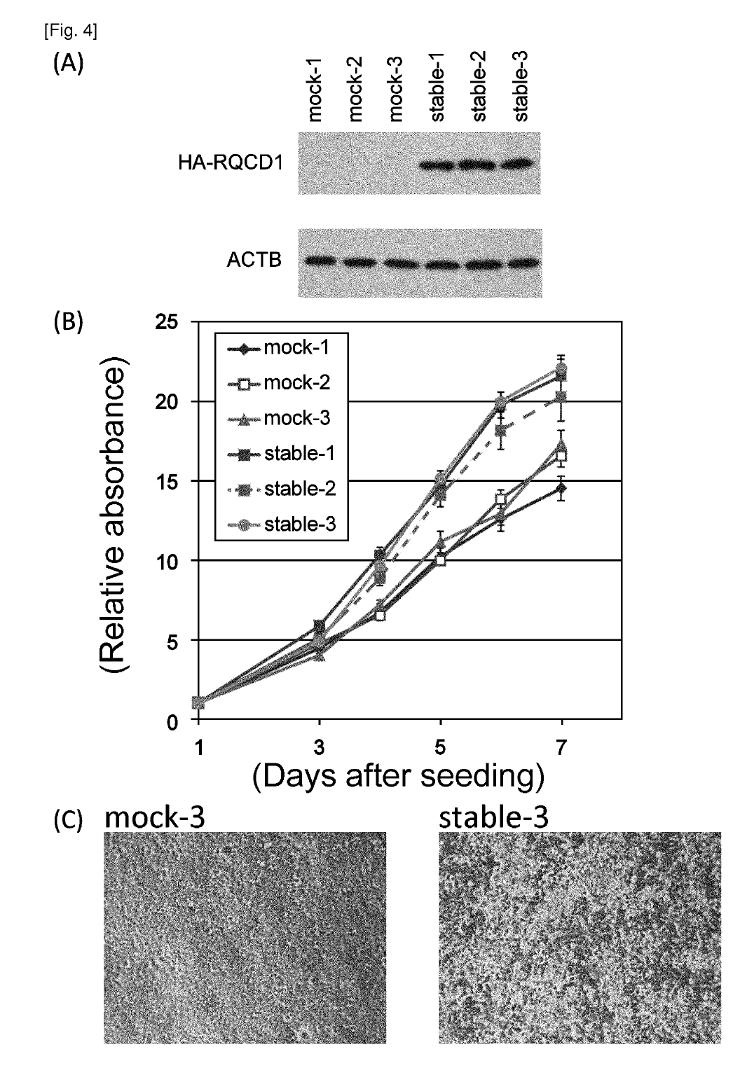

To further explore the growth promoting effect of RQCD1, HEK293-derivative cells were established that stably expressed exogenous RQCD1. The exogenous RQCD1 protein was confirmed as observed at high level and monoclonality in three stable cell lines by western blot analysis (FIG. 4A) and immunocytochemistry (data not shown). Subsequent MTT assays showed that the three RQCD1-stable derivative cells (RQCD1-1, -2, and -3) grew much faster than those transfected with mock plasmid (Mock-1, -2, and -3) (FIG. 4B), suggesting a growth-enhancing effect of RQCD1. In addition to the rapid growth multilayer-growth of these three HEK293-RQCD1 cells was observed after they reached at the confluence phase, indicating loss of the contact inhibition mechanism by RQCD1 introduction into HEK293 cells (FIG. 4C).

### Example 2

### I. Materials and Methods

1. Breast Cancer Cell Lines and Clinical Samples.

Human breast cancer cell lines, HCC-1937, BT-549, MCF-7, BSY-1, MDA-MB-435S, SKBR-3, T-47D, MDA-MB-231 and YMB-1, human normal ductal epithelial cell MCF10A, human embryonic kidney cell lines, HEK293 and HEK293T, were purchased from American Type Culture Collection (ATCC; Rockville, Md., USA). They were cultured under the recommendations of their respective depositors. HBC-4 and HBC-5 cell lines were kindly provided by Dr. Takao Yamori of Division of Molecular Pharmacology, Cancer Chemotherapy Center, Japanese Foundation for Cancer Research. Culture media for these cell lines were as follows, RPMI 1640 (Invitrogen, Carlsbad, Calif., USA) with 2 mM L-glutamine for HCC-1937, T-47D, SKBR-3, YMB-1, HBC-4 and HBC-5; EMEM (Invitrogen) with 0.001% insulin for MCF-7 and HEK293; L-15 (Sigma-Aldrich, St Louis, Mo., USA) for MDA-MB-231 and MDA-MB-4355; DMEM (Sigma-Aldrich) for HEK293T; MEGM (Lonza, Basel, Switzerland) with 13 mg/ml Bovine Pituitary Extract, 0.5 mg/ml hydrocortisone, 10 microgram/ml EGF, 5 mg/ml insulin and 100 ng/ml cholera toxin (Lonza) for MCF10A. Each medium except MEGM was supplemented with 10% fetal bovine serum (Cansera International, Ontario, Canada) and 1% antibiotic/antimycotic solution (Sigma-Aldrich). MDA-MB-231 and MDA-MB-4355 cells were maintained at 37 degree C. in atmosphere of humidified air without CO2. Other cell lines were maintained at 37 degree C. in atmosphere of humidified air with 5% CO₂. Tissue samples from surgically resected breast cancers and their corresponding clinical information were obtained from Department of Breast Surgery, Cancer Institute Hospital, Tokyo after obtaining written informed consent. This study including the use of all clinical materials described above was approved by individual institutional Ethical Committees.

2. Semiquantitative Reverse Transcription-PCR Analysis.

Total RNA was extracted from 12 microdissected clinical breast cancer cells and culture cell lines using RNeasy Kit according to the manufacture's protocol (GE Healthcare, Buckinghamshire, UK). Extracted RNAs and normal human tissue polyadenylate RNAs were treated with DNase I (Nippon Gene, Tokyo, Japan), and then were reversely transcribed using SuperScript First-Strand Synthesis System (Invitrogen). Appropriate dilutions of each single-stranded cDNA were prepared for subsequent PCR by monitoring bete-actin (ACTB) as an internal control. The PCR primer sequences were as follows;

(SEQ ID NO: 12)
5′-GGAACGGTGAAGGTGACAGC-3′
and
(SEQ ID NO: 13)
5′-ACCTCCCCTGTGTGGACTTG-3′
for ACTB,
(SEQ ID NO: 14)
5′-GGACTTGTTAGTTGGCTTCTGTC-3′
and
(SEQ ID NO: 15)
5′-GATCACTTCTCTTCAGGCTTGC-3′
for 3′-UTR region of RQCD1
(analysis of expression in
clinical samples and cell lines).
(SEQ ID NO: 16)
5′-CTGGCACAAGTGGATAGAGAAA-3′
and
(SEQ ID NO: 17)
5′-CAGAAGGCTCTTTGGATAGCTG-3′
for RQCD1
(analysis of it′s knocking-down effect),
(SEQ ID NO: 18)
5′-CAGCAGAGACACTCAACTTTGG-3′
and
(SEQ ID NO: 19)
5′-CTTCTTCGATGCTTCTTTG GTAA-3′
for GIGYF1,
and
(SEQ ID NO: 20)
5′-CGGCAGAGAAGAAATGTTAGC-3′
and
(SEQ ID NO: 21)
5′-GCTTTCTCCCTACTGATGTTGG-3′
for GIGYF2.

3.5′ and 3′ Rapid Amplification of cDNA Ends (5′ and 3′ RACE).

The 5′ and 3′ RACE experiments were carried out using SMART RACE cDNA Amplification Kit (Takara Clontech) according to the manufacturer's instructions. For the amplification of the 5′ and 3′ sequences of RQCD1 cDNA, gene-specific primers (5′-GCGGCAACCCTGTAATTCCCATAGAC-3′ (SEQ ID NO: 22) for 5′ RACE and 5′-GGAGGTGCTTGGGATTAAGGTGACAG-3′ (SEQ ID NO: 23) for 3′ RACE) and a universal primer mixture supplied in the kit were used. The cDNA template was synthesized from mRNA purified from HBC-4 breast cancer cells, using Superscript II Reverse Transcriptase (Invitrogen). The PCR products were cloned using TA cloning kit (Invitrogen) and sequences were determined by DNA sequencing (ABI3700; PE Applied Biosystems, Foster, Calif.).

4. Northern Blotting.

Breast cancer northern blot membranes were prepared as described previously (7). Human Multiple-Tissue Northern blot membrane (Takara Clontech, Kyoto, Japan) and breast cancer northern blot membranes were hybridized with [alpha³²P]-dCTP labeled cDNA probe for RQCD1, prepared by RT-PCR (see below) with megaprime DNA labeling system (GE Healthcare). Prehybridization, hybridization and washing were performed as described previously (Katagiri T, Ozaki K, Fujiwara T et al: Cloning, expression and chromosome mapping of adducin-like 70 (ADDL), a human cDNA highly homologous to human erythrocyte adducin. Cytogenet Cell Genet. 4: 90-95, 1996). The blots were autoradiographed with intensifying screens at −80 degree C. for 14 days. Specific probe for RQCD1 (283 bp) was prepared by RT-PCR using the primer set of 5′-GGACTTGTTAGTTGGCTTCTGTC-3′ (SEQ ID NO: 14) and 5′-GATCACTTCTCTTCAGGCTTGC-3′ (SEQ ID NO: 15).

5. Construction of Expression Vectors.

To expression vector constructs for RQCD1, GIGYF1 and GIGYF2, each entire coding sequence was amplified by PCR using KOD-Plus DNA polymerase (TOYOBO, Osaka, Japan). Primer sets were as follows; 5′-GGAATTCAATGCACAGCCTGGCGACGG-3′ (SEQ ID NO: 24) and 5′-GGACTCGAGCTGAGGGGGCAGGGGGATA-3′ (SEQ ID NO: 25) for RQCD1, 5′-GTTAAGTAGCGGCCGCTCATGGCAGCAGAGACACTCAAC-3′ (SEQ ID NO: 26) and 5′-CCGCTCGAGGTAGTCATCCACGCTCTC-3′ (SEQ ID NO: 27) for GIGYF 1, and 5′-GTTAAGTAGCGGCCGCTCATGGCAGCGGAAACGCAGAC-3′ (SEQ ID NO: 28) and 5′-CCGCTCGAGGTAGTCATCCAACGTCTC-3′ (SEQ ID NO: 29) for GIGYF2 (underlines indicate recognition sites of restriction enzymes). The PCR products were inserted into pCAGGSn3FC or pCAGGSnHC expression vector in frame with hemagglutinin (HA)-tag or Flag-tag at the C-terminus, respectively. DNA sequences of each construct were confirmed by DNA sequencing (ABI3700; PE Applied Biosystems).

6. Preparation of Anti-RQCD1 Polyclonal Antibody.

Plasmid designed to express the full-length RQCD1 with glutathione S-transferase (GST)-tag at the N-terminus was constructed using pGEX-6P-1 vector (GE Healthcare). The recombinant RQCD1 protein was expressed in BL21-CodonPlus-RIL Escherichia coli strain (Stratagene, La Jolla, Calif., USA) and purified using Glutathione Sepharose 4B (Zymed Laboratories, South San Francisco, Calif., USA), followed by digestion with PreScission Protease (GE Healthcare) to remove GST-tag according to the supplier's protocols. The purified recombinant protein was used for immunization of rabbits (SCRUM, Tokyo, Japan). The immune sera were subsequently purified on antigen affinity columns using Affi-gel 10 (Bio-Rad Laboratories, Hercules, Calif., USA) according to supplier's instructions.

7. Western Blotting.

To examine the expression of RQCD1 protein in breast cancer and normal tissues, Protein Medley (Takara Clontech) tissue lysates for human normal mammary gland, lung, heart, liver and kidney were used. Cultured breast cancer cells were harvested with lysis buffer containing 25 mM Tris-HCl (pH 7.4), 150 mM NaCl, 1 mM EDTA, 1% NP-40, 10% glycerol, 1% Phosphatase Inhibitor Cocktail Set II and 0.1% Protease Inhibitor Cocktail Set III (Calbiochem, San Diego, Calif.). SDS-PAGE and western blotting were performed as described previously (Park J H, Lin M L, Nishidate T, Nakamura Y and Katagiri T: PDZ-binding kinase/T-LAK cell-originated protein kinase, a putative cancer/testis antigen with an oncogenic activity in breast cancer. Cancer Res 66: 9186-9195, 2006). Antibodies used in this study were as follows; anti-RQCD1 rabbit polyclonal antibody (0.6 microgram/ml) for RQCD1, anti-beta-actin mouse monoclonal antibody (Ac-15) (SIGMA-Aldrich) (10 ng/ml) for ACTB, anti-Akt rabbit polyclonal antibody (#9272) (Cell Signaling Technology, Danvers, Mass., USA) (1:1,000 dilution) for Akt, anti-phospho Akt (Ser 473) mouse monoclonal antibody (#4051) (Cell Signaling Technology) (1:1,000 dilution) for phosphorylated Akt on Ser 473, anti-HA rat monoclonal antibody (3F10) (Roche, Basel, Switzerland) (20 ng/ml) for HA-tag, anti-Flag mouse monoclonal antibody (M2) (SIGMA-Aldrich) (25 ng/ml) for Flag-tag, and HRP-conjugated anti-mouse, rat or rabbit antibody (Santa Cruz Biotechnology, Santa Cruz, Calif., USA) (40 ng/ml).

8. Immunocytochemical Staining Analysis.

For immunocytochemical staining analysis, breast cancer cells, BT-549, were seeded at a density of 1×10⁵ cells per chamber of a 2-well Lab-Tek chamber slide (Nunc, Thermo Fisher Scientific, Waltham, Mass., USA). After 24-hour culture, cells were washed twice with phosphate buffered saline (PBS) (−), fixed with 4% paraformaldehyde solution at 4 degree C. for 15 min, and then permealized with PBS (−) containing 0.1% Triton X-100 at 4 degree C. for 3 min. Cells were covered with 5% bovine serum albumin (BSA) in PBS (−) at 4 degree C. for 60 min to block nonspecific binding before the primary antibody reaction. For the detection of endogenous RQCD1 in breast cancer cells, the cells were incubated with anti-RQCD1 rabbit polyclonal antibody (6 microgram/ml) with 5% BSA for 60 min and subsequently Alexa 488 anti-rabbit IgG (4 microgram/ml) (Molecular Probes, Eugene, Oreg., USA) with 5% BSA for 60 min. For the detection of exogenously expressed HA-tagged GIGYF1 and GIGYF2 proteins, cells were incubated with anti-HA rat monoclonal antibody (3F10) (0.4 microgram/ml) (Roche) with 5% BSA for 60 min, and subsequently Alexa 594 anti-rat IgG diluted 1:500 in 5% BSA for 60 min. Then, the cells were mounted with VEC-TASHIELD Mounting Medium with 4′, 6-diamino-2′-phenylindole dihydrochloride (DAPI) (Vector Laboratories, Burlingame, Calif., USA) to be counterstained their nuclei. Fluorescent images were obtained by TCS SP2 AOBS confocal microscope (Leica Microsystems, Wetzlar, Germany).

9. RNA Interference Assay.

The shRNA expression vectors were generated against RQCD1 by cloning of double-stranded oligonucleotides into the BbsI site in the psiU6BX3.0 vector as describe previously (Shimokawa T, Furukawa Y, Sakai M, Li M, Miwa N, Lin Y M and Nakamura Y: Involvement of the FGF18 gene in colorectal carcinogenesis, as a novel downstream target of the beta-catenin/T-cell factor complex. Cancer Res 63: 6116-6120, 2003). 1.0×10⁶ cells of BT-549 and HBC-4 cell lines were seeded in 10-cm plates. Twenty-four hours after seeding, the cells were transfected with each of shRNA expression vectors targeting RQCD1 (#1 and #2), or psiU6BX3.0 mock vector (without any insert) using FuGENE6 transfection reagent (Roche) according to the manufacturer's instructions. Twenty-four hours after transfection, the cells were reseeded to 6-Well Clear TC-Treated Microplates (Corning, Lowell, Mass., USA) (0.7×10⁵ cells/well) for cell proliferation and colony formation assays, and to 10-cm plates (3.5×10⁵ cells/plate) for RT-PCR and western blotting with culture medium containing 0.5 mg/ml of geneticine (Invitrogen). After geneticine treatment for 7 days, the knockdown effect of shRNA was examined by semi-quantitative RT-PCR and western blotting analyses as described above sections. After geneticine treatment for 8 days, cell proliferation assays were performed with Cell Counting Kit-8 (Dojindo, Kumamoto, Japan). Moreover, after geneticine treatment for 9 days, colony formation assays were performed by staining colonies with giemsa staining solution (Merck, Whitehouse Station, N.J., USA) following fixation by 4% paraformaldehyde for 15 min. For Akt activation analysis, each of the oligo-duplex siRNAs (SIGMA-Genosys, St Louis, Mo., USA) targeting RQCD1 (#1), GIGYF1 or GIGYF2, and also siRNA for EGFP were used as a control. Seventy-two hours after transfection of siRNAs, Akt activity was evaluated by western blotting with anti-phospho Akt mouse monoclonal antibody which can recognize the phosphorylation of Akt at Ser 473 (#4051; Cell Signaling Technology). To evaluate the effects on the level of Akt phosphorylation after knockdown of RQCD1, GIGYF1 or GIGYF2, the band intensities of western blotting with anti-phospho-Akt and anti-Akt-antibodies were quantified by using Image J analysis software (http://rsb.info.nih.gov/ij) (Abramoff M D, Magelhaes P J and Ram S J, Image Processing with Image J. Biophotonics International 11: 36-42, 2004). The siRNA target sequences were as follows;

(SEQ ID NO: 30)
5′-GATCTATCAGTGGATCAAT-3′
for RQCD1 (#1),
(SEQ ID NO: 31)
5′-GATCTTGTTAGATGACACT-3′
for RQCD1 (#2),
(SEQ ID NO: 32)
5′-CCTTCCGAAGGGCTAGAGG-3′
for GIGYF1,
(SEQ ID NO: 33)
5′-CAAGATACCTTCAGACCTT-3′
for GIGYF2,
and
(SEQ ID NO: 34)
5′-GCAGCACGACTTCTTCAAG-3′
for EGFP.

10. Preparation of RQCD1 Stably-Expressing Cell Lines.

HEK293 cells were transfected with pCAGGS-HA-RQCD1 plasmid vector or mock plasmid vector using FuGENE6 transfection reagent (Roche). Twenty-four hours after transfection, cells were incubated in culture medium with 0.5 mg/ml of Geneticine (Invitrogen) for 14 days. Then, more than 20 individual colonies were isolated, and then each colony was evaluated for its monoclonal expression of RQCD1 protein by immunocytochemical staining and western blotting with anti-HA antibody. Finally, three independent clones were established and designated as follows: HEK293-RQCD1-1, -2, and -3 (stable-1, -2 and -3), and HEK293-Mock-1, -2, and -3 (mock-1, -2 and -3). For cell proliferation assay, mock- and RQCD1-stable cell lines were seeded to collagen Type1 coated 6-well microplate (Asahi glass co, Tokyo, Japan) (0.4×10⁵ cells/well), and cell growth was evaluated with Cell Counting Kit-8 (Dojindo) according to the manufacturer's instructions.

11. GST-Pull Down Assay.

HBC-4 was seeded at 1.0×10⁶ cells in 10-cm plate. Twenty-four hours after seeding, the cells were harvested with 500 microlitter of ice-cold buffer containing 25 mM Tris-HCl (pH 7.4), 150 mM NaCl, 1 mM EDTA, 0.1% NP-40, 10% glycerol, 1% Phosphatase Inhibitor Cocktail Set II and 0.1% Protease Inhibitor Cocktail Set III (Calbiochem), cleared by centrifugation at 18,000×g for 15 min and rotated with 30 microlitter of Sepharose-4B (SIGMA-Aldrich) for 1 h at 4 degree C. for pre-clear. Then, 1 microgram of GST alone or the same molecular number of the N-terminally GST-fused full-length RQCD1 recombinant protein was added to supernatants, and rotated for 1 h at 4 degree C. Then, 10 microlitter of Glutathione Sepharose-4B (Zymed Laboratories, South San Francisco, Calif., USA) was added to each cell lysate and rotated for 1 h at 4 degree C. Sepharose beads were washed with 500 microlitter of lysis buffer for three times, and then bound proteins were eluted by addition of 30 microlitter of SDS-PAGE sample buffer.

12. Mass Spectrometric Analysis.

Eluted samples of GST-pull down assay were loaded onto SDS-PAGE followed by silver staining with SilverQuest Silver Staining Kit (invitrogen). The excised protein bands were reduced in 10 mM Tris (2-carboxyethyl) phosphine (Sigma-Aldrich) with 50 mM ammonium bicarbonate (Sigma-Aldrich) for 30 min at 37 degree C. and alkylated in 50 mM iodoacetamide (Sigma-Aldrich) with 50 mM ammonium bicarbonate for 45 min in the dark at 25 degree C. Porcine trypsin (Promega, San Luis Obispo, Calif.) was added for a final enzyme: protein ratio of 1:20 and incubated at 37 degree C. for 16 h. The resulting peptide mixture was separated on a 100 micrometer×150 millimeter HiQ-Sil (KYA Technologies, Tokyo, Japan) using 30 min linear gradient from 5.4 to 29.2% acetonitrile in 0.1% trifluoroacetic acid (TFA) with total flow of 300 nl/min. The eluting peptides were mixed with matrix solution (4 mg/ml alpha-cyano-4-hydroxy-cinnamic acid, 0.08 mg/ml of ammonium citrate in 70% acetonitrile, 0.1% TFA) and automatically spotted onto MALDI target plates by MaP (KYA Technologies, Tokyo, Japan). Mass spectrometric analysis was performed on 4800 Plus MALDI/TOF/TOF Analyzer (Applied Biosystems/MDS Sciex). MS/MS peak list generated by the Protein Pilot version 2.0.1 software (Applied Biosystems/MDS Sciex) was exported to a local MASCOT version 2.2.03 search engine (Matrix Science, Boston, Mass., USA) for protein data base search.

13. Co-Immunoprecipitation Assay.

HEK293T cells were seeded at the density of 1.0×10⁶ cells/10-cm plate. Twenty-four hours after seeding, the cells were transfected with indicated combinations of expression vectors. At thirty-six hours after transfection, cells were harvested with 500 microlitter of ice-cold buffer containing 25 mM Tris-HCl (pH 7.4), 150 mM NaCl, 1 mM EDTA, 0.1% NP-40, 10% glycerol, 1% Phosphatase Inhibitor Cocktail Set II and 0.1% Protease Inhibitor Cocktail Set III (Calbiochem), cleared by centrifugation at 18,000×g at 4 degree C. for 15 min, and rotated with 10 microlitter of anti-Flag (M2) agarose (SIGMA-Aldrich) or anti-HA (HA-7) agarose (SIGMA-Aldrich) at 4 degree C. for 1 h. Then, those agarose beads were washed with 500 microlitter of washing buffer containing 25 mM Tris-HCl (pH 7.4), 150 mM NaCl, 1 mM EDTA, 0.1% NP-40, 10% glycerol for three times, and 500 microlitter of buffer containing 25 mM Tris-HCl (pH 7.4), 150 mM NaCl, 1 mM EDTA. Finally, precipitated proteins were eluted by incubating with 20 microlitter of 150 microgram/ml 3xFlag peptide (SIGMA-Aldrich) or 200 microgram/ml HA peptide (SIGMA-Aldrich) at 4 degree C. for 1 h, respectively.

14. Statistical Analysis.

Statistical significance was examined by Student's t-test. A difference of P<0.05 was considered to be statistically significant.

### II. Results

1. RQCD1 is Up-Regulated in Breast Cancer Cells.

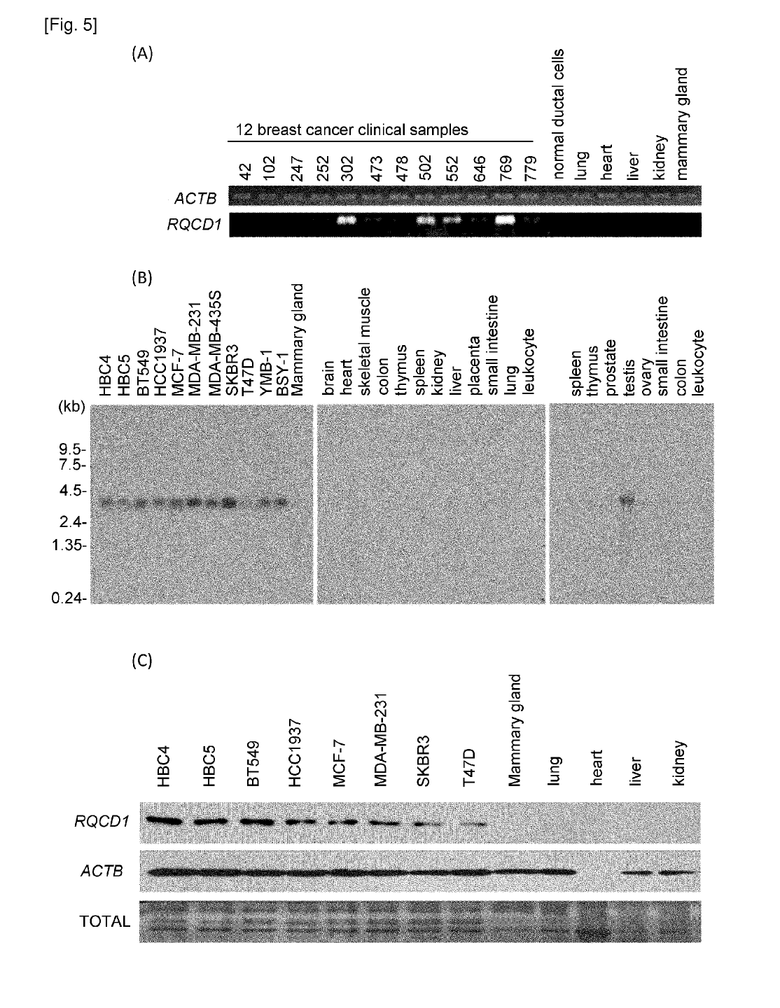

To screen molecules that could be applicable as targets for development of novel therapeutic drugs, genome-wide expression profile analysis of 81 breast cancer specimens were carried out by cDNA microarray targeting 23,040 cDNAs or ESTs (Nishidate T, Katagiri T, Lin M L et al: Genome-wide gene-expression profiles of breast-cancer cells purified with laser microbeam microdissection: identification of genes associated with progression and metastasis. Int J Oncol 25: 797-819, 2004). Among dozens of up-regulated genes, the present invention focused on RQCD1, whose expression was frequently up-regulated (at least 3-fold more than normal ductal cells) in a solid-tubular type of breast carcinoma. Its overexpression was confirmed in 4 of 12 clinical solid-tubular cases by comparison with normal breast ductal cells or with whole mammary gland by semiquantitative RT-PCR (FIG. 5A). Subsequent northern blotting analysis using a RQCD1 cDNA fragment revealed overexpression of its transcript (approximately 3.5 kb long) in breast cancer cell lines, while RQCD1 expression was very weak or hardly detectable in normal human organs except the testis (FIG. 5B) as concordant to the results of cDNA microarray analysis. Since the assembled cDNA sequence of RQCD1 (Accession No. NM_{—}005444; 900 bp) in the NCBI database was smaller than the size of the transcript indicated by northern-blot analysis, the exon-connection, and 5′ and 3′ RACE experiments were performed. The full-length cDNA sequences of human RQCD1 were obtained, consisting of 3,284 nucleotides (Genbank accession; AB500892) encoding a protein of 299 amino acids. The RQCD1 gene consists of eight exons and spans an approximately 45.4-kb genomic region on chromosomal band 2q35.

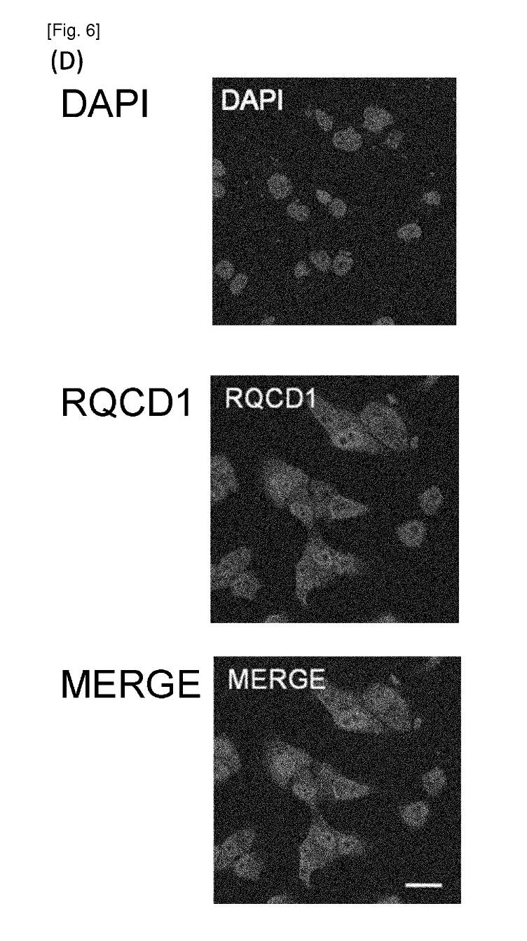

To investigate the expression of RQCD1 protein in breast cancer cells, a polyclonal antibody was generated against full-length RQCD1 protein, and performed western blotting analysis using the whole cell lysate from eight breast cancer cell lines as well as normal human tissues including mammary gland, lung, heart, liver and kidney. A high level of RQCD1 protein was detected in all the breast cancer cell lines examined, though its expression was hardly detectable in any of normal human tissues except the testis (FIG. 5C). Furthermore, the subcellular localization of endogenous RQCD1 protein in a breast cancer cell line, BT-549 was examined by immunocytochemical staining analysis using the purified anti-RQCD1 polyclonal antibody. It was observed diffusely in both cytoplasm and nucleus of breast cancer cells (FIG. 6D).

2. Effect of ROCD 1 on Cell Growth.

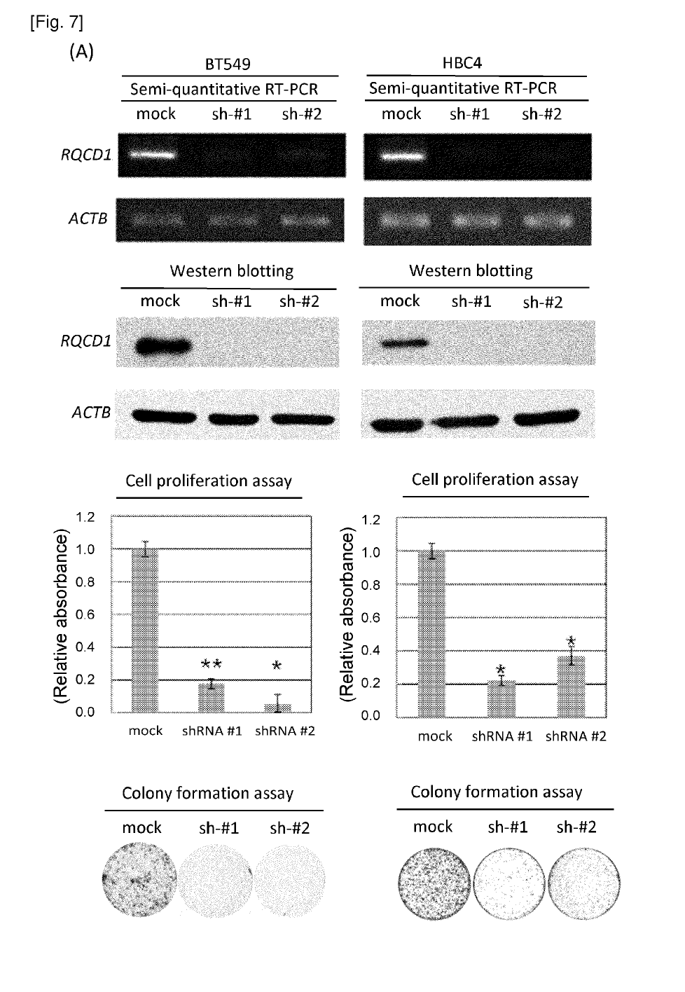

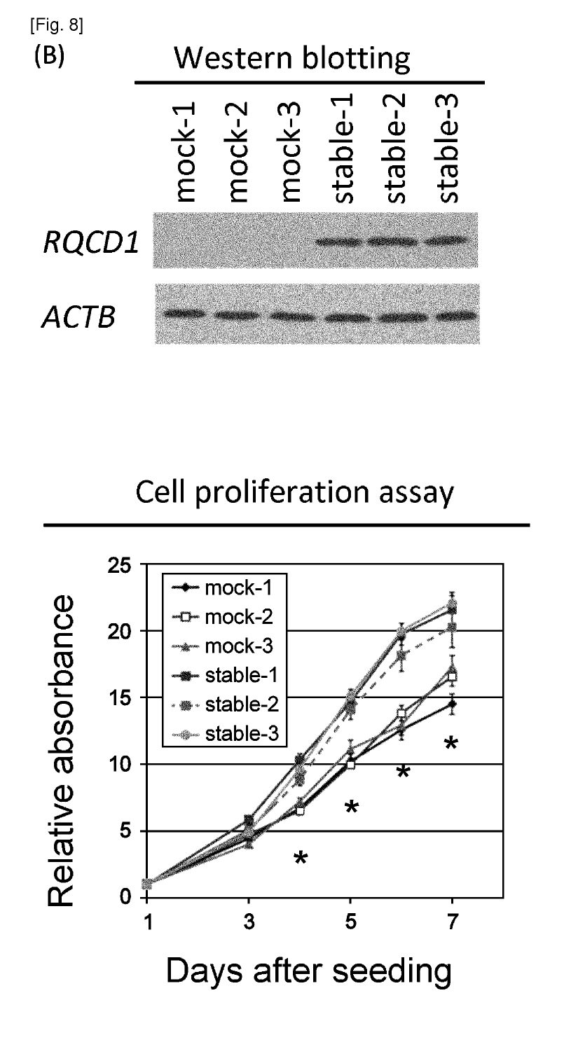

To examine the functional role of RQCD1 in breast cancer cell growth, the expression of endogenous RQCD1 was knocked down in breast cancer cell lines, BT-549 and HBC-4, which showed high RQCD1 expression at both transcriptional and protein levels (FIG. 7), by means of small hairpin-RNA (shRNA) expression vector system. Semiquantitative RT-PCR and western blotting analyses indicated that RQCD1-specific shRNAs (shRNA#1 (sh-#1) and shRNA#2 (sh-#2)) significantly suppressed RQCD1 expression while no change was observed in the MOCK-transfected cells (FIG. 7A). Then cell-proliferation and colony formation assays were performed, and it was discovered that introduction of shRNA#1 and shRNA#2 constructs significantly suppressed growth of both BT-549 and HBC-4-cells (BT-549: shRNA#1, P=0.004 and shRNA#2, P=0.002; HBC-4: shRNA#1, P=0.002 and shRNA#2, P=0.002; Student's t-test), in concordance with the results of knockdown effect of the transcript (FIG. 7A). To further confirm the growth-promoting effect of RQCD1, three independent HEK293 derivative cells were established that stably expressed exogenous RQCD1 at high level (stable-1, -2 and -3) compared to parental HEK293 (FIG. 8B). Subsequent cell proliferation assay revealed that the three RQCD1-stable cells (stable-1, -2 and -3) grew significantly much faster than those transfected with mock plasmid (mock-1, -2 and -3; FIG. 8B right panel), indicating an oncogenic role of RQCD1 overexpression.

3. Identification of Molecules Interacting with RQCD1.

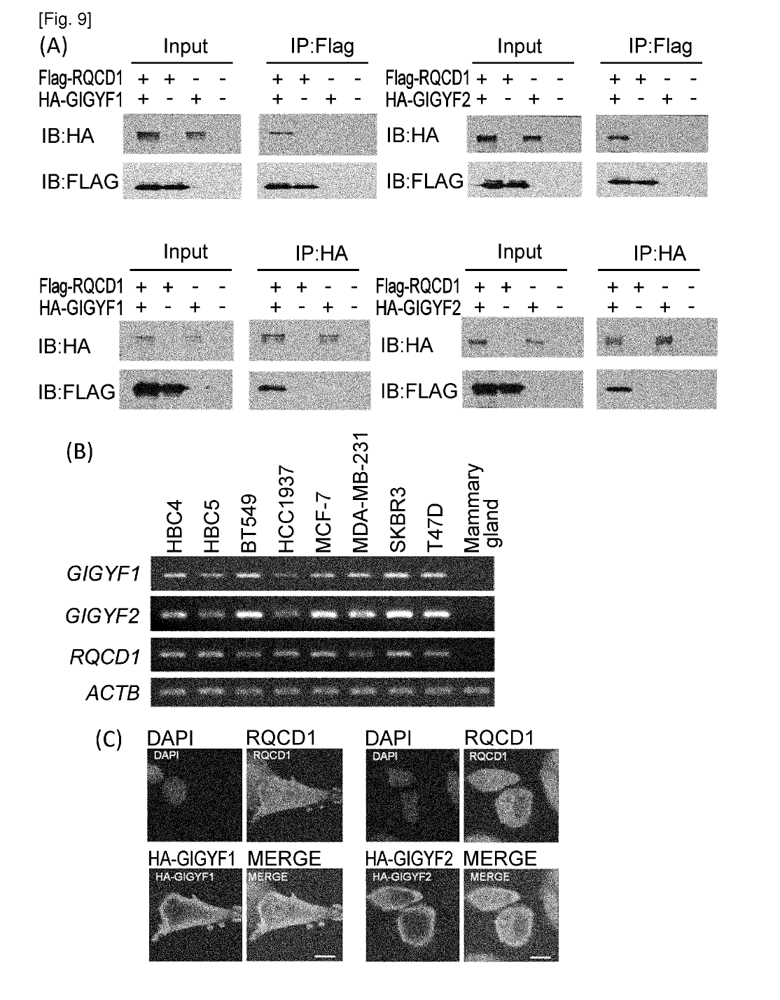

To further investigate its biological function, a protein(s) interacting with RQCD1 protein in breast cancer cells was saught by GST-pull down assay using the N-terminally GST-fused full-length RQCD1 recombinant protein (GST-RQCD1) and mass spectrometric analysis (see Materials and methods, Example 2). Comparison of silver staining patterns of SDS-PAGE gels containing the pulled-down proteins identified two proteins, approximately at 140 kDa and 160 kDa specifically in the lane corresponding to proteins pulled-down with GST-RQCD1 protein (data not shown). Mass spectrometric analysis indicated these 140 kDa and 160 kDa proteins to be Grb10-interacting GYF protein 1 (GIGYF1) and 2 (GIGYF2), respectively, which were previously indicated their involvement in the PI3K/Akt signaling pathway (Giovannone B, Lee E, Laviola L, Giorgino F, Cleveland K A and Smith R J: Two novel proteins that are linked to insulin-like growth factor (IGF-1) receptors by the Grb10 adapter and modulate IGF-1 signaling. J Biol Chem 34: 31564-31573, 2003). Subsequently, to confirm the interaction between RQCD1 and GIGYF1/GIGYF2, co-immunoprecipitation assays were performed (see Materials and Methods, Example 2). Flag-tagged RQCD1 (Flag-RQCD1), and HA-tagged GIGYF1 or GIGYF2 (HA-GIGYF1, HA-GIGYF2) constructs were co-transfected into HEK-293T cells, and the cell lysates were immunoprecipitated with anti-Flag antibody. Immunoblotting of the precipitates with anti-HA antibodies suggested co-immunoprecipitation of Flag-RQCD1 with HA-GIGYF1 or HA-GIGYF2. Conversely, immunoprecipitation was also carried out with anti-HA antibody and subsequent immunoblotting of precipitates with anti-Flag antibody, and confirmed their co-immunoprecipitation (FIG. 9A). Then, the transcriptional levels of GIGYF1 and GIGYF2 were examined in breast cancer cell lines by semi-quantitative RT-PCR, and found that GIGYF1 and GIGYF2 were also up-regulated in all breast cancer cell lines examined, compared with normal mammary gland (FIG. 9B). The subcellular localization of these proteins in breast cancer cells, BT-549, was further examined by immunocytochemical staining, and detected HA-GIGYF1 and HA-GIGYF2 proteins in cytoplasm, and partially colocalized with endogenous RQCD1.

4. Involvement of RQCD1 in Akt-Signaling Pathway.

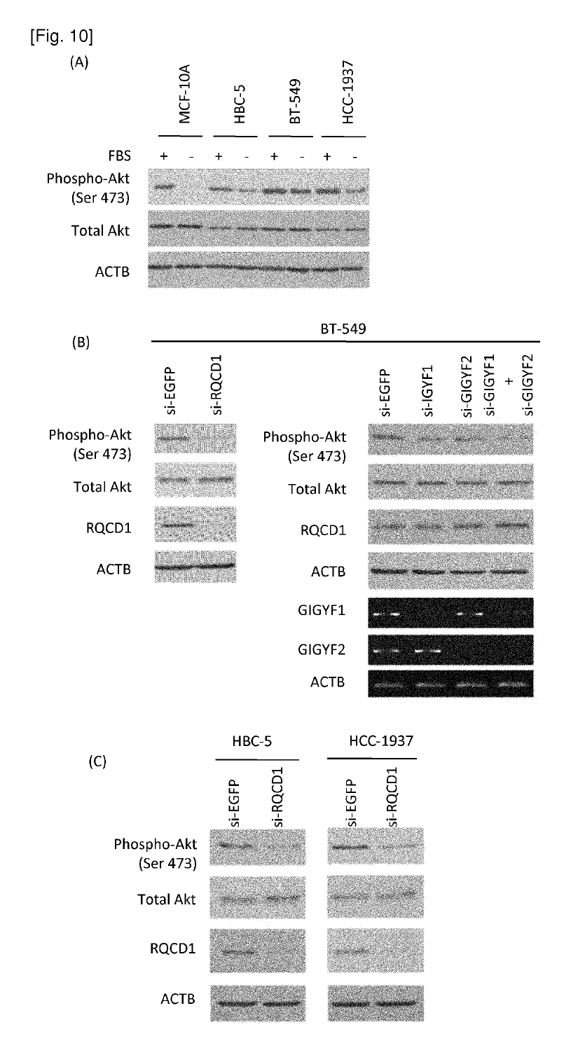

Since overexpression of GIGYF1 and GIGYF2 was reported to activate PI3K/Akt signaling pathway in mouse embryonic fibroblasts that were transfected with the IGF-I receptor (Giovannone B, Lee E, Laviola L, Giorgino F, Cleveland K A and Smith R J: Two novel proteins that are linked to insulin-like growth factor (IGF-1) receptors by the Grb10 adapter and modulate IGF-1 signaling. J Biol Chem 34: 31564-31573, 2003), herein it was examined whether RQCD1, GIGYF1 and GIGYF2 could effect on the Akt activity. The phosphorylation of Akt at Ser 473 in its carboxyl-terminal hydrophobic motif is known to be a representative marker for activation of Akt (Alessi DR, Andjelkovic M, Caudwell B, Cron P, Morrice N, Cohen P and Hemmings BA: Mechanism of activation of protein kinase B by insulin and IGF-1. EMBO J. 15: 6541-6551, 1996, Yang J, Cron P, Thompson V, Good V M, Hess D, Hemmings B A and Barford D: Molecular mechanism for the regulation of protein kinase B/Akt by hydrophobic motif phosphorylation. Mol Cell 9: 1227-1240, 2002, Scheid M P, Marignani P A and Woodgett J R: Multiple phosphoinositide 3-kinase-dependent steps in activation of protein kinase B. Mol Cell Biol 22: 6247-6260, 2002). Therefore, western blotting was first performed with anti-Akt and anti-phospho-Akt (Ser 473) antibodies to examine the Akt activity status in breast cancer cells, BT-549, HBC-5 and HCC-1937, which showed a high level of RQCD1 expression (FIG. 9C). The results showed that the high level of phosphorylation of Akt at Ser 473 was clearly observed in all breast cancer cells even in the absence of the serum stimulation, while its phosphorylation was abolished in normal ductal epithelial cell-derived MCF-10A in the serum-depletion condition (FIG. 10A), indicating that Akt is constitutively activated in these breast cancer cells. The knockdown effects of RQCD1, GIGYF1 or GIGYF2 expressions were then investigated by siRNA treatments on the Akt phosphorylation level, and found that treatment of each siRNA against either RQCD1, GIGYF1 or GIGYF2 into BT-549 cells caused the significant reduction of phosphorylation level of Akt without alteration of total Akt protein level (FIG. 10B, 11D). A similar effect on the Akt activity was also observed by the RQCD1-siRNA treatment in the other breast cancer cell lines, HBC-5 and HCC-1937 (FIG. 10C, 11D).

### III. Discussion

Molecular targeting drugs for breast cancer therapy have contributed to reduction in motility rate and improvement in QOL of patients in the last two decades (Parkin D M, Bray F, Ferlay J: Global cancer statistics, 2002. CA Cancer J Clin 55: 74-108, 2005, Veronesi U, Boyle P, Goldhirsch A, Orecchia R, Viale G: Breast cancer. Lancet 365: 1727-1741, 2005). However, the proportion of patients showing good response to presently available treatments is still limited particularly for the patients at advanced stages or those with triple-negative breast cancer (Johannes B, Esther Z and Axel U, Nat Med 7: 548-552, 2001). Toward identification of molecular targets for drug development, the detailed gene expression profiles of 81 clinical breast cancer cells (Nishidate T, Katagiri T, Lin M L et al: Genome-wide gene-expression profiles of breast-cancer cells purified with laser microbeam microdissection: identification of genes associated with progression and metastasis. Int J Oncol 25: 797-819, 2004) and 29 normal human tissues (Saito-Hisaminato A, Katagiri T, Kakiuchi S, Nakamura T, Tsunoda T and Nakamura Y., DNA Res 9: 35-45, 2002) were analyzed for selecting genes that were up-regulated specifically in breast cancer cells in combination with experiments screening for knock down effects by means of the RNA interference system. On the basis of this approach, RQCD1 was found to be up-regulated frequently in clinical breast cancer samples as well as breast cancer cell lines, while its expression was very low in normal human tissues except the testis. These results indicated RQCD1 as a novel cancer-testis antigen. Furthermore, knockdown of RQCD1 expression was demonstrated to result in significant growth suppression of breast cancer cells and that introduction of RQCD1 into HEK293 cells significantly promoted the cell growth, implying that RQCD1 could serve as a valuable target for development of anticancer agents or cancer peptide vaccine for breast cancer.

RQCD1, a protein evolutionarily conserved among eukaryotes, was first identified as a crucial factor for regulation of differentiation in nitrogen-starved fission yeast; yeast cells lacking of RQCD1 were reported to be sterile when they were cultured in the nitrogen-starvation condition (Okazaki N, Okazaki K, Watanabe Y, Kato-Hayashi M, Yamamoto M and Okayama H, Mol Cell Biol 18: 887-895, 1998). Furthermore, the murine homolog of RQCD1 was reported as a transcriptional cofactor that mediated retinoic acid-induced differentiation and also to be an erythropoietin-responsive gene potentially involved in development of hematopoietic cell (Hiroi N, Ito T, Yamamoto H, Ochiya T, Jinno S, Okayama H., EMBO J. 21: 5235-5244, 2002, Gregory R C, Lord K A, Panek L B, Gaines P, Dillon S B and Wojchowski D M: Subtraction cloning and initial characterization of novel Epo-immediate response genes. Cytokine 12: 845-857, 2000). However, since its biological roles in tumorigenesis have not been investigated, interacting proteins of RQCD1 were saughtr and the interaction of RQCD1 with both GIGYF1 and GIGYF2 proteins that have been reported to be linked to IGF-1 receptors was identified (Giovannone B, Lee E, Laviola L, Giorgino F, Cleveland K A and Smith R J, J Biol Chem 34: 31564-31573, 2003). The knockdown of RQCD1, GIGYF1 or GIGYF2 was further confirmed by siRNA treatment resulted in reduction of the phosphorylation level of Akt at Ser 473, that is known to be a marker of its activation (Alessi D R, Andjelkovic M, Caudwell B, Cron P, Morrice N, Cohen P and Hemmings B A, EMBO J. 15: 6541-6551, 1996; Yang J, Cron P, Thompson V, Good V M, Hess D, Hemmings B A and Barford D, Mol Cell 9: 1227-1240, 2002; Scheid M P, Marignani P A and Woodgett J R: Multiple phosphoinositide 3-kinase-dependent steps in αtivation of protein kinase B. Mol Cell Biol 22: 6247-6260, 2002), in breast cancer cells in which these genes was overexpressed.

### INDUSTRIAL APPLICABILITY

The gene-expression analysis of cancers described herein using the combination of laser-capture dissection and genome-wide cDNA microarray has identified specific genes as targets for cancer prevention and therapy. Based on the expression of a differentially expressed gene, RQCD1, GIGYF1 and GIGYF2, the present invention provides molecular diagnostic markers for identifying and detecting cancer, in particular, breast cancer.

The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provide indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for diagnosing cancer or a predisposition for developing cancer in a subject, comprising the step of determining the expression level of an RQCD1 gene, a GIGYF1 gene or a GIGYF2 gene in a subject-derived biological sample, wherein an increase in said expression level as compared to a normal control level of said gene indicates that said subject suffers from or is at a risk of developing cancer, wherein said expression level is determined by any of method selected from the group consisting of:
(a) detecting mRNA of an RQCD1 gene, a GIGYF 1 gene or a GIGYF2 gene;
(b) detecting a protein encoded by an RQCD1 gene, a GIGYF 1 gene or a GIGYF2 gene; and
(c) detecting a biological activity of a protein encoded by an RQCD1 gene, a GIGYF1 gene or a GIGYF2 gene.

2. The method of claim 1, wherein said expression level is at least 10% greater than the normal control level.

3. The method of claim 1, wherein said subject-derived biological sample comprises a biopsy.

4. The method of claim 1, wherein the cancer is breast cancer.

5. A kit for detecting cancer comprising a detection reagent which binds to a transcription or translation product of an RQCD1 gene, a GIGYF1 gene or a GIGYF2 gene.

6. A method of screening a candidate agent for treating or preventing cancer, which comprises steps of:
(a) contacting a test agent with an RQCD1 polypeptide, a GIGYF1 polypeptide or a GIGYF2 polypeptide or a fragment thereof;
(b) detecting binding between the polypeptide or fragment and the test agent; and
(c) selecting the test agent that binds to the polypeptide or fragment as a candidate agent for treating or preventing cancer.

7. A method of screening a candidate agent for treating or preventing cancer, wherein said method comprises steps of:
(a) contacting a test agent with an RQCD1 polypeptide, a GIGYF1 polypeptide or a GIGYF2 polypeptide or a fragment thereof;
(b) detecting a biological activity of the polypeptide or fragment;
(c) comparing the biological activity of the polypeptide or fragment with the biological activity detected in the absence of the agent; and
(d) selecting the agent that suppresses the biological activity of the polypeptide as a candidate agent for treating or preventing cancer.

8. The method of claim 7, wherein the biological activity is cell proliferative activity or Akt phosphorylation activity.

9. A method of screening a candidate agent for treating or preventing cancer, which comprises steps of:
(a) contacting a test agent with a cell expressing an RQCD1 gene, a GIGYF 1 gene or a GIGYF2 gene or a cell introduced with a vector that comprises a transcriptional regulatory region of an RQCD1 gene, a GIGYF 1 gene or a GIGYF2 gene and a reporter gene expressed under control of the transcriptional regulatory region;
(b) detecting expression level of the RQCD1 gene, the GIGYF 1 gene or the GIGYF2 gene or measuring expression level or activity of said reporter gene;
(c) comparing the expression level with the expression level or activity detected in the absence of the agent; and
(d) selecting the agent that reduces the expression level or activity as a candidate agent for treating or preventing breast cancer.

10. (canceled)

11. A double-stranded molecule, when introduced into a cell expressing an RQCD1 gene, the GIGYF 1 gene or the GIGYF2 gene, inhibits expression of the gene, which molecule comprises a sense strand and an antisense strand, wherein the sense strand comprises a nucleotide sequence selected from the group consisting of SEQ ID NOs: 8, 9, 30, 31, 32 and 33 as a target sequence, and the antisense strand comprises a nucleotide sequence complementary to the target sequence of the sense strand so that the sense and antisense strands hybridize to each other to form the double-stranded molecule.

12. The double-stranded molecule of claim 11, wherein the sense strand comprises from about 19 to about 25 contiguous nucleotides from the nucleotide sequences selected from the group consisting of SEQ ID NOs: 10, 35 and 37.

13. The double-stranded molecule of claim 11, wherein said double-stranded molecule is a single nucleotide construct comprising the sense strand and the antisense strand linked via a single-strand.

14. The double-stranded molecule of claim 13, which has a general formula 5′-[A]-[B]-[A′]-3′, wherein [A] is a sense strand, [B] is a consists single strand consisting of 3 to 23 nucleotides, and [A′] is an antisense strand.

15. The vector encoding the double-stranded molecule of claim 11.

16. The vector of claim 15, wherein the vector encodes a transcript which has a secondary structure and comprises the sense strand and the antisense strand.

17. The vector of claim 16, wherein the transcript further comprises a single strand linking said sense strand and said antisense strand.

18. The vector of claim 17, wherein the transcript has the general formula 5′-[A]-[B]-[A′]-3′, wherein [A] is the sense strand; [B] is a single strand consisting of 3 to 23 nucleotides; and [A′] is the antisense strand.

19. Vectors comprising each of a combination of polynucleotide comprising a sense strand nucleic acid and an antisense strand nucleic acid, wherein said sense strand nucleic acid comprises the nucleotide sequence of SEQ ID NOs: 8, 9, 30, 31, or 32 and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the RQCD1, GIGYF1 or GIGYF2 gene, inhibits the cell proliferation.

20. A method of treating or preventing cancer in a subject comprising administering to said subject a pharmaceutically effective amount of a double-stranded molecule against a RQCD1 gene, a GIGYF 1 gene or a GIGYF2 gene, or a vector comprising said double-stranded molecule, which double-stranded molecule inhibits the expression of the RQCD1 gene, the GIGYF1 gene or the GIGYF2 gene, and a pharmaceutically acceptable carrier.

21. The method of claim 20, wherein a double-stranded molecule, when introduced into a cell expressing an RQCD1 gene, the GIGYF 1 gene or the GIGYF2 gene, inhibits expression of the gene, which molecule comprises a sense strand and an antisense strand, wherein the sense strand comprises a nucleotide sequence selected from the group consisting of SEQ ID NOs: 8, 9, 30, 31, 32 and 33 as a target sequence, and the antisense strand comprises a nucleotide sequence complementary to the target sequence of the sense strand so that the sense and antisense strands hybridize to each other to form the double-stranded molecule, wherein the vector encodes the double-stranded molecule.

22. The method of claim 20, wherein the cancer is selected from the group consisting of breast cancer, lung cancer and esophagus cancer.

23. A composition for treating or preventing cancer, which comprises a pharmaceutically effective amount of a double-stranded molecule against a RQCD1, gene, a GIGYF 1 gene or a GIGYF2 gene or a vector comprising said double-stranded molecule, which double-stranded molecule inhibits the expression of the RQCD1 gene, the GIGYF1 gene or the GIGYF2 gene, and a pharmaceutically acceptable carrier.

24. The composition of claim 23, wherein the double-stranded molecule, when introduced into a cell expressing an RQCD1 gene, the GIGYF 1 gene or the GIGYF2 gene, inhibits expression of the gene, which molecule comprises a sense strand and an antisense strand, wherein the sense strand comprises a nucleotide sequence selected from the group consisting of SEQ ID NOs: 8, 9, 30, 31, 32 and 33 as a target sequence, and the antisense strand comprises a nucleotide sequence complementary to the target sequence of the sense strand so that the sense and antisense strands hybridize to each other to form the double-stranded molecule, wherein the vector encodes the double-stranded molecule

25. The composition of claim 23, wherein the cancer is selected from the group of breast cancer, lung cancer and esophagus cancer.

26. A method of screening for a candidate agent for treating or preventing cancer, said method including steps of:
(a) contacting a GIGYF 1 polypeptide and/or a GIGYF2 polypeptide or functional equivalent thereof with a RQCD1 polypeptide or functional equivalent thereof in the presence of a test agent;
(b) detecting the binding between the polypeptides of step (a); and
(c) selecting the test agent that inhibits the binding between the GIGYF 1 polypeptide or the GIGYF2 polypeptide and the RQCD1 polypeptides.

27. A kit for screening for a candidate agent for treating or preventing cancer, said kit including:
(a) a GIGYF 1 polypeptide and/or a GIGYF2 polypeptide or functional equivalent thereof, and
(b) a RQCD1 polypeptide or functional equivalent thereof.
